(19) United States
(12) Patent Application Publication
(10) Pub. No.: US 2008/0050726 A1
(43) Pub. Date: Feb. 28, 2008

(54) Methods for diagnosing pancreatic cancer

(76) Inventors: Yixin Wang, San Diego, CA (US); Dmitri Talantov, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/523,496

(22) Filed: Sep. 19, 2006

Related U.S. Application Data
(60) Provisional application No. 60/718,501, filed on Sep. 19, 2005.
(60) Provisional application No. 60/725,680, filed on Oct. 12, 2005.

(51) Int. Cl. C12Q 1/68; C07H 21/04; C40B 40/08

(52) U.S. Cl. 435/6; 506/17; 536/23.5

(57) ABSTRACT

The present invention provides a method of identifying origin of a metastasis of unknown origin by obtaining a sample containing metastatic cells; measuring Biomarkers associated with at least two different carcinomas; combining the data from the Biomarkers into an algorithm where the algorithm normalizes the Biomarkers against a reference; and imposes a cut-off which optimizes sensitivity and specificity of each Biomarker, weights the prevalence of the carcinomas and selects a tissue of origin determining origin based on highest probability determined by the algorithm or determining that the carcinoma is not derived from a particular set of carcinomas; and optionally measuring Biomarkers specific for one or more additional different carcinoma, and repeating the steps for additional Biomarkers.

### PARENT CASE TEXT

This application claims the benefit of U.S. provisional patent application Ser. Nos. 60/718,501 filed Sep. 19, 2005; and 60/725,680 filed Oct. 12, 2005.

### STATEMENT REGARDING FEDERALLY SPONSORED RESEARCH OR DEVELOPMENT

No government funds were used to make this invention.

### REFERENCE TO SEQUENCE LISTING, OR A COMPUTER PROGRAM LISTING COMPACT DISK APPENDIX

Reference to a “Sequence Listing”, a table, or a computer program listing appendix submitted on a compact disc and an incorporation by reference of the material on the compact disc including duplicates and the files on each compact disc shall be specified.

### BACKGROUND OF THE INVENTION

Pancreatic cancer is a deadly disease which has a mortality rate in the United States of more than 27,000 people a year. Lillemoe et al (2000). About 85% of those diagnosed with the disease have metastasis or spread of the disease beyond the pancreas and are almost impossible to cure with surgical resection. If the growth is found sooner it may be resected with a much better hope of cure. Only 20% of the tumors are resectable and the survival benefit of approved chemotherapy regiments is rather poor and the chances of a cure are usually 25% or less. Kroep et al. (1999); Wiesenauer et al. (2003); Ros et al. (2001); Ryu et al. (2002); and Ito et al. (2001). Earlier diagnosis is necessary for earlier successful treatment.

Despite the advances in diagnostic imaging methods like ultrasonography (US), endoscopic ultrasonography (EUS), dualphase spiral computer tomography (CT), magnetic resonance imaging (MRT), endoscopic retrograde cholangiopancreatography (ERCP) and transcutaneous or EUS-guided fine-needle aspiration (FNA), distinguishing pancreatic carcinoma from benign pancreatic diseases, especially chronic pancreatitis, is difficult because of the similarities in radiological and imaging features and the lack of specific clinical symptoms for pancreatic carcinoma.

Substantial efforts have been directed to developing tools useful for early diagnosis of pancreatic carcinomas. Nonetheless, a definitive diagnosis is often dependent on exploratory surgery which is inevitably performed after the disease has advanced past the point when early treatment may be effected. 20060029987.

Neoplasms of the exocrine pancreas may arise from ductal, acinar and stromal cells. Eighty percent of pancreatic carcinomas are derived from ductal epithelium. 60% of these tumors are located in the head of the pancreas, 10% in the tail and 30% are located in the body of the pancreas or are diffuse. Warshau et al. (1992). Histologically, these tumors are graded as well as differentiated, moderately differentiated and poorly differentiated. Some tumors are classified as adenosquamous, mucinous, undifferentiated or undifferentiated with osteoblast-like giant cells. Gibson et al. (1978).

Various gene expression profiles and genetic markers related to pancreatic cancer have been put forth. 20050009067; 20040219572; and 20030212264.

### BRIEF DESCRIPTION OF THE DRAWINGS

FIG. 1 depicts microarray data showing intensities of two genes in a panel of tissues. (A) Prostate stem cell antigen (PSCA). (B) Coagulation factor V (F5). The bar graphs show the intensity on the y-axis and the tissue on the x-axis. Panc Ca, pancreatic cancer; Panc N, normal pancreas.

FIG. 2 depicts electropherograms obtained from an Agilent Bioanalyzer. RNA was isolated from FFPE tissue using a three hour (A) or sixteen hour (B) proteinase K digestion. Sample C22 (red) was a one-year old block while sample C23 (blue) was a five-year old block. A size ladder is shown in green.

FIG. 3 depicts a comparison of Ct values obtained from three different qRTPCR methods: random hexamer priming in the reverse transcription followed by qPCR with the resulting cDNA (RH 2 step), gene-specific (reverse primer) priming in the reverse transcription followed by qPCR with the resulting cDNA (GSP 2 step), or gene-specific priming and qRTPCR in a one-step reaction (GSP 1 step). RNA from eleven samples was divided into the three methods and RNA levels for three genes were measured: β-actin (A), HUMSPB (B), and TTF (C). The median Ct value obtained with each method is indicated by the solid line.

FIG. 4 depicts assay optimization. (A and B) Electropherograms obtained from an Agilent Bioanalyzer. RNA was isolated from FFPE tissue using a three hour (A) or sixteen hour (B) proteinase K digestion. Sample C22 (red) was a one-year old block while sample C23 (blue) was a five-year old block. A size ladder is shown in green. (C and D) Comparison of Ct values obtained from three different qRTPCR methods: random hexamer priming in the reverse transcription followed by qPCR with the resulting cDNA (RH 2 step), gene-specific (reverse primer) priming in the reverse transcription followed by qPCR with the resulting cDNA (GSP 2 step), or gene-specific priming and qRTPCR in a one-step reaction (GSP 1 step). RNA from eleven samples was divided into the three methods and RNA levels for two genes were measured: β-actin (C), HUMSPB (D). The median Ct value obtained with each method is indicated by the solid line.

FIG. 5 is a heatmap showing the relative expression levels of the 10 Marker panel across 239 samples. Red indicates higher expression.

### DETAILED DESCRIPTION

A Biomarker is any indicia of the level of expression of an indicated Marker gene. The indicia can be direct or indirect and measure over- or under-expression of the gene given the physiologic parameters and in comparison to an internal control, normal tissue or another carcinoma. Biomarkers include, without limitation, nucleic acids (both over and under-expression and direct and indirect). Using nucleic acids as Biomarkers can include any method known in the art including, without limitation, measuring DNA amplification, RNA, micro RNA, loss of heterozygosity (LOH), single nucleotide polymorphisms (SNPs, Brookes (1999)), microsatellite DNA, DNA hypo- or hyper-methylation. Using proteins as Biomarkers can include any method known in the art including, without limitation, measuring amount, activity, modifications such as glycosylation, phosphorylation, ADP-ribosylation, ubiquitination, etc., imunohistochemistry (IHC). Other Biomarkers include imaging, cell count and apoptosis Markers.

The indicated genes provided herein are those associated with a particular tumor or tissue type. A Marker gene may be associated with numerous cancer types but provided that the expression of the gene is sufficiently associated with one tumor or tissue type to be identified using the algorithm described herein to be specific for a particular origin, the gene can be used in the claimed invention to determine tissue of origin for a carcinoma of unknown primary origin (CUP). Numerous genes associated with one or more cancers are known in the art. The present invention provides preferred Marker genes and even more preferred Marker gene combinations. These are described herein in detail.

“Origin” as referred to in ‘tissue of origin’ means either the tissue type (lung, colon, etc.) or the histological type (adenocarcinoma, squamous cell carcinoma, etc.) depending on the particular medical circumstances and will be understood by anyone of skill in the art.

A Marker gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

The inventive methods, compositions, articles, and kits of described and claimed in this specification include one or more Marker genes. “Marker” or “Marker gene” is used throughout this specification to refer to genes and gene expression products that correspond with any gene the over- or under-expression of which is associated with a tumor or tissue type. The preferred Marker genes are described in more detail in Tables 1 and 15.

TABLE 1
CUP panel

| | | | |
|---|---|---|---|
| SEQ | | | |
| ID | | Chip | |
| NO: | Name | designation | sequence |

| | | | |
|---|---|---|---|
| 1 | SP-B | 209810_at | gaaaaaccagccactgctttacaggacagggggttgaagctgagccccgcctcacaccc |
| | | | acccccatgcactcaaagattggattttacagctacttgcaattcaaaattcagaagaataaa |
| | | | aaatgggaacatacagaactctaaaagatagacatcagaaattgttaagttaagctttttcaa |
| | | | aaaatcagcaattccccagcgtagtcaagggtggacactgcacgctctggcatgatggga |
| | | | tggcgaccgggcaagctttcttcctcgagatgctctgctgcttgagagctattgctttgttaag |
| | | | atataaaaaggggtttctttttgtctttctgtaaggtggacttccagattttgattgaaagtccta |
| | | | gggtgattctatttctgctgtgatttatctgctgaaagctcagctggggttgtgcaagctaggg |
| | | | acccattcctgtgtaatacaatgtctgcaccaatgct |
| 2 | TTF1 | 211024_s_at | gtgattcaaatgggttttccacgctagggcggggcacagattggagagggctctgtgctga |
| | | | catggctctggactctaaagaccaaacttcactctgggcacactctgccagcaaagagga |
| | | | ctcgcttgtaaataccaggatttttttttttttttgaagggaggacgggagctggggagagga |
| | | | aagagtcttcaacataacccacttgtcactgacacaaaggaagtgccccctccccggcac |
| | | | cctctggccgcctaggctcagcggcgaccgccctccgcgaaaatagtttgtttaatgtgaa |
| | | | cttgtagctgtaaaacgctgtcaaaagttggactaaatgcctagtttttagtaatctgtacatttt |
| | | | gttgtaaaaagaaaaaccactcccagtccccagcccttcacattttttatgggcattgacaaa |
| | | | tctgtatattatttggcagtttggtatttgcggcgtcagtctttttctgttgtaact |
| 3 | DSG3 | 205595_at | ccatcccatagaagtccagcagacaggatttgttaagtgccagactttgtcaggaagtcaa |
| | | | ggagcttctgctttgtccgcctctgggtctgtccagccagctgtttccatccctgaccctctgc |
| | | | agcatggtaactatttagtaacggagacttactcggcttctggttccctcgtgcaaccttcca |
| | | | ctgcaggctttgatccacttctcacacaaaatgtgatagtgacagaaagggtgatctgtccc |
| | | | atttccagtgttcctggcaacctagctggcccaacgcagctacgagggtcacatactatgct |
| | | | ctgtacagaggatccttgctcccgtctaatatgaccagaatgagctggaataccacactgac |
| | | | caaatctggatctttggactaaagtattcaaaatagcatagcaaagctcactgtattgggcta |
| | | | ataatttggcacttattagcttctctcataaactgatcacgattataaattaaatgtttgggttcat |
| | | | accccaaaagcaatatgttgtcactcctaattctcaagtac |
| 4 | HPT1 | 209847_at | ctgcacccacctacttagatatttcatgtgctatagacattagagagatttttcatttttccatga |
| | | | catttttcctctctgcaaatggcttagctacttgtgtttttcccttttggggcaagacagactcatt |
| | | | aaatattctgtacattttttctttatcaaggagatatatcagtgttgtctcatagaactgcctggat |
| | | | tccatttatgttttttctgattccatcctgtgtccccttcatccttgactcctttggtatttcactgaa |
| | | | tttcaaacatttgtc |
| 5 | PSCA | 205319_at | ttcctgaggcacatcctaacgcaagtttgaccatgtatgtttgcaccccttttccccnaaccct |
| | | | gaccttcccatgggccttttccaggattccnaccnggcagatcagttttagtganacanatc |
| | | | cgcntgcagatggcccctccaaccntttntgttgntgtttccatggcccagcattttccaccc |
| | | | ttaaccctgtgttcaggcacttnttcccccaggaagccttccctgcccaccccatttatgaatt |
| | | | gagccaggtttggtccgtggtgtcccccgcacccagcaggggacaggcaatcaggagg |
| | | | gcccagtaaaggctgagatgaagtggactgagtagaactggaggacaagagttgacgtg |
| | | | agttcctgggagtttccagagatg |
| 6 | F5 | 204713_s_at | atcctctacagccagatgtcacagggatacgtctactttcacttggtgctggagaattcanaa |
| | | | gtcaagaacatgctaagcntaagggacccaaggtagaaagagatcaagcagcaaagca |
| | | | caggttctcctggatgaaattactagcacataaagttgggagacacctaagccaagacact |
| | | | ggttctccttccggaatgaggccctgggaggaccttcctagccaagacactggttctccttc |
| | | | cagaatgaggccctggaaggaccctcctagtgatctgttactcttaaaacaaagtaactcat |
| | | | ctaagattttggttgggagatggcatttggcttctgagaaaggtagctatgaaataatccaag |
| | | | atactgatgaagacacagctgttaacaattggctgatcagcccccagaatgcctcacgtgct |
| | | | tggggagaaagcacccctcttgccaacaagcctggaaag |
| 7 | MGB1 | 206378_at | gcagcagcctcaccatgaagttgctgatggtcctcatgctggcggccctctcccagcactg |
| | | | ctacgcaggctctggctgccccttattggagaatgtgatttccaagacaatcaatccacaag |
| | | | tgtctaagactgaatacaaagaacttcttcaagagttcatagacgacaatgccactacaaat |
| | | | gccatagatgaattgaaggaatgttttcttaaccaaacggatgaaactctgagcaatgttga |
| | | | ggtgtttatgcaattaatatatgacagcagtctttgtgatttattttaactttctgcaagacctttg |
| | | | gctcacagaactgcagggtatggtgagaaaccaactacggattgctgcaaaccacacctt |
| | | | ctctttcttatgtctttttact |
| 8 | PDEF | 220192_x_at | gagtggggcccttaaactggattcaaaaaatgctctaaacataggaatggttgaagaggtc |
| | | | ttgcagtcttcagatgaaactaaatctctagaagaggcacaagaatggctaaagcaattcat |
| | | | ccaagggccaccggaagtaattagagctttgaaaaaatctgtttgttcaggcagagagctat |
| | | | atttggaggaagcattacagaacgaaagagatcttttaggaacagtttggggtgggcctgc |
| | | | aaatttagaggctattgctaagaaaggaaaatttaataaataattggtttttcgtgtggatgtac |
| | | | tccaagtaaagctccagtgactaatatgtataaatgttaaatgatattaaatatgaacatcagtt |
| | | | aaaaaaaaaattctttaaggctactattaatatgcagacttacttttaatcatttgaaatctgaac |
| | | | tcatttacctcatttcttgccaattactcccttgggtatttactgcgta |
| 9 | PSA | 204582_s_at | tggtgtaattttgtcctctctgtgtcctggggaatactggccatgcctggagacatatcactca |
| | | | atttctctgaggacacagataggatggggtgtctgtgttatttgtggggtacagagatgaaa |
| | | | gaggggtgggatccacactgagagagtggagagtgacatgtgctggacactgtccatga |
| | | | agcactgagcagaagctggaggcacaacgcaccagacactcacagcaaggatggagct |
| | | | gaaaacataacccactctgtcc |
| 10 | WT1 | 206067_s_at | atagatgtacatacctccttgcacaaatggaggggaattcattttcatcactgggagtgtcctt |
| | | | agtgtataaaaaccatgctggtatatggcttcaagttgtaaaaatgaaagtgactttaaaaga |
| | | | aaataggggatggtccaggatctccactgataagactgtttttaagtaacttaaggacctttg |
| | | | ggtctacaagtatatgtgaaaaaaatgagacttactgggtgaggaaatccattgtttaaagat |
| | | | ggtcgtgtgtgtgtgtgtgtgtgtgtgtgtgttgtgttgtgttttgttttttaagggagggaattta |
| | | | ttatttaccgttgcttgaaattactgtgtaaatatatgtctgataatgatttgctctttgacaactaa |
| | | | aattaggactgtataagtactagatgcatcactgggtgttgatcttacaagat |

The present invention provides a method of diagnosing pancreatic cancers. The present invention thus provides methods for determining the direction of therapy by identifying pancreatic cancers potentially early enough to avoid resection thus allowing for chemotherapeutic regimens.

The present invention further provides composition containing at least one isolated sequence selected from SEQ ID NOs: 39-41 and 43-45. The present invention further provides kits for conducting an assay according to the methods provided herein and further containing Biomarker detection reagents.

The present invention further provides methods for measuring gene expression by generating the amplicons of SEQ ID NOs: 42 and 46 to determine gene expression and comparing levels of at least one of these amplicons to normal tissue gene expression to diagnose pancreatic cancer.

The present invention further provides microarrays or gene chips for performing the methods described herein.

The present invention further provides diagnostic/prognostic portfolios containing isolated nucleic acid sequences, their complements, or portions thereof of a combination of genes as described herein where the combination is sufficient to measure or characterize gene expression in a biological sample having metastatic cells relative to cells from different carcinomas or normal tissue.

Any method described in the present invention can further include measuring expression of at least one gene constitutively expressed in the sample.

Preferably the Markers for pancreatic cancer are coagulation factor V (F5), prostate stem cell antigen (PSCA), integrin, β6 (ITGB6), kallikrein 10 (KLK10), claudin 18 (CLDN18), trio isoform (TR10), and hypothetical protein FLJ22041 similar to FK506 binding proteins (FKBP10). Preferably, Biomarkers for F5 and PSCA are measured together. Biomarkers for ITGB6, KLK10, CLDN18, TR10, and FKBP10 can be measured in addition to or in place of F5 and/or PSCA. F5 is described for instance by 20040076955; 20040005563; and WO2004031412. PSCA is described for instance by WO1998040403; 20030232350; and WO2004063355. ITGB6 is described for instance by WO2004018999; and 6339148. KLK10 is described for instance by WO2004077060; and 20030235820. CLDN18 is described for instance by WO2004063355; and WO2005005601. TR10 is described for instance by 20020055627. FKBP10 is described for instance by WO2000055320.

The invention further provides a method for providing a prognosis by determining the presence of pancreatic cancer according to the methods described herein and identifying the corresponding prognosis therefor.

The invention further provides a method for finding Biomarkers comprising determining the expression level of a Marker gene in a particular metastasis, measuring a Biomarker for the Marker gene to determine expression thereof, analyzing the expression of the Marker gene according to the methods described herein and determining if the Marker gene is effectively specific for pancreatic cancer.

The invention further provides compositions comprising at least one isolated sequence selected from SEQ ID NOs: 39-46.

The invention further provides kits, articles, microarrays or gene chip, diagnostic/prognostic portfolios for conducting the assays described herein and patient reports for reporting the results obtained by the present methods.

The mere presence or absence of particular nucleic acid sequences in a tissue sample has only rarely been found to have diagnostic or prognostic value. Information about the expression of various proteins, peptides or mRNA, on the other hand, is increasingly viewed as important. The mere presence of nucleic acid sequences having the potential to express proteins, peptides, or mRNA (such sequences referred to as “genes”) within the genome by itself is not determinative of whether a protein, peptide, or mRNA is expressed in a given cell. Whether or not a given gene capable of expressing proteins, peptides, or mRNA does so and to what extent such expression occurs, if at all, is determined by a variety of complex factors. Irrespective of difficulties in understanding and assessing these factors, assaying gene expression can provide useful information about the occurrence of important events such as tumorogenesis, metastasis, apoptosis, and other clinically relevant phenomena. Relative indications of the degree to which genes are active or inactive can be found in gene expression profiles. The gene expression profiles of this invention are used to provide a diagnosis and treat patients for CUP.

In the above methods, the sample can be prepared by any method known in the art including, but not limited to, bulk tissue preparation and laser capture microdissection. The bulk tissue preparation can be obtained for instance from a biopsy or a surgical specimen.

In the above methods, the gene expression measuring can also include measuring the expression level of at least one gene constitutively expressed in the sample.

In the above methods, the specificity is preferably at least about 40% and the sensitivity at least at least about 80%.

In the above methods, the pre-determined cut-off levels are at least about 1.5-fold over- or under-expression in the sample relative to benign cells or normal tissue.

In the above methods, the pre-determined cut-off levels have at least a statistically significant p-value over-expression in the sample having metastatic cells relative to benign cells or normal tissue, preferably the p-value is less than 0.05.

In the above methods, gene expression can be measured by any method known in the art, including, without limitation on a microarray or gene chip, nucleic acid amplification conducted by polymerase chain reaction (PCR) such as reverse transcription polymerase chain reaction (RT-PCR), measuring or detecting a protein encoded by the gene such as by an antibody specific to the protein or by measuring a characteristic of the gene such as DNA amplification, methylation, mutation and allelic variation. The microarray can be for instance, a cDNA array or an oligonucleotide array. All these methods and can further contain one or more internal control reagents.

The present invention provides a method of generating a pancreatic cancer prognostic patient report by determining the results of any one of the methods described herein and preparing a report displaying the results and patient reports generated thereby. The report can further contain an assessment of patient outcome and/or probability of risk relative to the patient population.

Sample preparation requires the collection of patient samples. Patient samples used in the inventive method are those that are suspected of containing diseased cells such as cells taken from a nodule in a fine needle aspirate (FNA) of tissue. Bulk tissue preparation obtained from a biopsy or a surgical specimen and laser capture microdissection are also suitable for use. Laser Capture Microdissection (LCM) technology is one way to select the cells to be studied, minimizing variability caused by cell type heterogeneity. Consequently, moderate or small changes in Marker gene expression between normal or benign and cancerous cells can be readily detected. Samples can also comprise circulating epithelial cells extracted from peripheral blood. These can be obtained according to a number of methods but the most preferred method is the magnetic separation technique described in U.S. Pat. No. 6,136,182. Once the sample containing the cells of interest has been obtained, a gene expression profile is obtained using a Biomarker, for genes in the appropriate portfolios.

Preferred methods for establishing gene expression profiles include determining the amount of RNA that is produced by a gene that can code for a protein or peptide. This is accomplished by reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in for instance, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. Nos. 6,271,002; 6,218,122; 6,218,114; and 6,004,755.

Analysis of the expression levels is conducted by comparing such signal intensities. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples.

The selection can be based on statistical tests that produce ranked lists related to the evidence of significance for each gene's differential expression between factors related to the tumor's original site of origin. Examples of such tests include ANOVA and Kruskal-Wallis. The rankings can be used as weightings in a model designed to interpret the summation of such weights, up to a cutoff, as the preponderance of evidence in favor of one class over another. Previous evidence as described in the literature may also be used to adjust the weightings.

A preferred embodiment is to normalize each measurement by identifying a stable control set and scaling this set to zero variance across all samples. This control set is defined as any single endogenous transcript or set of endogenous transcripts affected by systematic error in the assay, and not known to change independently of this error. All Markers are adjusted by the sample specific factor that generates zero variance for any descriptive statistic of the control set, such as mean or median, or for a direct measurement. Alternatively, if the premise of variation of controls related only to systematic error is not true, yet the resulting classification error is less when normalization is performed, the control set will still be used as stated. Non-endogenous spike controls could also be helpful, but are not preferred.

Gene expression profiles can be displayed in a number of ways. The most common is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio less than one (down-regulation) appears in the blue portion of the spectrum while a ratio greater than one (up-regulation) appears in the red portion of the spectrum. Commercially available computer software programs are available to display such data including “Genespring” (Silicon Genetics, Inc.) and “Discovery” and “Infer” (Partek, Inc.)

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

Modulated genes used in the methods of the invention are described in the Examples. The genes that are differentially expressed are either up regulated or down regulated in patients with carcinoma of a particular origin relative to those with carcinomas from different origins. Up regulation and down regulation are relative terms meaning that a detectable difference (beyond the contribution of noise in the system used to measure it) is found in the amount of expression of the genes relative to some baseline. In this case, the baseline is determined based on the algorithm. The genes of interest in the diseased cells are then either up regulated or down regulated relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis may include the determination of disease/status issues such as determining the likelihood of relapse, type of therapy and therapy monitoring. In therapy monitoring, clinical judgments are made regarding the effect of a given course of therapy by comparing the expression of genes over time to determine whether the gene expression profiles have changed or are changing to patterns more consistent with normal tissue.

Genes can be grouped so that information obtained about the set of genes in the group provides a sound basis for making a clinically relevant judgment such as a diagnosis, prognosis, or treatment choice. These sets of genes make up the portfolios of the invention. As with most diagnostic Markers, it is often desirable to use the fewest number of Markers sufficient to make a correct medical judgment. This prevents a delay in treatment pending further analysis as well unproductive use of time and resources.

One method of establishing gene expression portfolios is through the use of optimization algorithms such as the mean variance algorithm widely used in establishing stock portfolios. This method is described in detail in 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is preferred. Markowitz (1952). Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional Markers such as serum protein Markers (e.g., Cancer Antigen 27.29 (“CA 27.29”)). A range of such Markers exists including such analytes as CA 27.29. In one such method, blood is periodically taken from a treated patient and then subjected to an enzyme immunoassay for one of the serum Markers described above. When the concentration of the Marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Kits made according to the invention include formatted assays for determining the gene expression profiles. These can include all or some of the materials needed to conduct the assays such as reagents and instructions and a medium through which Biomarkers are assayed.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

The following examples are provided to illustrate but not limit the claimed invention. All references cited herein are hereby incorporated herein by reference.

### EXAMPLE 1

### Materials and Methods

### Pancreatic Cancer Markers Gene Discovery.

RNA was isolated from pancreatic tumor, normal pancreatic, lung, colon, breast and ovarian tissues using Trizol. The RNA was then used to generate amplified, labeled RNA (Lipshutz et al. (1999)) which was then hybridized onto Affymetrix U133A arrays. The data were then analyzed in two ways.

In the first method, this dataset was filtered to retain only those genes with at least two present calls across the entire dataset. This filtering left 14,547 genes. 2,736 genes were determined to be overexpressed in pancreatic cancer versus normal pancreas with a p value of less than 0.05. Forty five genes of the 2,736 were also overexpressed by at least two-fold compared to the maximum intensity found from lung and colon tissues. Finally, six probe sets were found which were overexpressed by at least two-fold compared to the maximum intensity found from lung, colon, breast, and ovarian tissues.

In the second method, this dataset was filtered to retain only those genes with no more than two present calls in breast, colon, lung, and ovarian tissues. This filtering left 4,654 genes. 160 genes of the 4,654 genes were found to have at least two present calls in the pancreatic tissues (normal and cancer). Finally, eight probe sets were selected which showed the greatest differential expression between pancreatic cancer and normal tissues.

### Tissue Samples.

A total of 260 FFPE metastasis and primary tissues were acquired from a variety of commercial vendors. The samples tested included: 30 breast metastasis, 30 colorectal metastasis, 56 lung metastasis, 49 ovarian metastasis 43 pancreas metastasis, 18 prostate primary and 2 prostate metastases and 32 other origins (6 stomach, 6 kidney, 3 larynx, 2 liver, 1 esophagus, 1 pharynx, 1 bile duct, 1 pleura, 3 bladder, 5 melanoma, 3 lymphoma).

### RNA Extraction.

RNA isolation from paraffin tissue sections was based on the methods and reagents described in the High Pure RNA Paraffin Kit manual (Roche) with the following modifications. Paraffin embedded tissue samples were sectioned according to size of the embedded metastasis (2-5 mm=9×10 μm, 6-8 mm=6×10 μm, 8-≧10 mm=3×10 μm), and placed in RNase/DNase 1.5 ml Eppendorf tubes. Sections were deparaffinized by incubation in 1 ml of xylene for 2-5 min at room temperature following a 10-20 second vortex. Tubes were then centrifuged and supernatant was removed and the deparaffinization step was repeated. After supernatant was removed, 1 ml of ethanol was added and sample was vortexed for 1 minute, centrifuged and supernatant removed. This process was repeated one additional time. Residual ethanol was removed and the pellet was dried in a 55° C. oven for 5-10 minutes and resuspended in 100 μl of tissue lysis buffer, 16 μl 10% SDS and 80 μl Proteinase K. Samples were vortexed and incubated in a thermomixer set at 400 rpm for 2 hours at 55° C. 325 μl binding buffer and 325 μl ethanol was added to each sample that was then mixed, centrifuged and the supernatant was added onto the filter column. Filter column along with collection tube were centrifuged for 1 minute at 8000 rpm and flow through was discarded. A series of sequential washes were performed (500 μl Wash Buffer I→500 μl Wash Buffer II→300 μl Wash Buffer II) in which each solution was added to the column, centrifuged and flow through discarded. Column was then centrifuged at maximum speed for 2 minutes, placed in a fresh 1.5 ml tube and 90 μl of elution buffer was added. RNA was obtained after a 1 minute incubation at room temperature followed by a 1 minute centrifugation at 8000 rpm. Sample was DNase treated with the addition of 10 μl DNase incubation buffer, 2 μl of DNase I and incubated for 30 minutes at 37° C. DNase was inactivated following the addition of 20 μl of tissue lysis buffer, 18 μl 10% SDS and 40 μl Proteinase K. Again, 325 μl binding buffer and 325 μl ethanol was added to each sample that was then mixed, centrifuged and supernatant was added onto the filter column. Sequential washes and elution of RNA proceeded as stated above with the exception of 50 μl of elution buffer being used to elute the RNA. To eliminate glass fiber contamination carried over from the column RNA was centrifuged for 2 minutes at full speed and supernatant was removed into a fresh 1.5 ml Eppendorf tube. Samples were quantified by OD 260/280 readings obtained by a spectrophotometer and samples were diluted to 50 ng/μl. The isolated RNA was stored in Rnase-free water at −80° C. until use.

### TaqMan Primer and Probe Design.

Appropriate mRNA reference sequence accession numbers in conjunction with Oligo 6.0 were used to develop TaqMan® CUP assays (lung Markers: human surfactant, pulmonary-associated protein B (HUMPSPBA), thyroid transcription factor 1 (TTF1), desmoglein 3 (DSG3), colorectal Marker: cadherin 17 (CDH17), breast Markers: mammaglobin (MG), prostate-derived ets transcription factor (PDEF), ovarian Marker: wilms tumor 1 (WT1), pancreas Markers: prostate stem cell antigen (PSCA), coagulation factor V (F5), prostate Marker kallikrein 3 (KLK3)) and housekeeping assays beta actin (β-Actin), hydroxymethylbilane synthase (PBGD). Primers and hydrolysis probes for each assay are listed in Table 2. Genomic DNA amplification was excluded by designing assays around exon-intron splicing sites. Hydrolysis probes were labeled at the 5′ nucleotide with FAM as the reporter dye and at 3′ nucleotide with BHQ1-TT as the internal quenching dye.

### Quantitative Real-Time Polymerase Chain Reaction.

Quantitation of gene-specific RNA was carried out in a 384 well plate on the ABI Prism 7900HT sequence detection system (Applied Biosystems). For each thermo-cycler run calibrators and standard curves were amplified. Calibrators for each Marker consisted of target gene in vitro transcripts that were diluted in carrier RNA from rat kidney at 1×10⁵ copies. Standard curves for housekeeping Markers consisted of target gene in vitro transcripts that were serially diluted in carrier RNA from rat kidney at 1×10⁷, 1×10⁵ and 1×10³ copies. No target controls were also included in each assay run to ensure a lack of environmental contamination. All samples and controls were run in duplicate. qRTPCR was performed with general laboratory use reagents in a 10 μl reaction containing: RT-PCR Buffer (50 nM Bicine/KOH pH 8.2, 115 nM KAc, 8% glycerol, 2.5 mM MgCl₂, 3.5 mM MnSO₄, 0.5 mM each of dCTP, dATP, dGTP and dTTP), Additives (2 mM Tris-Cl pH 8, 0.2 mM Albumin Bovine, 150 mM Trehalose, 0.002% Tween 20), Enzyme Mix (2U Tth (Roche), 0.4 mg/μl Ab TP6-25), Primer and Probe Mix (0.2 μM Probe, 0.5 μM Primers). The following cycling parameters were followed: 1 cycle at 95° C. for 1 minute; 1 cycle at 55° C. for 2 minutes; Ramp 5%; 1 cycle at 70° C. for 2 minutes; and 40 cycles of 95° C. for 15 seconds, 58° C. for 30 seconds. After the PCR reaction was completed, baseline and threshold values were set in the ABI 7900HT Prism software and calculated Ct values were exported to Microsoft Excel.

One-Step vs. Two-Step Reaction.

First strand synthesis was carried out using either 100 ng of random hexamers or gene specific primers per reaction. In the first step, 11.5 μl of Mix-1 (primers and 1 ug of total RNA) was heated to 65° C. for 5 minutes and then chilled on ice. 8.5 μl of Mix-2 (1× Buffer, 0.01 mM DTT, 0.5 mM each dNTP's, 0.25 U/μl RNasin®, 10U/μl Superscript III) was added to Mix-1 and incubated at 50° C. for 60 minutes followed by 95° C. for 5 minutes. The cDNA was stored at −20° C. until ready for use. qRTPCR for the second step of the two-step reaction was performed as stated above with the following cycling parameters: 1 cycle at 95° C. for 1 minute; 40 cycles of 95° C. for 15 seconds, 58° C. for 30 seconds. qRTPCR for the one-step reaction was performed exactly as stated in the preceding paragraph. Both the one-step and two-step reactions were performed on 100 ng of template (RNA/cDNA). After the PCR reaction was completed, baseline and threshold values were set in the ABI 7900HT Prism software and calculated Ct values were exported to Microsoft Excel.

### Generation of a Heatmap.

For each sample, a ΔCt was calculated by taking the mean Ct of each CUP Marker and subtracting the mean Ct of an average of the housekeeping Markers (ΔCt=Ct(CUP Marker)−Ct(Ave. HK Marker)). The minimal ΔCt for each tissue of origin Marker set (lung, breast, prostate, colon, ovarian and pancreas) was determined for each sample. The tissue of origin with the overall minimal ΔCt was scored one and all other tissue of origins scored zero. Data were sorted according to pathological diagnosis. Partek Pro was populated with the modified feasibility data and an intensity plot was generated.

### Results.

### Discovery of Novel Pancreatic Tumor of Origin and Cancer Status Markers.

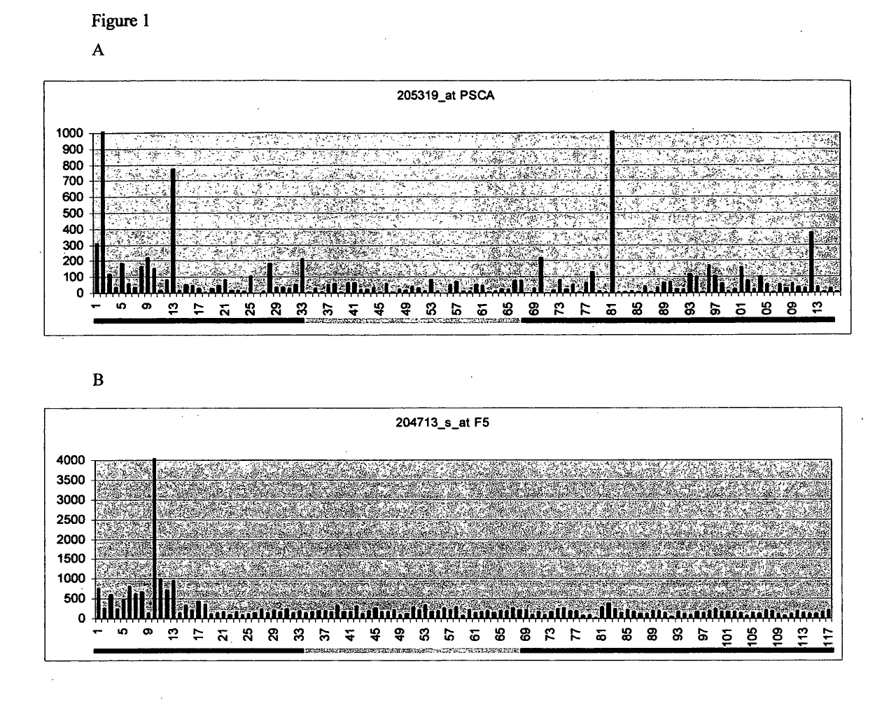

First, five pancreas Marker candidates were analyzed: prostate stem cell antigen (PSCA), serine proteinase inhibitor, clade A member 1 (SERPINA1), cytokeratin 7 (KRT7), matrix metalloprotease 11 (MMP11), and mucin4 (MUC4) (Varadhachary et al (2004); Fukushima et al. (2004); Argani et al. (2001); Jones et al. (2004); Prasad et al. (2005); and Moniaux et al. (2004)) using DNA microarrays and a panel of 13 pancreatic ductal adenocarcinomas, five normal pancreas tissues, and 98 samples from breast, colorectal, lung, and ovarian tumors. Only PSCA demonstrated moderate sensitivity (six out of thirteen or 46% of pancreatic tumors were detected) at a high specificity (91 out of 98 or 93% were correctly identified as not being of pancreatic origin) (FIG. 1A). In contrast, KRT7, SERPINA1, MMP11, and MUC4 demonstrated sensitivities of 38%, 31%, 85%, and 31%, respectively, at specificities of 66%, 91%, 82%, and 81%, respectively. These data were in good agreement with qRTPCR performed on 27 metastases of pancreatic origin and 39 metastases of non-pancreatic origin for all Markers except for MMP11 which showed poorer sensitivity and specificity with qRTPCR and the metastases. In conclusion, the microarray data on snap frozen, primary tissue serves as a good indicator of the ability of the Marker to identify a FFPE metastasis as being pancreatic in origin using qRTPCR but that additional Markers may be useful for optimal performance.

Because pancreatic ductal adenocarcinoma develops from ductal epithelial cells that comprise only a small percentage of all pancreatic cells (with acinar cells and islet cells comprising the majority) and because pancreatic adenocarcinoma tissues contain a significant amount of adjacent normal tissue (Prasad et al. (2005); and Ishikawa et al. (2005)), it has been difficult to identify pancreatic cancer Markers (i.e., upregulated in cancer) which would also differentiate this organ from the organs. For use in a CUP panel such differentiation is necessary. The first query method (see Materials and Methods) returned six probe sets: coagulation factor V (F5), a hypothetical protein FLJ22041 similar to FK506 binding proteins (FKBP10), β6 integrin (ITGB6), transglutaminase 2 (TGM2), heterogeneous nuclear ribonucleoprotein A0 (HNRP0), and BAX delta (BAX). The second query method (see Materials and Methods) returned eight probe sets: F5, TGM2, paired-like homeodomain transcription factor 1 (PITX1), trio isoform mRNA (TRIO), mRNA for p73H (p73), an unknown protein for MGC:10264 (SCD), and two probe sets for claudin18. F5 and TGM2 were present in both query results and, of the two, F5 looked the most promising (FIG. 1B).

Optimization of Sample Prep and qRTPCR Using FFPE Tissues.

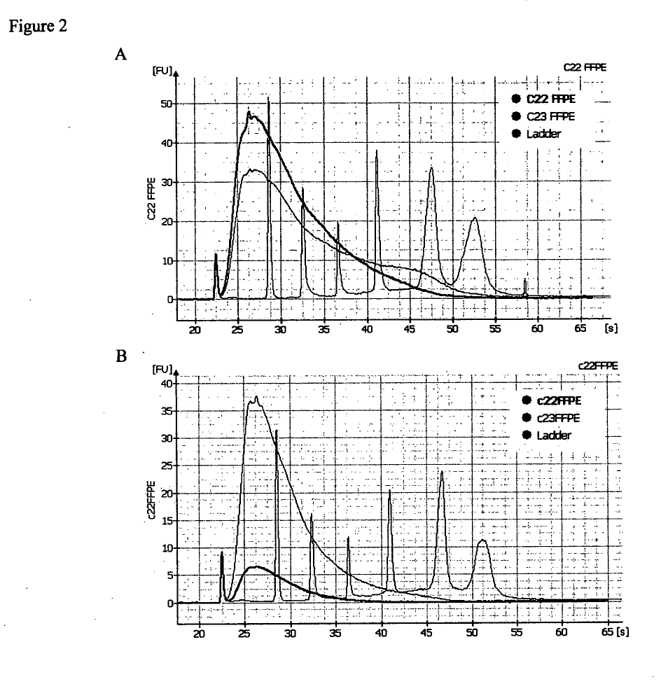

Next the RNA isolation and qRTPCR methods were optimized using fixed tissues before examining Marker panel performance. First the effect of reducing the proteinase K incubation time from sixteen hours to 3 hours was analyzed. There was no effect on yield. However, some samples showed longer fragments of RNA when the shorter proteinase K step was used (FIG. 2). For example, when RNA was isolated from a one year old block (C22), there was no observed difference in the electropherograms. However, when RNA was isolated from a five year old block (C23), a larger fraction of higher molecular weight RNAs was observed, as assessed by the hump in the shoulder, when the shorter proteinase K digest was used. This trend generally held when other samples were processed, regardless of the organ of origin for the FFPE metastasis. In conclusion, shortening the proteinase K digestion time does not sacrifice RNA yields and may aid in isolating longer, less degraded RNA.

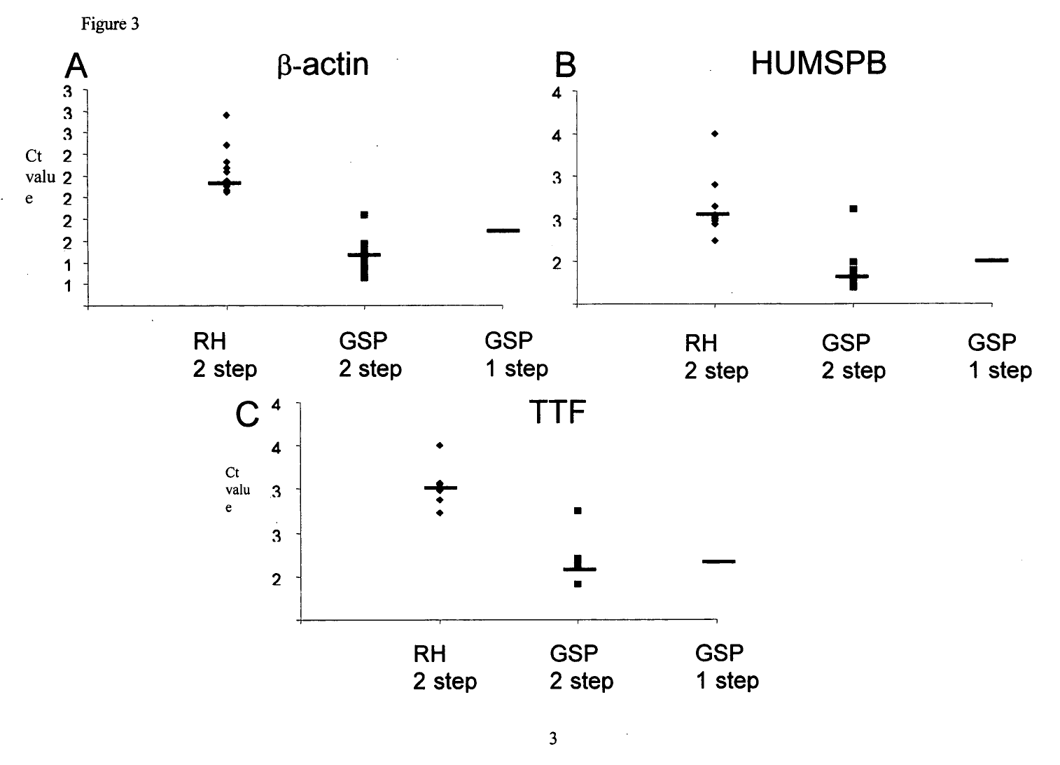

Next, three different methods of reverse transcription were compared: reverse transcription with random hexamers followed by qPCR (two step), reverse transcription with a gene-specific primer followed by qPCR (two step), and a one-step qRTPCR using gene-specific primers. RNA was isolated from eleven metastases and compared Ct values across the three methods for β-actin, human surfactant protein B (HUMSPB), and thyroid transcription factor (TTF) (FIG. 3). There were statistically significant differences (p<0.001) for all comparisons. For all three genes, the reverse transcription with random hexamers followed by qPCR (two step reaction) gave the highest Ct values while the reverse transcription with a gene-specific primer followed by qPCR (two step reaction) gave slightly (but statistically significant) lower Ct values than the corresponding 1 step reaction. However, the 2 step RTPCR with gene-specific primers had a longer reverse transcription step. When HUMSPB and TTF Ct values were normalized to the corresponding β-actin value for each sample, there were no differences in the normalized Ct values across the three methods. In conclusion, optimization of the RTPCR reaction conditions can generate lower Ct values, which may help in analyzing older paraffin blocks (Cronin et al (2004)), and a one step RTPCR reaction with gene-specific primers can generate Ct values comparable to those generated in the corresponding two step reaction.

Diagnostic Performance of a CUP qRTPCR Assay.

Next 12 qRTPCR reactions (10 Markers and two housekeeping genes) were performed on 239 FFPE metastases. The Markers used for the assay are shown in Table 2. The lung Markers were human surfactant pulmonary-associated protein B (HUMPSPB), thyroid transcription factor 1 (TTF1), and desmoglein 3 (DSG3). The colorectal Marker was cadherin 17 (CDH17). The breast Markers were mammaglobin (MG) and prostate-derived Ets transcription factor (PDEF). The ovarian Marker was Wilms tumor 1 (WT1). The pancreas Markers were prostate stem cell antigen (PSCA) and coagulation factor V (F5), and the prostate Marker was kallikrein 3 (KLK3). For gene descriptions, see Table 15.

TABLE 2
Primer and probe sequences, accession numbers, and amplicon lengths.

| | SEQ | | | SEQ |
|---|---|---|---|---|
| | ID | | | ID |
| Target | NO | Sequence (5′-3′) | Description | NO |
| SP-B | 59 | cacagccccgacctttgatga | Forward primer | 11 |
| | | ggtcccagagcccgtctca | Reverse primer | 12 |
| | | agctgtccagctgcaaaggaaaagcc | Probe* | 13 |
| | | cacagccccgacctttgatgagaactcagctgtccagctgcaaaggaaaagc | Amplicon | 14 |
| | | caagtgagacgggctctgggacc | | |
| TTF1 | 60 | ccaacccagacccgcgc | Forward primer | 15 |
| | | cgcccatgccgctcatgttca | Reverse primer | 16 |
| | | cccgccatctcccgcttcatg | Probe* | 17 |
| | | caacccagacccgcgcttccccgccatctcccgcttcatgggcccggcgagc | Amplicon | 18 |
| | | ggcatgaacatgagcggcatgggcg | | |
| DSG3 | 61 | gcagagaaggagaagataactcaa | Forward primer | 19 |
| | | actccagagattcggtaggtga | Reverse primer | 20 |
| | | attgccaagattacttcagattacca | Probe* | 21 |
| | | gcagagaaggagaagataactcaaaaagaaacccaattgccaagattacttc | Amplicon | 22 |
| | | agattaccaagcaacccagaaaatcacctaccgaatctctggagt | | |
| CDH17 | 62 | tccctcggcagtggaagctta | Forward primer | 23 |
| | | tcctcaaactctgtgtgcctggta | Reverse primer | 24 |
| | | ccaaaatcaatggtactcatgcccgactg | Probe* | 25 |
| | | tccctcggcagtggaagcttacaaaacgactgggaagtttccaaaatcaatg | Amplicon | 26 |
| | | gtactcatgcccgactgtctaccaggcacacagagtttgagga | | |
| MG | 63 | agttgctgatggtcctcatgc | Forward primer | 27 |
| | | cacttgtggattgattgtcttgga | Reverse primer | 28 |
| | | ccctctcccagcactgctacgca | Probe* | 28 |
| | | agttgctgatggtcctcatgctggcggccctctcccagcactgctacgcagg | Amplicon | 30 |
| | | ctctggctgccccttattggagaatgtgatttccaagacaatcaatccacaa | | |
| | | gtg | | |
| PDEF | 64 | cgcccacctggacatctgga | Forward primer | 31 |
| | | cactggtcgaggcacagtagtga | Reverse primer | 32 |
| | | gtcagcggcctggatgaaagagcgg | Probe* | 33 |
| | | cgcccacctggacatctggaagtcagcggcctggatgaaagagcggacttca | Amplicon | 34 |
| | | cctggggcgattcactactgtgcctcgaccagtg | | |
| WT1 | 65 | gcggagcccaatacagaatacac | Forward primer | 35 |
| | | cggggctactccaggcaca | Reverse primer | 36 |
| | | tcagaggcattcaggatgtgcgacg | Probe* | 37 |
| | | gcggagcccaatacagaatacacacgcacggtgtcttcagaggcattcagga | Amplicon | 38 |
| | | tgtgcgacgtgtgcctggagtagccccg | | |
| PSCA | 66 | ctgttgatggcaggcttggc | Forward primer | 39 |
| | | ttgctcacctgggctttgca | Reverse primer | 40 |
| | | gcagccaggcactgccctgct | Probe* | 41 |
| | | ctgttgatggcaggcttggccctgcagccaggcactgccctgctgtgctact | Amplicon | 42 |
| | | cctgcaaagcccaggtgagcaa | | |
| F5 | 67 | tgaagaaatatcctgggattattca | Forward primer | 43 |
| | | tatgtggtatcttctggaatatcatca | Reverse primer | 44 |
| | | acaaagggaaacagatattgaagactc | Probe* | 45 |
| | | tgaagaaatatcctgggattattcagaatttgtacaaagggaaacagatatt | Amplicon | 46 |
| | | gaagactctgatgatattccagaagataccacata | | |
| KLK3 | 68 | cccccagtgggtcctcaca | Forward primer | 47 |
| | | aggatgaaacaagctgtgccga | Reverse primer | 48 |
| | | caggaacaaaagcgtgatcttgctgg | Probe* | 49 |
| | | cccccagtgggtcctcacagctgcccactgcatcaggaacaaaagcgtgatc | Amplicon | 50 |
| | | ttgctgggtcggcacagcttgtttcatcct | | |
| B actin | 69 | gccctgaggcactcttcca | Forward primer | 51 |
| | | cggatgtccacgtcacacttca | Reverse primer | 52 |
| | | cttccttcctgggcatggagtcctg | Probe* | 53 |
| | | gccctgaggcactcttccagccttccttcctgggcatggagtcctgtggcat | Amplicon | 54 |
| | | ccacgaaactaccttcaactccatcatgaagtgtgacgtggacatccg | | |
| PBGD | 70 | ccacacacagcctactttccaa | Forward primer | 55 |
| | | tacccacgcgaatcactctca | Reverse primer | 56 |
| | | aacggcaatgcggctgcaacggcggaa | Probe* | 57 |
| | | ccacacacagcctactttccaagcggagccatgtctggtaacggcaatgcgg | Amplicon | 58 |
| | | ctgaacggcggaagaaaacagcccaaagatgagagtgattcgcgtgggta | | |

*Probes are 5′FAM-3′BHQ1-TT

Analysis of the normalized Ct values in a heat map revealed the high specificity of the breast and prostate Markers, moderate specificity of the colon, lung, and ovarian, and somewhat lower specificity of the pancreas Markers. Combining the normalized qRTPCR data with computational refinement improves the performance of the Marker panel. Results were obtained from the combined normalized qRTPCR data with the algorithm and the accuracy of the qRTPCR assay was determined.

### Discussion.

In this example, microarray-based expression profiling was used on primary tumors to identify candidate Markers for use with metastases. The fact that primary tumors can be used to discover tumor of origin Markers for metastases is consistent with several recent findings. For example, Weigelt and colleagues have shown that gene expression profiles of primary breast tumors are maintained in distant metastases. Weigelt et al. (2003). Italiano and coworkers found that EGFR status, as assessed by IHC, was similar in 80 primary colorectal tumors and the 80 related metastases. Italiano et al. (2005). Only five of the 80 showed discordance in EGFR status. Italiano et al. (2005). Backus and colleagues identified putative Markers for detecting breast cancer metastasis using a genome-wide gene expression analysis of breast and other tissues and demonstrated that mammaglobin and CK19 detected clinically actionable metastasis in breast sentinel lymph nodes with 90% sensitivity and 94% specificity. Backus et al. (2005).

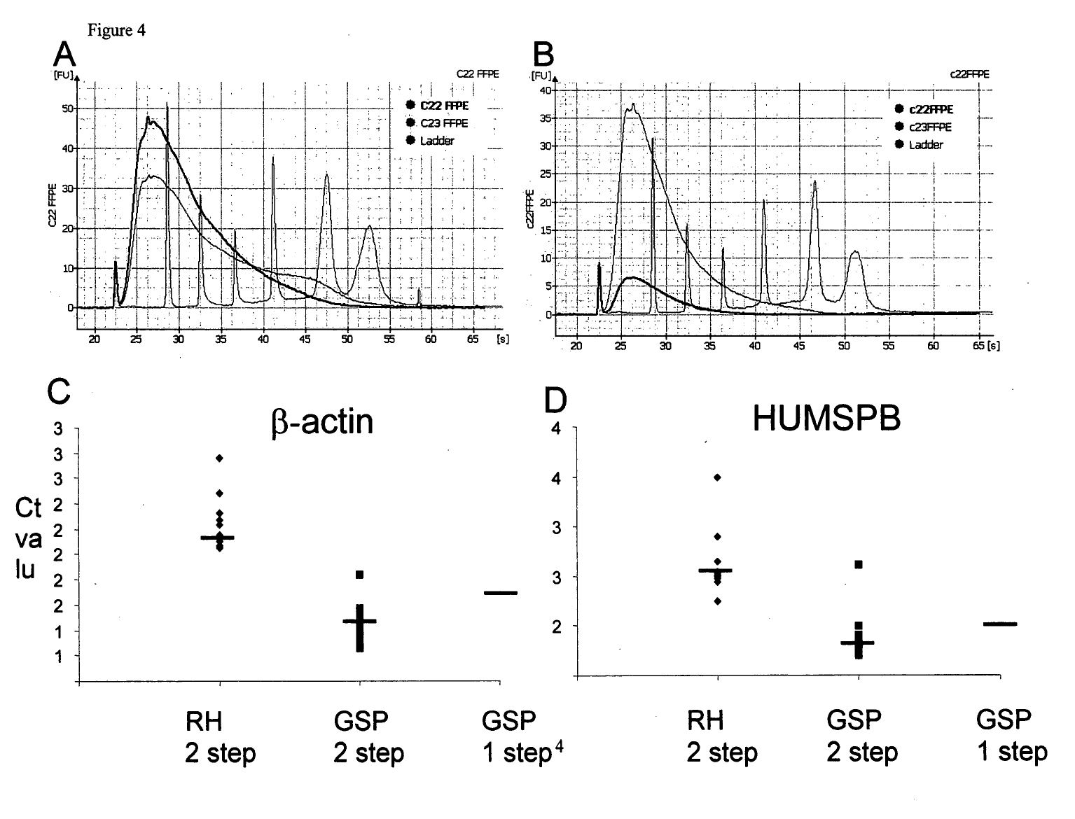

The microarray-based studies with primary tissue confirmed the specificity and sensitivity of known Markers. As a result, with the exception of F5, all of the Markers used have high specificity for the tissues studied here. Argani et al (2001; Backus et al. (2005); Cunha et al. (2005); Borgono et al. (2004); McCarthy et al. (2003); Hwang et al. (2004); Fleming et al. (2000); Nakamura et al. (2002); and Khoor et al. (1997). A recent study determined that, using IHC, PSCA is overexpressed in prostate cancer metastases. Lam et al. (2005). Dennis et al. (2002) also demonstrated that PSCA could be used as a tumor of origin Marker for pancreas and prostate. As shown herein, strong expression of PSCA is found in some prostate tissues at the RNA level but, because by including PSA in the assay, one can now segregate prostate and pancreatic cancers. A novel finding of this study was the use of F5 as a complementary (to PSCA) Marker for pancreatic tissue of origin. In both the microarray data set with primary tissue and the qRTPCR data set with FFPE metastases, F5 was found to complement PSCA (FIG. 4 and Table 3)

TABLE 3
feasibility data

| | Breast | Colon | Lung | Other | Ovary | Pancreas | Prostate | Total |
|---|---|---|---|---|---|---|---|---|
| Total tested | 30 | 30 | 56 | 32 | 49 | 43 | 20 | 260 |
| #Correct | 22 | 27 | 45 | 16 | 43 | 31 | 20 | 204 |
| #Other/No test | 1 | 1 | 3 | n/a | 1 | 4 | 0 | 10 |
| #Incorrect | 7 | 2 | 8 | 16 | 5 | 8 | 0 | 46 |
| % Tested | 96.67 | 96.67 | 94.64 | 100 | 97.96 | 90.70 | 100 | 96.15 |
| % Correct of tested | 75.86 | 193.10 | 84.91 | 0 | 89.58 | 79.49 | 100 | 81.60 |
| Correct of total (%) | 73.33 | 90.00 | 80.36 | 50.00 | 87.76 | 72.09 | 100 | 78.46 |

Previous investigators have generated CUP assays using IHC or microarrays. Su et al. (2001); Ramaswamy et al. (2001); and Bloom et al. (2004). More recently, SAGE has been coupled to a small qRTPCR Marker panel. Dennis (2002); and Buckhaults et al. (2003). This study is the first to combine microarray-based expression profiling with a small panel of qRTPCR assays. Microarray studies with primary tissue identified some, but not all, of the same tissue of origin Markers as those identified previously by SAGE studies. Some studies have demonstrated that a modest agreement between SAGE- and DNA microarray-based profiling data exists and that the correlation improves for genes with higher expression levels. van Ruissen et al. (2005); and Kim (2003). For example, Dennis and colleagues identified PSA, MG, PSCA, and HUMSPB while Buckhaults and coworkers (Dennis et al. (2002)) identified PDEF. Executing the CUP assay using qRTPCR is preferred because it is a robust technology and may have performance advantages over IHC. Al-Mulla et al. (2005); and Haas et al. (2005). As shown herein, the qRTPCR protocol was improved through the use of gene-specific primers in a one-step reaction. This is the first demonstration of the use of gene-specific primers in a one-step qRTPCR reaction with FFPE tissue. Other investigators have either done a two step qRTPCR (cDNA synthesis in one reaction followed by qPCR) or have used random hexamers or truncated gene-specific primers. Abrahamsen et al. (2003); Specht et al. (2001); Godfrey et al. (2000); Cronin et al. (2004); and Mikhitarian et al. (2004).

### EXAMPLE 2

Pancreatic ductal adenocarcinoma develops from ductal epithelial cells that comprise only a small percentage of all pancreatic cells (with acinar and islet cells comprising the majority) in the normal pancreas. Furthermore, pancreatic adenocarcinoma tissues contain a significant amount of adjacent normal tissue. Prasad et al. (2005); and Ishikawa et al. (2005). Because of this the candidate pancreas Markers were enriched for genes elevated in pancreas adenocarcinoma relative to normal pancreas cells. The first query method returned six probe sets: coagulation factor V (F5), a hypothetical protein FLJ22041 similar to FK506 binding proteins (FKBP10), beta 6 integrin (ITGB6), transglutaminase 2 (TGM2), heterogeneous nuclear ribonucleoprotein A0 (HNRP0), and BAX delta (BAX). The second query method (see Materials and Methods section for details) returned eight probe sets: F5, TGM2, paired-like homeodomain transcription factor 1 (PITX1), trio isoform mRNA (TRIO), mRNA for p73H (p73), an unknown protein for MGC:10264 (SCD), and two probe sets for claudin18.

A total of 23 tissue specific Marker candidates were selected for further RT-PCR validation on metastatic carcinoma FFPE tissues by qRT-PCR. Marker candidates were tested on 205 FFPE metastatic carcinomas, from lung, pancreas, colon, breast, ovary, prostate and prostate primary carcinomas. Table 4 provides the gene symbols of the tissue specific Markers selected for RT-PCR validation and also summarizes the results of testing performed with these Markers.

TABLE 4

| | SEQ | ID method | | Marker selection filters | | | |
|---|---|---|---|---|---|---|---|
| Tissue | ID | Micro | | Low exp in | Marker | Tissue cross | Marker |
| type | NOs | array | Lit | corres met tissue | redundancy | reactivity | adequate? |
| Lung | 1/59 | X | X | | | | X |
| | 60 | X | X | | | | X |
| | 61 | | X | | X | | X |
| Pancreas | 66 | | X | | | | X |
| | 67 | X | | | | | X |
| | 71 | X | | | X | | |
| | 72 | X | | X | | | |
| | 73 | | X | | | | |
| | 74 | | X | | | | |
| | 75 | | X | | | | |
| | 76 | | X | | | | |
| Colon | 4/85 | X | X | | | | X |
| | 77 | X | X | | | | |
| | 78 | X | X | | X | | |
| | 79 | X | X | | X | | |
| Prostate | 9/86 | X | X | | | | X |
| | 80 | X | X | | X | | |
| Breast | 63 | X | X | | | | X |
| | 81 | X | X | | | X | |
| | 64 | | X | | | | X |
| Ovarian | 82 | X | X | | | X | |
| | 83 | X | X | | | X | |
| | 65 | X | X | | | | X |

Out of 23 tested Markers, thirteen were rejected based on their cross reactivity, low expression level in the corresponding metastatic tissues, or redundancy. Ten Markers were selected for the final version of assay. The lung Markers were human surfactant pulmonary-associated protein B (HUMPSPB), thyroid transcription factor 1 (TTF1), and desmoglein 3 (DSG3). The pancreas Markers were prostate stem cell antigen (PSCA) and coagulation factor V (F5), and the prostate Marker was kallikrein 3 (KLK3). The colorectal Marker was cadherin 17 (CDH17). Breast Markers were mammaglobin (MG) and prostate-derived Ets transcription factor (PDEF). The ovarian Marker was Wilms tumor 1 (WT1).

Optimization of sample preparation and qRT-PCR using FFPE tissues. Next the RNA isolation and qRTPCR methods were optimized using fixed tissues before examining the performance of the Marker panel. First the effect of reducing the proteinase K incubation time from sixteen hours to 3 hours was analyzed. There was no effect on yield. However, some samples showed longer fragments of RNA when the shorter proteinase K step was used (FIG. 4A, B). For example, when RNA was isolated from a one-year-old block (C22), no difference was observed in the electropherograms. However, when RNA was isolated from a five-year-old block (C23), a larger fraction of higher molecular weight RNAs were observed, as assessed by the hump in the shoulder, when the shorter proteinase K digest was used. This trend generally held when other samples were processed, regardless of the organ of origin for the FFPE metastasis. In conclusion, shortening the proteinase K digestion time does not sacrifice RNA yields and may aid in isolating longer, less degraded RNA.

Next three different methods of reverse transcription were compared: reverse transcription with random hexamers followed by qPCR (two step), reverse transcription with a gene-specific primer followed by qPCR (two step), and a one-step qRTPCR using gene-specific primers. RNA was isolated from eleven metastases and compared Ct values across the three methods for β-actin, HUMSPB (FIG. 4C, D) and TTF. The results showed statistically significant differences (p<0.001) for all comparisons. For both genes, the reverse transcription with random hexamers followed by qPCR (two step reaction) gave the highest Ct values while the reverse transcription with a gene-specific primer followed by qPCR (two-step reaction) gave slightly (but statistically significant) lower Ct values than the corresponding 1 step reaction. However, the two-step RTPCR with gene-specific primers had a longer reverse transcription step. When HUMSPB Ct values were normalized to the corresponding β-actin value for each sample, there were no differences in the normalized Ct values across the three methods. In conclusion, optimization of the RTPCR reaction conditions can generate lower Ct values, which aids in analyzing older paraffin blocks (Cronin et al. (2004)), and a one step RTPCR reaction with gene-specific primers can generate Ct values comparable to those generated in the corresponding two step reaction.

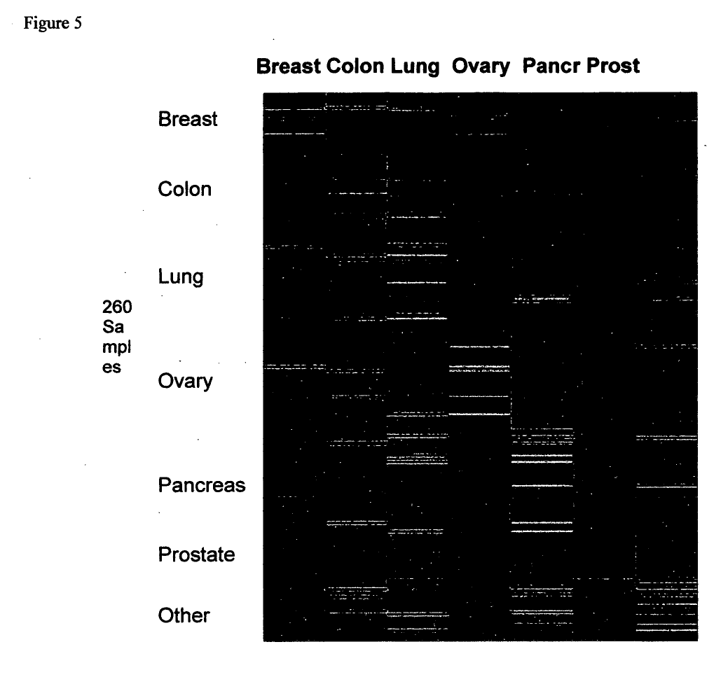

Diagnostic performance of optimized qRTPCR assay. 12 qRTPCR reactions (10 Markers and 2 housekeeping genes) were performed on new set of 260 FFPE metastases. Twenty-one samples gave high Ct values for the housekeeping genes so only 239 were used in a heat map analysis. Analysis of the normalized Ct values in a heat map revealed the high specificity of the breast and prostate Markers, moderate specificity of the colon, lung, and ovarian, and somewhat lower specificity of the pancreas Markers (FIG. 5). Combining the normalized qRTPCR data with computational refinement improves performance of the Marker panel.

Using expression values, normalized to average of expression of two housekeeping genes, an algorithm to predict metastasis tissue of origin was developed by combining the normalized qRTPCR data with the algorithm and determined the accuracy of the qRTPCR assay by performing a leave-one-out-cross-validation test (LOOCV). For the six tissue types included in the assay, it was separately estimated that both the number of false-positive calls, when a sample was wrongly predicted as another tumor type included in the assay (pancreas as colon, for example), and the number of times a sample was not predicted as those included in the assay tissue types (other). Results of the LOOCV are presented on Table 5.

TABLE 5
Tissue of Origin

| Prediction | Breast | Colon | Lung | Ovary | Pancreas | Prostate | Other | Total |
|---|---|---|---|---|---|---|---|---|
| Breast | 22 | 0 | 2 | 1 | 1 | 0 | 0 | |
| Colon | 1 | 27 | 3 | 2 | 4 | 0 | 4 | |
| Lung | 1 | 2 | 45 | 2 | 3 | 0 | 5 | |
| Other | 1 | 1 | 3 | 1 | 4 | 0 | 16 | |
| Ovary | 5 | 0 | 0 | 43 | 0 | 0 | 1 | |
| Pancreas | 0 | 0 | 3 | 0 | 31 | 0 | 6 | |
| Prostate | 0 | 0 | 0 | 0 | 0 | 20 | 0 | |
| Total | 30 | 30 | 56 | 49 | 43 | 20 | 32 | 260 |
| # Correct | 22 | 27 | 45 | 43 | 31 | 20 | 16 | 204 |
| Accuracy | 72.3 | 90.0 | 87.8 | 87.8 | 72.1 | 100.0 | 50.0 | 78.5 |

The tissue of origin was predicted correctly for 204 out of 260 tested samples with an overall accuracy of 78%. A significant proportion of the false positive calls were due to the Markers' cross-reactivity in histologically similar tissues. For example, three squamous cell metastatic carcinomas originated from pharynx, larynx and esophagus were wrongly predicted as lung due to DSG3 expression in these tissues. Positive expression of CDH17 in other than colon GI carcinomas, including stomach and pancreas, caused false classification of 4 out of 6 tested stomach and 3 out of 43 tested pancreatic cancer metastasis as colon.

In addition to a LOOCV test, the data was randomly split into 3 separate pairs of training and test sets. Each split contained approximately 50% of the samples from each class. At 50/50 splits in three separate pairs of training and test sets, assay overall classification accuracies were 77%, 71% and 75%, confirming assay performance stability.

Last, another independent set of 48 FFPE metastatic carcinomas that included metastatic carcinoma of known primary, CUP specimens with a tissue of origin diagnosis rendered by pathological evaluation including IHC, and CUP specimens that remained CUP after IHC testing were tested. The tissue of origin prediction accuracy was estimated separately for each category of samples. Table 6 summarizes the assay results.

TABLE 6

| | Tested | Correct | Accuracy |
|---|---|---|---|
| Known mets | 15 | 11 | 73.3 |
| Resolved CUP | 22 | 17 | 77.3 |
| Unresolved CUP | 11 | | |

The tissue of origin prediction was, with only a few exceptions, consistent with the known primary or tissue of origin diagnosis assessed by clinical/pathological evaluation including IHC. Similar to the training set, the assay was not able to differentiate squamous cell carcinomas originating from different sources and falsely predicted them as lung.

The assay also made putative tissue of origin diagnoses for eight out of eleven samples which remained CUP after standard diagnostic tests. One of the CUP cases was especially interesting. A male patient with a history of prostate cancer was diagnosed with metastatic carcinoma in lung and pleura. Serum PSA tests and IHC with PSA antibodies on metastatic tissue were negative, so the pathologist's diagnosis was CUP with an inclination toward gastrointestinal tumors. The assay strongly (posterior probability 0.99) predicted the tissue of origin as colon.

Discussion. In this study, microarray-based expression profiling on primary tumors was used to identify candidate Markers for use with metastases. The fact that primary tumors can be used to discover tumor of origin Markers for metastases is consistent with several recent findings. For example, Weigelt and colleagues have shown that gene expression profiles of primary breast tumors are maintained in distant metastases. Weigelt et al. (2003). Backus and colleagues identified putative Markers for detecting breast cancer metastasis using a genome-wide gene expression analysis of breast and other tissues and demonstrated that mammaglobin and CK19 detected clinically actionable metastasis in breast sentinel lymph nodes with 90% sensitivity and 94% specificity. Backus et al. (2005).

During the development of the assay, selection was focused on six cancer types, including lung, pancreas and colon which are among the most prevalent in CUP (Ghosh et al. (2005); and Pavlidis et al. (2005)) and breast, ovarian and prostate for which treatment could be potentially most beneficial for patients. Ghosh et al. (2005). However, additional tissue types and Markers can be added to the panel as long as the overall accuracy of the assay is not compromised and, if applicable, the logistics of the RTPCR reactions are not encumbered.

The microarray-based studies with primary tissue confirmed the specificity and sensitivity of known Markers. As a result, the majority of tissue specific Markers have high specificity for the tissues studied here. A recent study found that, using IHC, PSCA is overexpressed in prostate cancer metastases. Lam et al. (2005). Dennis et al. (2002) also demonstrated that PSCA could be used as a tumor of origin Marker for pancreas and prostate. Strong expression of PSCA in some prostate tissues at the RNA level was present but, because due to inclusion of PSA in the assay, prostate and pancreatic cancers can now be segregated. A novel finding of this study was the use of F5 as a complementary (to PSCA) Marker for pancreatic tissue of origin. In both the microarray data set with primary tissue and the qRTPCR data set with FFPE metastases, F5 was found to complement PSCA.

Previous investigators have generated CUP assays using IHC (Brown et al. (1997); DeYoung et al. (2000); and Dennis et al. (2005a)) or microarrays. Su et al. (2001); Ramaswamy et al. (2001); and Bloom et al. (2004). More recently, SAGE has been coupled to a small qRTPCR Marker panel. Dennis et al. (2002); and Buckhaults et al. (2003). This study is the first to combine microarray-based expression profiling with a small panel of qRTPCR assays. The microarray studies with primary tissue identified some, but not all, of the same tissue of origin Markers as those identified previously by SAGE studies. This finding is not surprising given studies that have demonstrated that a modest agreement between SAGE- and DNA microarray-based profiling data exists and that the correlation improves for genes with higher expression levels. van Ruissen et al. (2005); and Kim et al. (2003). For example, Dennis and colleagues identified PSA, MG, PSCA, and HUMSPB while Buckhaults and coworkers (Buckhaults et al. (2003)) identified PDEF. Execution of the CUP assay is preferably by qRTPCR because it is a robust technology and may have performance advantages over IHC. Al-Mulla et al. (2005); and Haas et al. (2005). Further, as shown herein, the qRTPCR protocol has been improved through the use of gene-specific primers in a one-step reaction. This is the first demonstration of the use of gene-specific primers in a one-step qRTPCR reaction with FFPE tissue. Other investigators have either done a two-step qRTPCR (cDNA synthesis in one reaction followed by qPCR) or have used random hexamers or truncated gene-specific primers. Abrahamsen et al. (2003); Specht et al. (2001); Godfrey et al. (2000); Cronin et al. (2004); and Mikhitarian et al. (2004).

In summary, the 78% overall accuracy of the assay for six tissue types compares favorably to other studies. Brown et al. (1997); DeYoung et al. (2000); Dennis et al. (2005a); Su et al. (2001); Ramaswamy et al. (2001); and Bloom et al. (2004).

### EXAMPLE 3

In this study classifier using gene marker portfolios were built by choosing from MVO and using this classifier to predict tissue origin and cancer status for five major cancer types including breast, colon, lung, ovarian and prostate. Three hundred and seventy eight primary cancer, 23 benign proliferative epithelial lesions and 103 normal snap-frozen human tissue specimens were analyzed by using Affymetrix human U133A GeneChip. Leukocyte samples were also analyzed in order to subtract gene expression potentially masked by co-expression in leukocyte background cells. A novel MVO-based bioinformatics method was developed to select gene marker portfolios for tissue of origin and cancer status. The data demonstrated that a panel of 26 genes could be used as a classifier to accurately predict the tissue of origin and cancer status among the 5 cancer types. Thus a multi-cancer classification method is obtainable by determining gene expression profiles of a reasonably small number of gene markers.

Table 7 shows the Markers identified for the tissue origins indicated. For gene descriptions see Table 15.

TABLE 7

| Tissue | SEQ ID NO: | Name |
|---|---|---|
| Lung | 59 | SP-B |
| | 60 | TTF1 |
| | 61 | DSG3 |
| Pancreas | 66 | PSCA |
| | 67 | F5 |
| | 71 | ITGB6 |
| | 72 | TGM2 |
| | 84 | HNRPA0 |
| Colon | 85 | HPT1 |
| | 77 | FABP1 |
| | 78 | CDX1 |
| | 79 | GUCY2C |
| Prostate | 86 | PSA |
| | 80 | hKLK2 |
| Breast | 63 | MGB1 |
| | 81 | PIP |
| | 64 | PDEF |
| Ovarian | 82 | HE4 |
| | 83 | PAX8 |
| | 65 | WT1 |

The sample set included a total of 299 metastatic colon, breast, pancreas, ovary, prostate, lung and other carcinomas and primary prostate cancer samples. QC based on histological evaluation, RNA yield and expression of control gene beta-actin was implemented. Other samples category included metastasis originated from stomach (5), kidney (6), cholangio/gallbladder (4), liver (2), head and neck (4), ileum (1) carcinomas and one mesothelioma. Table 8 summarizes the results.

TABLE 8

| | | | RNA | ACTB |
|---|---|---|---|---|
| Tissue type | Collected | Histology QC | isolation QC | Cut-off QC |
| Lung | 41 | 37 | 36 | 25 |
| Pancreas | 63 | 57 | 49 | 41 |
| Colon | 45 | 42 | 42 | 31 |
| Breast | 40 | 35 | 35 | 34 |
| Ovarian | 37 | 36 | 35 | 33 |
| Prostate | 27 | 27 | 25 | 19 |
| Other | 46 | 34 | 29 | 23 |
| Total | 299 | 268 | 251 | 205 |

Testing the above samples resulted in the narrowing of the Marker set to those in Table 9 with the results seen in Table 10.

TABLE 9
Final Marker Table

| | | |
|---|---|---|
| Lung | surfactant-associated protein | SP-B |
| | thyroid transcription factor 1 | TTF1 |
| | desmoglein 3 | DSG3 |
| Pancreas | prostate stem cell antigen | PSCA |
| | coagulation factor 5 | F5 |
| Colon | intestinal peptide-associated transporter | HPT1 |
| Prostate | prostate-specific antigen | PSA |
| Breast | Mammaglobin | MGB |
| | Ets transcription factor | PDEF |
| Ovary | Wilms tumor 1 | WT1 |

TABLE 10

| Cancer | Samples # | Marker | Correct | Sensitivity % | Wrong | Specificity % |
|---|---|---|---|---|---|---|
| Lung | 25/180 | SP-B | 13/25 | 52 | 0/180 | 100 |
| | | TTF | 12/25 | 48 | 1/180 | 99 |
| | | DSG3 | 5/25 | 20 | 0/180 | 100 |
| Pancreas | 41/164 | PSCA | 24/41 | 59 | 6/164 | 96 |
| | | F5 | 6/41 | 15 | 4/164 | 98 |
| Colon | 31/174 | HPT1 | 22/31 | 71 | 2/174 | 99 |
| Breast | 33/172 | MGB | 23/33 | 70 | 3/172 | 98 |
| | | PDEF | 16/33 | 48 | 1/172 | 99 |
| Prostate | 19/186 | PSA | 19/19 | 100 | 0/186 | 100 |
| | | PDEF | 19/19 | 100 | 2/186 | 99 |
| Ovarian | 33/172 | WT1 | 24/33 | 71 | 1/172 | 99 |
| Total | 205 | | | | | |

The results showed that out of 205 paraffin embedded metastatic tumors; 166 samples (81%) had conclusive assay results, Table 11.

TABLE 11

| | | | | | Accuracy |
|---|---|---|---|---|---|
| | Candidate | Correct | Incorrect | No | (%) |
| Lung | SP-B + TFF + DSG3 | 19 | 0 | 6 | 76 |
| Pancreas | PSCA + F5 | 27 | 1 | 13 | 66 |
| Colon | HPT1 | 24 | 2 | 5 | 78 |
| Prostate | PSA | 19 | 0 | 0 | 100 |
| Breast | MGB + PDEF | 23 | 3 | 7 | 70 |
| Ovarian | WT1 | 23 | 2 | 8 | 70 |
| Other | | 20 | 3 | | 87 |
| Overall | | 155 | 11 | 39 | 76 |

Of the false positive results, many false derived from histologically and embryologically similar tissues, Table 12.

TABLE 12

| Sample ID | Diagnosis | Predicted |
|---|---|---|
| OV_26 | Ovarian | Breast |
| Br_24 | Breast | Colon |
| Br_37 | Breast | Colon |
| CRC_25 | Colon | Ovarian |
| Pn_59 | Pancreas | Colon |
| Cont_27 | Stomach | pancreas |
| Cont_34 | Stomach | Colon |
| Cont_35 | Stomach | Colon |
| Cont_43 | Bile duct | Pancreas |
| Cont_44 | Bile duct | Pancreas |
| Cong_25 | Liver | pancreas |

The following parameters were considered for the model development:

Separate markers on female and male sets and calculate CUP probability separately for male and female patients. The male set included: SP_B, TTF1, DSG3, PSCA, F5, PSA, HPT1; the female set included: SP_B, TTF1, DSG3, PSCA, F5, HPT1, MGB, PDEF, WT1. Background expression was excluded from the assay results: Lung: SP_B, TTF1, DSG3; Ovary: WT1; and Colon: HPT1.

The CUP model was adjusted to the CUP prevalence (%): lung 23, pancreas 16, colorectal 9, breast 3, ovarian 4, prostate 2, other 43. The prevalence for breast and ovarian adjusted to 0% for male patients, and prostate adjusted to 0% for female patients.

The following steps were taken:

Place markers on similar scale.

Reduce number of variables from 12 to 8 by selecting minimum value from each tissue specific set.

Leave out 1 sample. Build model from remaining samples. Test left out sample. Repeat until 100% of samples are tested.

Randomly leave out ˜50% of samples (˜50% per tissue). Build model from remaining samples. Test ˜50% of samples. Repeat for 3 different random splits.

Classification accuracy was adjusted to cancer types prevalence

To produce the results summarized in Table 13 with the raw data shown in Table 14

Although the foregoing invention has been described in some detail by way of illustration and example for purposes of clarity of understanding, the descriptions and examples should not be construed as limiting the scope of the invention.

TABLE 13

| | Breast | Colon | Lung | Other | Ovary | Pancreas | Prostate | Overall | Adjusted |
|---|---|---|---|---|---|---|---|---|---|
| Correct | 23 | 29 | 22 | 19 | 24 | 35 | 19 | 171 | |
| NoTest | 3 | 2 | 2 | | 2 | 3 | 0 | 12 | |
| Incorrect | 7 | 0 | 1 | 4 | 7 | 3 | 0 | 22 | |
| Prevalence | 0.03 | 0.09 | 0.23 | 0.43 | 0.04 | 0.16 | 0.02 | | |
| Tested/total % | 91 | 94 | 92 | 100 | 94 | 93 | 100 | 94 | 95 |
| Correct/total % | 70 | 94 | 88 | 83 | 73 | 85 | 100 | 89 | 89 |
| NoTest % | 9 | 6 | 8 | n/a | 6 | 7 | 0 | 6 | 5 |
| Correct | 23 | 25 | 19 | 20 | 20 | 24 | 19 | 150 | |
| NoTest % | 7 | 6 | 5 | | 10 | 15 | 0 | 43 | |
| Incorrect | 3 | 0 | 1 | 3 | 3 | 2 | 0 | 12 | |
| Prevalence | 0.03 | 0/09 | 0.23 | 0.43 | 0.04 | 0.16 | 0.02 | | |
| Tested/total % | 79 | 81 | 80 | 100 | 70 | 63 | 100 | 79 | 83 |
| Correct/total % | 70 | 81 | 76 | 87 | 61 | 59 | 100 | 73 | 76 |
| Correct/tested % | 88 | 100 | 95 | 87 | 87 | 92 | 100 | 93 | 91 |
| NoTest % | 21 | 19 | 20 | n/a | 30 | 37 | 0 | 21 | 17 |

TABLE 14

| Sam- | | | | | | | | | | | | | | | | | |
|---|---|---|---|---|---|---|---|---|---|---|---|---|---|---|---|---|---|
| ple | Gen- | Or- | | Pre- | BAC- | | | | | | | | | | | | |
| ID | der | igin | BK | diction | TIN | PBGD | Ave | CDH17 | DSG3 | F5 | HUMP | KLK3 | MG | PDEF | PSCA | TTF1 | WT1 |
| 128 | f | breast | lung | 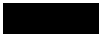 | 23.37 | 30.04 | 26.71 | 40.00 | 37.78 | 35.74 | 22.19 | 40.00 | 40.00 | 30.36 | 29.96 | 29.39 | 34.85 |
| 134 | f | breast | uk | breast | 19.60 | 27.00 | 23.30 | 40.00 | 31.27 | 30.83 | 40.00 | 40.00 | 29.51 | 25.07 | 24.67 | 40.00 | 34.13 |
| 166 | f | breast | uk | breast | 23.47 | 27.95 | 25.71 | 40.00 | 40.00 | 26.66 | 40.00 | 28.20 | 24.78 | 25.19 | 30.69 | 40.00 | 35.32 |
| 331 | f | breast | ovary | breast | 25.12 | 31.40 | 28.26 | 40.00 | 40.00 | 40.00 | 40.00 | 40.00 | 22.26 | 26.01 | 40.00 | 40.00 | 40.00 |
| 356 | f | breast | uk | breast | 28.59 | 33.89 | 31.24 | 40.00 | 34.01 | 40.00 | 40.00 | 40.00 | 35.73 | 33.19 | 30.72 | 40.00 | 40.00 |
| 163 | f | colon | uk | colon | 24.69 | 30.34 | 27.52 | 29.39 | 40.00 | 26.52 | 40.00 | 40.00 | 40.00 | 37.72 | 40.00 | 40.00 | 36.17 |
| 184 | m | colon | uk | colon | 22.47 | 28.63 | 25.55 | 26.22 | 33.26 | 28.76 | 40.00 | 40.00 | 40.00 | 34.07 | 33.44 | 40.00 | 31.64 |
| 339 | f | colon | uk | colon | 28.35 | 34.29 | 31.32 | 33.76 | 40.00 | 40.00 | 40.00 | 40.00 | 40.00 | 35.99 | 40.00 | 40.00 | 40.00 |
| 346 | m | colon | lung | colon | 23.15 | 28.77 | 25.96 | 26.36 | 40.00 | 32.64 | 20.89 | 40.00 | 40.00 | 32.47 | 40.00 | 26.75 | 30.58 |
| 363 | m | colon | uk | colon | 24.46 | 30.62 | 27.54 | 26.20 | 31.84 | 29.98 | 34.44 | 40.00 | 40.00 | 30.45 | 35.00 | 40.00 | 30.35 |
| 101 | m | lung | uk | lung | 24.68 | 28.79 | 26.74 | 40.00 | 40.00 | 39.34 | 21.57 | 40.00 | 40.00 | 28.21 | 27.47 | 40.00 | 35.76 |
| 106 | m | lung | uk | lung | 22.05 | 27.50 | 24.78 | 40.00 | 40.00 | 32.24 | 23.68 | 40.00 | 40.00 | 25.79 | 25.02 | 26.42 | 37.27 |
| 110 | m | lung | uk | lung | 29.19 | 32.32 | 30.76 | 40.00 | 40.00 | 40.00 | 21.21 | 40.00 | 40.00 | 32.77 | 32.43 | 30.70 | 36.13 |
| 112 | m | lung | uk | 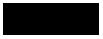 | 22.48 | 27.79 | 25.14 | 40.00 | 37.05 | 37.38 | 36.08 | 40.00 | 40.00 | 37.12 | 36.04 | 40.00 | 37.45 |
| 199 | f | lung | uk | lung | 21.21 | 27.07 | 24.14 | 35.65 | 25.56 | 31.23 | 40.00 | 40.00 | 28.94 | 32.19 | 27.95 | 32.14 | 31.60 |
| 200 | m | lung | uk | lung | 22.16 | 26.94 | 24.55 | 40.00 | 24.53 | 33.69 | 40.00 | 40.00 | 40.00 | 36.67 | 38.34 | 38.61 | 33.55 |
| 313323 | mm | lunglung | ukuk | 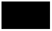 | 24.7623.82 | 30.0530.24 | 27.4127.03 | 38.4032.43 | 40.0031.82 | 40.0033.81 | 40.0040.00 | 40.0040.00 | 40.0040.00 | 40.0033.60 | 40.0028.12 | 40.0040.00 | 35.1131.87 |
| 325 | m | lung | uk | lung | 22.09 | 27.97 | 25.03 | 40.00 | 26.84 | 34.88 | 38.61 | 40.00 | 38.04 | 34.29 | 27.31 | 39.21 | 31.23 |
| 335 | m | lung | uk | 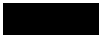 | 24.89 | 29.73 | 27.31 | 40.00 | 29.62 | 38.00 | 40.00 | 40.00 | 40.00 | 39.23 | 40.00 | 31.12 | 32.12 |
| 347 | m | lung | uk | lung | 23.40 | 29.08 | 26.24 | 40.00 | 26.72 | 37.21 | 40.00 | 40.00 | 40.00 | 36.10 | 30.76 | 40.00 | 39.44 |
| 374 | m | lung | uk | lung | 22.50 | 28.23 | 25.37 | 40.00 | 40.00 | 38.76 | 21.38 | 40.00 | 37.26 | 26.56 | 38.26 | 24.86 | 36.60 |
| 385 | f | lung | uk | lung | 21.65 | 26.44 | 24.05 | 37.05 | 40.00 | 34.51 | 19.89 | 40.00 | 40.00 | 27.36 | 40.00 | 23.72 | 37.09 |
| 114 | f | other | lung | other | 24.80 | 30.56 | 27.68 | 40.00 | 40.00 | 28.16 | 21.51 | 40.00 | 40.00 | 35.76 | 37.85 | 28.19 | 37.21 |
| 129 | m | other | lung | other | 21.49 | 28.25 | 24.87 | 39.47 | 40.00 | 28.86 | 20.65 | 40.00 | 40.00 | 32.98 | 40.00 | 28.14 | 31.11 |
| 179 | f | other | uk | other | 23.97 | 30.45 | 27.21 | 40.00 | 40.00 | 29.79 | 40.00 | 40.00 | 40.00 | 40.00 | 40.00 | 40.00 | 32.64 |
| 194 | m | other | uk | other | 25.28 | 32.47 | 28.88 | 40.00 | 40.00 | 28.90 | 40.00 | 40.00 | 40.00 | 40.00 | 40.00 | 34.75 | 35.41 |
| 302 | f | other | colon | 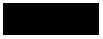 | 25.67 | 31.47 | 28.57 | 34.17 | 40.00 | 40.00 | 40.00 | 40.00 | 40.00 | 30.55 | 32.47 | 40.00 | 38.20 |
| 305 | m | other | uk | other | 23.80 | 29.74 | 26.77 | 29.64 | 40.00 | 34.06 | 40.00 | 40.00 | 40.00 | 31.82 | 40.00 | 40.00 | 40.00 |
| 317 | m | other | uk | 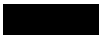 | 25.90 | 30.62 | 28.26 | 40.00 | 40.00 | 27.75 | 40.00 | 40.00 | 40.00 | 31.89 | 33.06 | 40.00 | 35.12 |
| 333 | f | other | uk | other | 22.45 | 28.82 | 25.64 | 30.54 | 40.00 | 37.01 | 40.00 | 40.00 | 40.00 | 37.85 | 40.00 | 40.00 | 40.00 |
| 334 | m | other | uk | other | 22.14 | 29.20 | 25.67 | 31.79 | 40.00 | 36.27 | 40.00 | 40.00 | 40.00 | 34.69 | 40.00 | 40.00 | 40.00 |
| 342 | f | other | uk | 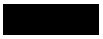 | 27.32 | 31.37 | 29.35 | 32.36 | 40.00 | 29.24 | 40.00 | 40.00 | 40.00 | 32.89 | 40.00 | 40.00 | 38.18 |
| 382 | m | other | uk | other | 25.04 | 30.22 | 27.63 | 40.00 | 40.00 | 36.13 | 40.00 | 40.00 | 40.00 | 38.30 | 40.00 | 40.00 | 34.91 |
| 404 | m | other | uk | other | 23.27 | 30.16 | 26.72 | 40.00 | 39.36 | 34.75 | 40.00 | 40.00 | 40.00 | 39.02 | 40.00 | 40.00 | 34.24 |
| 354 | f | ovary | uk | ovary | 24.62 | 31.54 | 28.08 | 40.00 | 40.00 | 34.90 | 40.00 | 40.00 | 40.00 | 36.62 | 40.00 | 40.00 | 29.71 |
| 148 | f | ovary | uk | 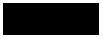 | 23.55 | 29.88 | 26.72 | 40.00 | 40.00 | 30.60 | 38.84 | 40.00 | 40.00 | 32.12 | 31.76 | 40.00 | 38.59 |
| 417 | f | pan | uk | pancre- | 23.42 | 29.46 | 26.44 | 28.28 | 38.96 | 29.05 | 37.01 | 40.00 | 40.00 | 30.15 | 30.23 | 40.00 | 30.69 |
| | | cre- | | as | | | | | | | | | | | | | |
| | | as | | | | | | | | | | | | | | | |
| 136 | m | pros- | lung | pros- | 22.37 | 26.95 | 24.66 | 40.00 | 40.00 | 29.47 | 23.69 | 21.38 | 40.00 | 24.70 | 24.28 | 30.89 | 31.16 |
| | | tate | | tate | | | | | | | | | | | | | |
| 407 | m | pros- | lung | pros- | 28.20 | 31.87 | 30.04 | 40.00 | 40.00 | 40.00 | 27.70 | 25.98 | 40.00 | 27.65 | 40.00 | 39.13 | 38.76 |
| | | tate | | tate | | | | | | | | | | | | | |
| 116 | f | CUP | uk | lung- | 21.66 | 27.31 | 24.49 | 28.95 | 27.86 | 31.06 | 40.00 | 40.00 | 30.28 | 33.49 | 29.31 | 40.00 | 38.11 |
| | | | | SCC | | | | | | | | | | | | | |
| 123 | m | CUP | lung | colon | 27.09 | 30.59 | 28.84 | 27.92 | 36.01 | 40.00 | 40.00 | 40.00 | 40.00 | 40.00 | 40.00 | 40.00 | 36.65 |
| 157 | m | CUP | uk | pancre- | 26.81 | 31.94 | 29.38 | 40.00 | 40.00 | 26.82 | 40.00 | 40.00 | 40.00 | 36.68 | 40.00 | 40.00 | 40.00 |
| | | | | as | | | | | | | | | | | | | |
| 177 | m | CUP | uk | pancre- | 25.44 | 31.52 | 28.48 | 40.00 | 40.00 | 27.15 | 40.00 | 40.00 | 40.00 | 39.67 | 40.00 | 40.00 | 34.71 |
| | | | | as | | | | | | | | | | | | | |
| 306 | m | CUP | uk | lung | 23.15 | 28.38 | 25.77 | 37.30 | 40.00 | 34.94 | 19.71 | 40.00 | 40.00 | 30.81 | 40.00 | 25.45 | 39.28 |
| 360 | m | CUP | uk | other | 21.14 | 27.43 | 24.29 | 33.97 | 36.98 | 32.72 | 40.00 | 40.00 | 40.00 | 27.75 | 40.00 | 40.00 | 40.00 |
| 372 | f | CUP | uk | ovary | 23.16 | 29.12 | 26.14 | 40.00 | 40.00 | 34.07 | 40.00 | 40.00 | 40.00 | 32.93 | 40.00 | 40.00 | 25.28 |
| 187 | f | CUP | uk | colon | 24.44 | 29.80 | 27.12 | 26.83 | 35.91 | 26.32 | 30.55 | 40.00 | 40.00 | 40.00 | 40.00 | 29.75 | 40.00 |

TABLE 15

| | SEQ ID | | |
|---|---|---|---|
| Name | NOs | Accession | Description |
| CDH17 | 62 | NM_004063 | Cadherin 17 |
| CDX1 | 78 | NM_001804 | Homeo box transcription factor 1 |
| DSG3 | 61/3 | NM_001944 | Desmoglein 3 |
| F5 | 67/6 | NM_000130 | Coagulation factor V |
| FABP1 | 71 | NM_001443 | Fatty acid binding protein 1, liver |
| GUCY2C | 79 | NM_004963 | Guanylate cyclase 2C |
| HE4 | 82 | NM_006103 | Putative ovarian carcinoma marker |
| KLK2 | 80 | BC005196 | Kallikrein 2, prostatic |
| HNRPA0 | 84 | NM_006805 | Heterogeneous nuclear |
| | | | ribonucleoprotein A0 |
| HPT1 | 85/4 | U07969 | Intestinal peptide-associated |
| | | | transporter |
| ITGB6 | 71 | NM_000888 | Integrin, beta 6 |
| KLK3 | 68 | NM_001648 | Kallikrein 3 |
| MGB1 | 63/7 | NM_002411 | Mammaglobin 1 |
| PAX8 | 83 | BC001060 | Paired box gene 8 |
| PBGD | 70 | NM_000190 | Hydroxymethylbilane synthase |
| PDEF | 64/8 | NM_012391 | Domain containing Ets |
| | | | transcription factor |
| PIP | 81 | NM_002652 | Prolactin-induced protein |
| PSA | 86/9 | U17040 | Prostate specific antigen precursor |
| PSCA | 66/5 | NM_005672 | Prostate stem cell antigen |
| SP-B | 59/1 | NM_198843 | Pulmonary surfactant-associated |
| | | | protein B |
| TGM2 | 72 | NM_004613 | Transglutaminase 2 |
| TTF1 | 60/2 | NM_003317 | Similar to thyroid transcription |
| | | | factor 1 |
| WT1 | 65/10 | NM_024426 | Wilms tumor 1 |
| β-actin | 69 | NM_001101 | β-actin |
| p73H | 87 | AB010153 | p53-related protein |
| KLK10 | 88 | NM_002776 | Kallikrein 10 |
| CLDN18 | 89 | NM_016369 | Claudin 18 |
| TR10 | 90 | BD280579 | Tumor necrosis factor receptor |
| SERPINA1 | 91 | NM_000295 | serpin peptidase inhibitor, clade A |
| | | | member 1 |
| KRT7 | 92 | NM_005556 | Keratin 7 |
| MMP11 | 93 | NM_005940 | matrix metallopeptidase 11 |
| | | | (stromelysin 3) |
| MUC4 | 94 | NM_018406 | Mucin 4 cell-surface associated |
| FLJ22041 | 95 | AK025694 | |
| BAX | 96 | NM_138763 | BCL2-assoc X protein transcript |
| | | | variant Δ |
| PITX1 | 97 | NM_002653 | paired-like homeodomain |
| | | | trans factor 1 |
| MGC: 10264 | 98 | BC005807 | stearoyl-CoA desaturase |
| | | | (Δ-9-desaturase) |

### REFERENCES

### US Patent Application Publications and Patents

| | | |
|---|---|---|
| 5242974 | 5545531 | 6218122 |
| 5384261 | 5554501 | 6339148 |
| 5405783 | 5556752 | 20020055627 |
| 5412087 | 5561071 | 20030194733 |
| 5424186 | 5571639 | 20030212264 |
| 5429807 | 5593839 | 20030232350 |
| 5436327 | 5599695 | 20030235820 |
| 5445934 | 5624711 | 20040005563 |
| 5472672 | 5658734 | 20040076955 |
| 5527681 | 5700637 | 20040219572 |
| 5529756 | 6004755 | 20050009067 |
| 5532128 | 6218114 | 20060029987 |

### Foreign Patent Publications and Patents

WO1998040403
WO2000055320
WO2004018999
WO2004031412
WO2004063355
WO2004077060
WO2005005601

### Journal Articles

### Abrahamsen et al. (2003) Towards quantitative mRNA analysis in paraffin-embedded tissues using real-time reverse transcriptase-polymerase chain reaction J Mol Diag 5:34-41

Al-Mulla et al. (2005) BRCA1 gene expression in breast cancer: a correlative study between real-time RT-PCR and immunohistochemistry J Histochem Cytochem 53:621-629
Argani et al. (2001) Discovery of new Markers of cancer through serial analysis of gene expression: prostate stem cell antigen is overexpressed in pancreatic adenocarcinoma Cancer Res 61:4320-4324

### Backus et al. (2005) Identification and characterization of optimal gene expression Markers for detection of breast cancer metastasis J Mol Diagn 7:327-336

### Bloom et al. (2004) Multi-platform, multi-site, microarray-based human tumor classification Am J Pathol 164:9-16

Borgono et al. (2004) Human tissue kallikreins: physiologic roles and applications in cancer Mol Cancer Res 2:257-280

### Brookes (1999) The essence of SNPs Gene 23:177-186

Brown et al. (1997) Immunohistochemical identification of tumor Markers in metastatic adenocarcinoma. A diagnostic adjunct in the determination of primary site Am J Clin Pathol 107:12-19

### Buckhaults et al. (2003) Identifying tumor origin using a gene expression-based classification map Cancer Res 63:4144-4149

### Cronin et al. (2004) Measurement of gene expression in archival paraffin-embedded tissue Am J Pathol 164:35-42

Cunha et al. (2006) Tissue-specificity of prostate specific antigens: Comparative analysis of transcript levels in prostate and non-prostatic tissues Cancer Lett 236:229-238

### Dennis et al. (2002) Identification from public data of molecular Markers of adenocarcinoma characteristic of the site of origin Can Res 62:5999-6005

Dennis et al. (2005a) Hunting the primary: novel strategies for defining the origin of tumors J Pathol 205:236-247
DeYoung et al. (2000) Immunohistologic evaluation of metastatic carcinomas of unknown origin: an algorithmic approach Semin Diagn Pathol 17:184-193

### Fleming et al. (2000) Mammaglobin, a breast-specific gene, and its utility as a Marker for breast cancer Ann NY Acad Sci 923:78-89

### Ghosh et al (2005) Management of patients with metastatic cancer of unknown primary Curr Probl Surg 42:12-66

### Godfrey et al. (2000) Quantitative mRNA expression analysis from formalin-fixed, paraffin-embedded tissues using 5′ nuclease quantitative reverse transcription-polymerase chain reaction J Mol Diag 2:84-91

### Haas et al. (2005) Combined application of RT-PCR and immunohistochemistry on paraffin embedded sentinel lymph nodes of prostate cancer patients Pathol Res Pract 200:763-770

Hwang et al. (2004) Wilms tumor gene product: sensitive and contextually specific Marker of serous carcinomas of ovarian surface epithelial origin Appl Immunohistochem Mol Morphol 12:122-126

### Ishikawa et al. (2005) Experimental trial for diagnosis of pancreatic ductal carcinoma based on gene expression profiles of pancreatic ductal cells Cancer Sci 96:387-393

Italiano et al. (2005) Epidermal growth factor receptor (EGFR) status in primary colorectal tumors correlates with EGFR expression in related metastatic sites: biological and clinical implications Ann Oncol 16:1503-1507
Jones et al. (2004) Comprehensive analysis of matrix metalloproteinase and tissue inhibitor expression in pancreatic cancer: increased expression of matrix metalloproteinase-7 predicts poor survival Clin Cancer Res 10:2832-2845

### Khoor et al. (1997) Expression of surfactant protein B precursor and surfactant protein B mRNA in adenocarcinoma of the lung Mod Pathol 10:62-67

### Kim (2003) Comparison of oligonucleotide-microarray and serial analysis of gene expression (SAGE) in transcript profiling analysis of megakaryocytes derived from CD34+ cells Exp Mol Med 35:460-466

### Lam et al. (2005) Prostate stem cell antigen is overexpressed in prostate cancer metastases Clin Can Res 11:2591-2596

### Lipshutz et al. (1999) High density synthetic oligonucleotide arrays Nature Genetics 21:S20-24

### Markowitz (1952) Portfolio Selection J Finance 7:77-91

McCarthy et al. (2003) Novel Markers of pancreatic adenocarcinoma in fine-needle aspiration: mesothelin and prostate stem cell antigen labeling increases accuracy in cytologically borderline cases Appl Immunohistochem Mol Morphol 11:238-243

### Mikhitarian et al. (2004) Enhanced detection of RNA from paraffin-embedded tissue using a panel of truncated gene-specific primers for reverse transcription BioTechniques 36:1-4

### Moniaux et al. (2004) Multiple roles of mucins in pancreatic cancer, a lethal and challenging malignancy Br J Cancer 91:1633-1638

### Nakamura et al. (2002) Expression of thyroid transcription factor-1 in normal and neoplastic lung tissues Mod Pathol 15:1058-1067

### Prasad et al. (2005) Gene expression profiles in pancreatic intraepithelial neoplasia reflect the effects of Hedgehog signaling on pancreatic ductal epithelial cells Cancer Res 65:1619-1626

### Ramaswamy et al. (2001) Multiclass cancer diagnosis using tumor gene expression signatures Proc Natl Acad Sci USA 98:15149-15154

### Specht et al. (2001) Quantitative gene expression analysis in microdissected archival formalin-fixed and paraffin-embedded tumor tissue Amer J Pathol 158:419-429

### Su et al. (2001) Molecular classification of human carcinomas by use of gene expression signatures Cancer Res 61:7388-7393

van Ruissen et al. (2005) Evaluation of the similarity of gene expression data estimated with SAGE and Affymetrix GeneChips BMC Genomics 6:91

### Weigelt et al. (2003) Gene expression profiles of primary breast tumors maintained in distant metastases Proc Natl Acad Sci USA 100:15901-15905

Lillemoe et al (2000) Pancreatic cancer: state-of-the-art care CA Cancer J Clin 50:241-68

### Warshau et al. (1992) N Engl J Med 326:4555-4565

### Kroep et al. (1999) Ann Oncol 10(Suppl 4):234-238

### Wiesenauer et al. (2003) Preoperative Predictors of Malignancy in Pancreatic Intraductal Papillary Mucinous Neoplasms Arch Surg 138:610-618

### Ros et al. (2001) Imaging features of pancreatic neoplasms JBR-BTR 84:239-49

### Ryu et al. (2002) Relationships and differentially expressed genes among pancreatic cancers examined by large-scale serial analysis of gene expression Cancer Res 62:819-26

### Ito et al. (2001) Molecular basis of T cell-mediated recognition of pancreatic cancer cells Cancer Res 61:2038-46

Gibson et al. (1978) Histological typing of tumors of the liver, biliary tract and pancreas WHO Geneva

1. A method of identifying pancreatic carcinoma comprising the steps of
a. obtaining a sample containing metastatic cells;
b. measuring Biomarkers associated with expression of F5, PSCA, ITGB6, KLK10, CLDN18, TR10 or FKBP10 Marker genes.
wherein the expression levels of the Marker genes above or below pre-determined cut-off levels are indicative of the presence of pancreatic cancer in the sample.

2. The method of claim 1 wherein the Marker genes are F5 and PSCA.

3. The method of claim 2 wherein the Marker genes further comprise or are replaced by ITGB6, KLK10, CLDN18, TR10 and/or FKBP10.

4. The method of one of claims 1-3 wherein gene expression is measured using at least one of SEQ ID NOs: 39-41 and 43-45.

5. A composition comprising at least one isolated sequence selected from SEQ ID NOs: 39-41 and 43-45.

6. A kit for conducting an assay according to one of claims 1-3 comprising: Biomarker detection reagents.

7. A microarray or gene chip for performing the method of one of claims 1-3.

8. A diagnostic/prognostic portfolio comprising isolated nucleic acid sequences, their complements, or portions thereof of a combination of genes according to one of claims 1-3, or 1-3 where the combination is sufficient to measure or characterize gene expression in a biological sample having metastatic cells relative to cells from different carcinomas or normal tissue.

9. A method according to one of claims 1-3, or 1-3 further comprising measuring expression of at least one gene constitutively expressed in the sample.


SEQUENCE LISTING (98 sequences)

SEQ ID NO: 1 - DNA, 476 bp, human
gaaaaaccagccactgctttacaggacagggggttgaagctgagccccgcctcacacccacccccatgcactcaaagattggattttacagctacttgcaattcaaaattcagaagaataaaaaatgggaacatacagaactctaaaagatagacatcagaaattgttaagttaagctttttcaaaaaatcagcaattccccagcgtagtcaagggtggacactgcacgctctggcatgatgggatggcgaccgggcaagctttcttcctcgagatgctctgctgcttgagagctattgctttgttaagatataaaaaggggtttctttttgtctttctgtaaggtggacttccagattttgattgaaagtcctagggtgattctatttctgctgtgatttatctgctgaaagctcagctggggttgtgcaagctagggacccattcctgtgtaatacaatgtctgcaccaatgct

SEQ ID NO: 2 - DNA, 493 bp, human
gtgattcaaatgggttttccacgctagggcggggcacagattggagagggctctgtgctgacatggctctggactctaaagaccaaacttcactctgggcacactctgccagcaaagaggactcgcttgtaaataccaggatttttttttttttttgaagggaggacgggagctggggagaggaaagagtcttcaacataacccacttgtcactgacacaaaggaagtgccccctccccggcaccctctggccgcctaggctcagcggcgaccgccctccgcgaaaatagtttgtttaatgtgaacttgtagctgtaaaacgctgtcaaaagttggactaaatgcctagtttttagtaatctgtacattttgttgtaaaaagaaaaaccactcccagtccccagcccttcacattttttatgggcattgacaaatctgtgtatattatttggcagtttggtatttgcggcgtcagtctttttctgttgtaact

SEQ ID NO: 3 - DNA, 545 bp, human
ccatcccatagaagtccagcagacaggatttgttaagtgccagactttgtcaggaagtcaaggagcttctgctttgtccgcctctgggtctgtccagccagctgtttccatccctgaccctctgcagcatggtaactatttagtaacggagacttactcggcttctggttccctcgtgcaaccttccactgcaggctttgatccacttctcacacaaaatgtgatagtgacagaaagggtgatctgtcccatttccagtgttcctggcaacctagctggcccaacgcagctacgagggtcacatactatgctctgtacagaggatccttgctcccgtctaatatgaccagaatgagctggaataccacactgaccaaatctggatctttggactaaagtattcaaaatagcatagcaaagctcactgtattgggctaataatttggcacttattagcttctctcataaactgatcacgattataaattaaatgtttgggttcataccccaaaagcaatatgttgtcactcctaattctcaagtac

SEQ ID NO: 4 - DNA, 284 bp, human
ctgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtc

SEQ ID NO: 5 - DNA, 394 bp, human
  misc_feature (58)..(58): n is a, c, g, or t
ttcctgaggcacatcctaacgcaagtttgaccatgtatgtttgcaccccttttccccnaaccctgaccttcccatgggccttttccaggattccnaccnggcagatcagttttagtganacanatccgcntgcagatggcccctccaaccntttntgttgntgtttccatggcccagcattttccacccttaaccctgtgttcaggcacttnttcccccaggaagccttccctgcccaccccatttatgaattgagccaggtttggtccgtggtgtcccccgcacccagcaggggacaggcaatcaggagggcccagtaaaggctgagatgaagtggactgagtagaactggaggacaagagttgacgtgagttcctgggagtttccagagatg

SEQ ID NO: 6 - DNA, 470 bp, human
  misc_feature (61)..(61): n is a, c, g, or t
atcctctacagccagatgtcacagggatacgtctactttcacttggtgctggagaattcanaagtcaagaacatgctaagcntaagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctggaaggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaag

SEQ ID NO: 7 - DNA, 396 bp, human
gcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaactttctgcaagacctttggctcacagaactgcagggtatggtgagaaaccaactacggattgctgcaaaccacaccttctctttcttatgtctttttact

SEQ ID NO: 8 - DNA, 491 bp, human
gagtggggcccttaaactggattcaaaaaatgctctaaacataggaatggttgaagaggtcttgcagtcttcagatgaaactaaatctctagaagaggcacaagaatggctaaagcaattcatccaagggccaccggaagtaattagagctttgaaaaaatctgtttgttcaggcagagagctatatttggaggaagcattacagaacgaaagagatcttttaggaacagtttggggtgggcctgcaaatttagaggctattgctaagaaaggaaaatttaataaataattggtttttcgtgtggatgtactccaagtaaagctccagtgactaatatgtataaatgttaaatgatattaaatatgaacatcagttaaaaaaaaaattctttaaggctactattaatatgcagacttacttttaatcatttgaaatctgaactcatttacctcatttcttgccaattactcccttgggtatttactgcgta

SEQ ID NO: 9 - DNA, 265 bp, human
tggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcc

SEQ ID NO: 10 - DNA, 441 bp, human
atagatgtacatacctccttgcacaaatggaggggaattcattttcatcactgggagtgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaatccattgtttaaagatggtcgtgtgtgtgtgtgtgtgtgtgtgtgtgttgtgttgtgttttgttttttaagggagggaatttattatttaccgttgcttgaaattactgtgtaaatatatgtctgataatgatttgctctttgacaactaaaattaggactgtataagtactagatgcatcactgggtgttgatcttacaagat

SEQ ID NO: 11 - DNA, 21 bp, human
cacagccccgacctttgatga

SEQ ID NO: 12 - DNA, 19 bp, human
ggtcccagagcccgtctca

SEQ ID NO: 13 - DNA, 26 bp, human
agctgtccagctgcaaaggaaaagcc

SEQ ID NO: 14 - DNA, 75 bp, human
cacagccccgacctttgatgagaactcagctgtccagctgcaaaggaaaagccaagtgagacgggctctgggacc

SEQ ID NO: 15 - DNA, 17 bp, human
ccaacccagacccgcgc

SEQ ID NO: 16 - DNA, 21 bp, human
cgcccatgccgctcatgttca

SEQ ID NO: 17 - DNA, 21 bp, human
cccgccatctcccgcttcatg

SEQ ID NO: 18 - DNA, 78 bp, human
ccaacccagacccgcgcttccccgccatctcccgcttcatgggcccggcgagcggcatgaacatgagcggcatgggcg

SEQ ID NO: 19 - DNA, 23 bp, human
gagagaaggagaagataactcaa

SEQ ID NO: 20 - DNA, 22 bp, human
actccagagattcggtaggtga

SEQ ID NO: 21 - DNA, 26 bp, human
attgccaagattacttcagattacca

SEQ ID NO: 22 - DNA, 97 bp, human
gcagagaaggagaagataactcaaaaagaaacccaattgccaagattacttcagattaccaagcaacccagaaaatcacctaccgaatctctggagt

SEQ ID NO: 23 - DNA, 21 bp, human
tccctcggcagtggaagctta

SEQ ID NO: 24 - DNA, 24 bp, human
tcctcaaactctgtgtgcctggta

SEQ ID NO: 25 - DNA, 29 bp, human
ccaaaatcaatggtactcatgcccgactg

SEQ ID NO: 26 - DNA, 95 bp, human
tccctcggcagtggaagcttacaaaacgactgggaagtttccaaaatcaatggtactcatgcccgactgtctaccaggcacacagagtttgagga

SEQ ID NO: 27 - DNA, 21 bp, human
agttgctgatggtcctcatgc

SEQ ID NO: 28 - DNA, 24 bp, human
cacttgtggattgattgtcttgga

SEQ ID NO: 29 - DNA, 23 bp, human
ccctctcccagcactgctacgca

SEQ ID NO: 30 - DNA, 107 bp, human
agttgctgatggtcctcatgctggcggccctctcccagcactgctacgcaggctctggctgccccttattggagaatgtgatttccaagacaatcaatccacaagtg

SEQ ID NO: 31 - DNA, 20 bp, human
cgcccacctggacatctgga

SEQ ID NO: 32 - DNA, 23 bp, human
cactggtcgaggcacagtagtga

SEQ ID NO: 33 - DNA, 25 bp, human
gtcagcggcctggatgaaagagcgg

SEQ ID NO: 34 - DNA, 86 bp, human
cgcccacctggacatctggaagtcagcggcctggatgaaagagcggacttcacctggggcgattcactactgtgcctcgaccagtg

SEQ ID NO: 35 - DNA, 23 bp, human
gcggagcccaatacagaatacac

SEQ ID NO: 36 - DNA, 19 bp, human
cggggctactccaggcaca

SEQ ID NO: 37 - DNA, 25 bp, human
tcagaggcattcaggatgtgcgacg

SEQ ID NO: 38 - DNA, 80 bp, human
gcggagcccaatacagaatacacacgcacggtgtcttcagaggcattcaggatgtgcgacgtgtgcctggagtagccccg

SEQ ID NO: 39 - DNA, 20 bp, human
ctgttgatggcaggcttggc

SEQ ID NO: 40 - DNA, 20 bp, human
ttgctcacctgggctttgca

SEQ ID NO: 41 - DNA, 21 bp, human
gcagccaggcactgccctgct

SEQ ID NO: 42 - DNA, 74 bp, human
ctgttgatggcaggcttggccctgcagccaggcactgccctgctgtgctactcctgcaaagcccaggtgagcaa

SEQ ID NO: 43 - DNA, 25 bp, human
tgaagaaatatcctgggattattca

SEQ ID NO: 44 - DNA, 27 bp, human
tatgtggtatcttctggaatatcatca

SEQ ID NO: 45 - DNA, 27 bp, human
acaaagggaaacagatattgaagactc

SEQ ID NO: 46 - DNA, 87 bp, human
tgaagaaatatcctgggattattcagaatttgtacaaagggaaacagatattgaagactctgatgatattccagaagataccacata

SEQ ID NO: 47 - DNA, 19 bp, human
cccccagtgggtcctcaca

SEQ ID NO: 48 - DNA, 22 bp, human
aggatgaaacaagctgtgccga

SEQ ID NO: 49 - DNA, 26 bp, human
caggaacaaaagcgtgatcttgctgg

SEQ ID NO: 50 - DNA, 82 bp, human
cccccagtgggtcctcacagctgcccactgcatcaggaacaaaagcgtgatcttgctgggtcggcacagcttgtttcatcct

SEQ ID NO: 51 - DNA, 19 bp, human
gccctgaggcactcttcca

SEQ ID NO: 52 - DNA, 22 bp, human
cggatgtccacgtcacacttca

SEQ ID NO: 53 - DNA, 25 bp, human
cttccttcctgggcatggagtcctg

SEQ ID NO: 54 - DNA, 100 bp, human
gccctgaggcactcttccagccttccttcctgggcatggagtcctgtggcatccacgaaactaccttcaactccatcatgaagtgtgacgtggacatccg

SEQ ID NO: 55 - DNA, 22 bp, human
ccacacacagcctactttccaa

SEQ ID NO: 56 - DNA, 21 bp, human
tacccacgcgaatcactctca

SEQ ID NO: 57 - DNA, 27 bp, human
aacggcaatgcggctgcaacggcggaa

SEQ ID NO: 58 - DNA, 103 bp, human
ccacacacagcctactttccaagcggagccatgtctggtaacggcaatgcggctgcaacggcggaagaaaacagcccaaagatgagagtgattcgcgtgggta

SEQ ID NO: 59 - DNA, 2724 bp, human
ggtgccatggctgagtcacacctgctgcagtggctgctgctgctgctgcccacgctctgtggcccaggcactgctgcctggaccacctcatccttggcctgtgcccagggccctgagttctggtgccaaagcctggagcaagcattgcagtgcagagccctagggcattgcctacaggaagtctggggacatgtgggagccgatgacctatgccaagagtgtgaggacatcgtccacatccttaacaagatggccaaggaggccattttccaggacacgatgaggaagttcctggagcaggagtgcaacgtcctccccttgaagctgctcatgccccagtgcaaccaagtgcttgacgactacttccccctggtcatcgactacttccagaaccagactgactcaaacggcatctgtatgcacctgggcctgtgcaaatcccggcagccagagccagagcaggagccagggatgtcagaccccctgcccaaacctctgcgggaccctctgccagaccctctgctggacaagctcgtcctccctgtgctgcccggggccctccaggcgaggcctgggcctcacacacaggatctctccgagcagcaattccccattcctctcccctattgctggctctgcagggctctgatcaagcggatccaagccatgattcccaagggtgcgctagctgtggcagtggcccaggtgtgccgcgtggtacctctggtggcgggcggcatctgccagtgcctggctgagcgctactccgtcatcctgctcgacacgctgctgggccgcatgctgccccagctggtctgccgcctcgtcctccggtgctccatggatgacagcgctggcccaaggtcgccgacaggagaatggctgccgcgagactctgagtgccacctctgcatgtccgtgaccacccaggccgggaacagcagcgagcaggccataccacaggcaatgctccaggcctgtgttggctcctggctggacagggaaaagtgcaagcaatttgtggagcagcacacgccccagctgctgaccctggtgcccaggggctgggatgcccacaccacctgccaggccctcggggtgtgtgggaccatgtccagccctctccagtgtatccacagccccgacctttgatgagaactcagctgtccagaaaaagacaccgtcctttaaagtgctgcagtatggccagacgtggtggctcacacctgcaatcccagcaccttaggaggccgaggcaggaggatccttgaggtcaggagttcgagaccagcctcgccaacatggtgaaaccccatttctactaaaaatacaaaaaattagccaagtgtggtggcatatgcctgtaatcccaactactcagaaggccgaggcaggagaattacttgaacgcaggagaatcactgcagcccaggaggcagaggttgcagtgagccgagattgcaccactgcactccagcctgggtgacagagcaagactccatctcagtaaataaataaataaataaaaagcgctgcagtagctgtggcctcaccctgaagtcagcgggcccaggcctacctcactctctcccttggcagagaagcagacgtccatagctcctctccctcacaagcgctcccagcctgccctccagctgctgctctcccctcccagtctctactcactgggatgaggttaggtcatgaggacaccaaaaacctaaaaataaacaaaaagccaaacaagccttagcttttcttaaagactgaaatgcctggaagtgtccctttatttataaaataacttttgtcatatttcttatacatgtttcttgtaagaaattcagaaactacagacaaagagagtggaaattacccactgtcaggcctctgagcccaagctaagccatcatatcccctgtgccctgcacgtatacacccagatggcctgaagcaactgaagatccacaaaagaagtgaaaatagccagttcctgccttaactgatgacattccaccattgtgatttgttcctgccccaccctaactgatcaattgaccttgtgacaatacaccttccccacccttgagaaggtgctttgtaatattctccccacccaccccacgcccgcacccccgcacccttaagaaggtattttgtaatattctctccgccattgagaatgtgctttgtaagatccaccccctgcccacaaaaaattgctcctaactccaccgcctatcccaaacctacaagaactaatgataatcccaccaccctttgctgactctttttggactcagcccacctgcacccaggtgattaaaaagctttattgttcacacaaagcctgtttggtagtctcttcacagggaagcatgtgacacccacaatcccacctagcccaggagagagctacggcagggtgtgtgttttgacactgagcttggggctttttccatcttctccccacagcctctggctccacacctccaccgttcaagcgccagaaagagctgtctatgcagcctgctcttgggcctggggatgagacacacaattcattggctcctggattttaagtagacatttgtaaatctatagctaactactgtccttaaagccattgtttccattacaaaatccaactctctgagagaaaagggtgttttaaatttaaaaaaataaaaacaaaaaagtttgattgagaaaaaaaaaaaaaaa

SEQ ID NO: 60 - DNA, 2352 bp, human
gaaacttaaaggtgtttaccttgtcatcagcatgtaagctaattatctcgggcaagatgtaggcttctattgtcttgttgctttagcgcttacgccccgcctctggtggctgcctaaaacctggcgccgggctaaaacaaacgcgaggcagcccccgagcctccactcaagccaattaaggaggactcggtccactccgttacgtgtacatccaacaagatcggcgttaaggtaacaccagaatatttggcaaagggagaaaaaaaaagcagcgaggcttcgccttccccctctcccttttttttcctcctcttccttcctcctccagccgccgccgaatcatgtcgatgagtccaaagcacacgactccgttctcagtgtctgacatcttgagtcccctggaggaaagctacaagaaagtgggcatggagggcggcggcctcggggctccgctggcggcgtacaggcagggccaggcggcaccgccaacagcggccatgcagcagcacgccgtggggcaccacggcgccgtcaccgccgcctaccacatgacggcggcgggggtgccccagctctcgcactccgccgtggggggctactgcaacggcaacctgggcaacatgagcgagctgccgccgtaccaggacaccatgaggaacagcgcctctggccccggatggtacggcgccaacccagacccgcgcttccccgccatctcccgcttcatgggcccggcgagcggcatgaacatgagcggcatgggcggcctgggctcgctgggggacgtgagcaagaacatggccccgctgccaagcgcgccgcgcaggaagcgccgggtgctcttctcgcaggcgcaggtgtacgagctggagcgacgcttcaagcaacagaagtacctgtcggcgccggagcgcgagcacctggccagcatgatccacctgacgcccacgcaggtcaagatctggttccagaaccaccgctacaaaatgaagcgccaggccaaggacaaggcggcgcagcagcaactgcagcaggacagcggcggcggcgggggcggcgggggcaccgggtgcccgcagcagcaacaggctcagcagcagtcgccgcgacgcgtggcggtgccggtcctggtgaaagacggcaaaccgtgccaggcgggtgcccccgcgccgggcgccgccagcctacaaggccacgcgcagcagcaggcgcagcaccaggcgcaggccgcgcaggcggcggcagcggccatctccgtgggcagcggtggcgccggccttggcgcacacccgggccaccagccaggcagcgcaggccagtctccggacctggcgcaccacgccgccagccccgcggcgctgcagggccaggtatccagcctgtcccacctgaactcctcgggctcggactacggcaccatgtcctgctccaccttgctatacggtcggacctggtgagaggacgccgggccggccctagcccagcgctctgcctcaccgcttccctcctgcccgccacacagaccaccatccaccgctgctccacgcgcttcgacttttcttaacaacctggccgcgtttagaccaaggaacaaaaaaaccacaaaggccaaactgctggacgtctttctttttttccccccctaaaatttgtgggtttttttttttaaaaaaagaaaatgaaaaacaaccaagcgcatccaatctcaaggaatctttaagcagagaagggcataaaacagctttggggtgtctttttttggtgattcaaatgggttttccacgctagggcggggcacagattggagagggctctgtgctgacatggctctggactctaaagaccaaacttcactctgggcacactctgccagcaaagaggactcgcttgtaaataccaggatttttttttttttttgaagggaggacgggagctggggagaggaaagagtcttcaacataacccacttgtcactgacacaaaggaagtgccccctccccggcaccctctggccgcctaggctcagcggcgaccgccctccgcgaaaatagtttgtttaatgtgaacttgtagctgtaaaacgctgtcaaaagttggactaaatgcctagtttttagtaatctgtacattttgttgtaaaaagaaaaaccactcccagtccccagcccttcacattttttatgggcattgacaaatctgtgtatattatttggcagtttggtatttgcggcgtcagtctttttctgttgtaacttatgtagatatttggcttaaatatagttcctaagaagcttctaataaattatacaaattaaaaagattctttttctgattaaaaaaaaaaaaaaaaaa

SEQ ID NO: 61 - DNA, 3336 bp, human
ttttcttagacattaactgcagacggctggcaggatagaagcagcggctcacttggactttttcaccagggaaatcagagacaatgatggggctcttccccagaactacaggggctctggccatcttcgtggtggtcatattggttcatggagaattgcgaatagagactaaaggtcaatatgatgaagaagagatgactatgcaacaagctaaaagaaggcaaaaacgtgaatgggtgaaatttgccaaaccctgcagagaaggagaagataactcaaaaagaaacccaattgccaagattacttcagattaccaagcaacccagaaaatcacctaccgaatctctggagtgggaatcgatcagccgccttttggaatctttgttgttgacaaaaacactggagatattaacataacagctatagtcgaccgggaggaaactccaagcttcctgatcacatgtcgggctctaaatgcccaaggactagatgtagagaaaccacttatactaacggttaaaattttggatattaatgataatcctccagtattttcacaacaaattttcatgggtgaaattgaagaaaatagtgcctcaaactcactggtgatgatactaaatgccacagatgcagatgaaccaaaccacttgaattctaaaattgccttcaaaattgtctctcaggaaccagcaggcacacccatgttcctcctaagcagaaacactggggaagtccgtactttgaccaattctcttgaccgagagcaagctagcagctatcgtctggttgtgagtggtgcagacaaagatggagaaggactatcaactcaatgtgaatgtaatattaaagtgaaagatgtcaacgataacttcccaatgtttagagactctcagtattcagcacgtattgaagaaaatattttaagttctgaattacttcgatttcaagtaacagatttggatgaagagtacacagataattggcttgcagtatatttctttacctctgggaatgaaggaaattggtttgaaatacaaactgatcctagaactaatgaaggcatcctgaaagtggtgaaggctctagattatgaacaactacaaagcgtgaaacttagtattgctgtcaaaaacaaagctgaatttcaccaatcagttatctctcgataccgagttcagtcaaccccagtcacaattcaggtaataaatgtaagagaaggaattgcattccgtcctgcttccaagacatttactgtgcaaaaaggcataagtagcaaaaaattggtggattatatcctgggaacatatcaagccatcgatgaggacactaacaaagctgcctcaaatgtcaaatatgtcatgggacgtaacgatggtggatacctaatgattgattcaaaaactgctgaaatcaaatttgtcaaaaatatgaaccgagattctactttcatagttaacaaaacaatcacagctgaggttctggccatagatgaatacacgggtaaaacttctacaggcacggtatatgttagagtacccgatttcaatgacaattgtccaacagctgtcctcgaaaaagatgcagtttgcagttcttcaccttccgtggttgtctccgctagaacactgaataatagatacactggcccctatacatttgcactggaagatcaacctgtaaagttgcctgccgtatggagtatcacaaccctcaatgctacctcggccctcctcagagcccaggaacagatacctcctggagtataccacatctccctggtacttacagacagtcagaacaatcggtgtgagatgccacgcagcttgacactggaagtctgtcagtgtgacaacaggggcatctgtggaacttcttacccaaccacaagccctgggaccaggtatggcaggccgcactcagggaggctggggcctgccgccatcggcctgctgctccttggtctcctgctgctgctgttggccccccttctgctgttgacctgtgactgtggggcaggttctactgggggagtgacaggtggttttatcccagttcctgatggctcagaaggaacaattcatcagtggggaattgaaggagcccatcctgaagacaaggaaatcacaaatatttgtgtgcctcctgtaacagccaatggagccgatttcatggaaagttctgaagtttgtacaaatacgtatgccagaggcacagcggtggaaggcacttcaggaatggaaatgaccactaagcttggagcagccactgaatctggaggtgctgcaggctttgcaacagggacagtgtcaggagctgcttcaggattcggagcagccactggagttggcatctgttcctcagggcagtctggaaccatgagaacaaggcattccactggaggaaccaataaggactacgctgatggggcgataagcatgaattttctggactcctacttttctcagaaagcatttgcctgtgcggaggaagacgatggccaggaagcaaatgactgcttgttgatctatgataatgaaggcgcagatgccactggttctcctgtgggctccgtgggttgttgcagttttattgctgatgacctggatgacagcttcttggactcacttggacccaaatttaaaaaacttgcagagataagccttggtgttgatggtgaaggcaaagaagttcagccaccctctaaagacagcggttatgggattgaatcctgtggccatcccatagaagtccagcagacaggatttgttaagtgccagactttgtcaggaagtcaaggagcttctgctttgtccgcctctgggtctgtccagccagctgtttccatccctgaccctctgcagcatggtaactatttagtaacggagacttactcggcttctggttccctcgtgcaaccttccactgcaggctttgatccacttctcacacaaaatgtgatagtgacagaaagggtgatctgtcccatttccagtgttcctggcaacctagctggcccaacgcagctacgagggtcacatactatgctctgtacagaggatccttgctcccgtctaatatgaccagaatgagctggaataccacactgaccaaatctggatctttggactaaagtattcaaaatagcatagcaaagctcactgtattgggctaataatttggcacttattagcttctctcataaactgatcacgattataaattaaatgtttgggttcataccccaaaagcaatatgttgtcactcctaattctcaagtactattcaaattgtagtaaatcttaaagtttttcaaaaccctaaaatcatattcgc

SEQ ID NO: 62 - DNA, 3697 bp, human
agggagtgttcccgggggagatactccagtcgtagcaagagtctcgaccactgaatggaagaaaaggacttttaaccaccattttgtgacttacagaaaggaatttgaataaagaaaactatgatacttcaggcccatcttcactccctgtgtcttcttatgctttatttggcaactggatatggccaagaggggaagtttagtggacccctgaaacccatgacattttctatttatgaaggccaagaaccgagtcaaattatattccagtttaaggccaatcctcctgctgtgacttttgaactaactggggagacagacaacatatttgtgatagaacgggagggacttctgtattacaacagagccttggacagggaaacaagatctactcacaatctccaggttgcagccctggacgctaatggaattatagtggagggtccagtccctatcaccatagaagtgaaggacatcaacgacaatcgacccacgtttctccagtcaaagtacgaaggctcagtaaggcagaactctcgcccaggaaagcccttcttgtatgtcaatgccacagacctggatgatccggccactcccaatggccagctttattaccagattgtcatccagcttcccatgatcaacaatgtcatgtactttcagatcaacaacaaaacgggagccatctctcttacccgagagggatctcaggaattgaatcctgctaagaatccttcctataatctggtgatctcagtgaaggacatgggaggccagagtgagaattccttcagtgataccacatctgtggatatcatagtgacagagaatatttggaaagcaccaaaacctgtggagatggtggaaaactcaactgatcctcaccccatcaaaatcactcaggtgcggtggaatgatcccggtgcacaatattccttagttgacaaagagaagctgccaagattcccattttcaattgaccaggaaggagatatttacgtgactcagcccttggaccgagaagaaaaggatgcatatgttttttatgcagttgcaaaggatgagtacggaaaaccactttcatatccgctggaaattcatgtaaaagttaaagatattaatgataatccacctacatgtccgtcaccagtaaccgtatttgaggtccaggagaatgaacgactgggtaacagtatcgggacccttactgcacatgacagggatgaagaaaatactgccaacagttttctaaactacaggattgtggagcaaactcccaaacttcccatggatggactcttcctaatccaaacctatgctggaatgttacagttagctaaacagtccttgaagaagcaagatactcctcagtacaacttaacgatagaggtgtctgacaaagatttcaagaccctttgttttgtgcaaatcaacgttattgatatcaatgatcagatccccatctttgaaaaatcagattatggaaacctgactcttgctgaagacacaaacattgggtccaccatcttaaccatccaggccactgatgctgatgagccatttactgggagttctaaaattctgtatcatatcataaagggagacagtgagggacgcctgggggttgacacagatccccataccaacaccggatatgtcataattaaaaagcctcttgattttgaaacagcagctgtttccaacattgtgttcaaagcagaaaatcctgagcctctagtgtttggtgtgaagtacaatgcaagttcttttgccaagttcacgcttattgtgacagatgtgaatgaagcacctcaattttcccaacacgtattccaagcgaaagtcagtgaggatgtagctataggcactaaagtgggcaatgtgactgccaaggatccagaaggtctggacataagctattcactgaggggagacacaagaggttggcttaaaattgaccacgtgactggtgagatctttagtgtggctccattggacagagaagccggaagtccatatcgggtacaagtggtggccacagaagtaggggggtcttccttgagctctgtgtcagagttccacctgatccttatggatgtgaatgacaaccctcccaggctagccaaggactacacgggcttgttcttctgccatcccctcagtgcacctggaagtctcattttcgaggctactgatgatgatcagcacttatttcggggtccccattttacattttccctcggcagtggaagcttacaaaacgactgggaagtttccaaaatcaatggtactcatgcccgactgtctaccaggcacacagagtttgaggagagggagtatgtcgtcttgatccgcatcaatgatgggggtcggccacccttggaaggcattgtttctttaccagttacattctgcagttgtgtggaaggaagttgtttccggccagcaggtcaccagactgggatacccactgtgggcatggcagttggtatactgctgaccacccttctggtgattggtataattttagcagttgtgtttatccgcataaagaaggataaaggcaaagataatgttgaaagtgctcaagcatctgaagtcaaacctctgagaagctgaatttgaaaaggaatgtttgaatttatatagcaagtgctatttcagcaacaaccatctcatcctattacttttcatctaacgtgcattataattttttaaacagatattccctcttgtcctttaatatttgctaaatatttcttttttgaggtggagtcttgctctgtcgcccaggctggagtacagtggtgtgatcccagctcactgcaacctccgcctcctgggttcacatgattctcctgcctcagcttcctaagtagctgggtttacaggcacccaccaccatgcccagctaatttttgtatttttaatagagacggggtttcgccatttggccaggctggtcttgaactcctgacgtcaagtgatctgcctgccttggtctcccaatacaggcatgaaccactgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtcagagaagaaaaacgtgaggactcaggaaaaataaataaataaaagaacagccttttcccttagtattaacagaaatgtttctgtgtcattaaccatctttaatcaatgtgacatgttgctctttggctgaaattcttcaacttggaaatgacacagacccacagaaggtgttcaaacacaacctactctgcaaaccttggtaaaggaaccagtcagctggccagatttcctcactacctgccatgcatacatgctgcgcatgttttcttcattcgtatgttagtaaagttttggttattatatatttaacatgtggaagaaaacaagacatgaaaagagtggtgacaaatcaagaataaacactggttgtagtcagttttgtttgttaa

SEQ ID NO: 63 - DNA, 503 bp, human
gacagcggcttccttgatccttgccacccgcgactgaacaccgacagcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaactttctgcaagacctttggctcacagaactgcagggtatggtgagaaaccaactacggattgctgcaaaccacaccttctctttcttatgtctttttactacaaactacaagacaattgttgaaacctgctatacatgtttattttaataaattgatggca

SEQ ID NO: 64 - DNA, 1894 bp, human
gtctgacttcctcccagcacattcctgcactctgccgtgtccacactgccccacagacccagtcctccaagcctgctgccagctccctgcaagcccctcaggttgggccttgccacggtgccagcaggcagccctgggctgggggtaggggactccctacaggcacgcagccctgagacctcagagggccaccccttgagggtggccaggcccccagtggccaacctgagtgctgcctctgccaccagccctgctggcccctggttccgctggccccccagatgcctggctgagacacgccagtggcctcagctgcccacacctcttcccggcccctgaagttggcactgcagcagacagctccctgggcaccaggcagctaacagacacagccgccagcccaaacagcagcggcatgggcagcgccagcccgggtctgagcagcgtatcccccagccacctcctgctgccccccgacacggtgtcgcggacaggcttggagaaggcggcagcgggggcagtgggtctcgagagacgggactggagtcccagtccacccgccacgcccgagcagggcctgtccgccttctacctctcctactttgacatgctgtaccctgaggacagcagctgggcagccaaggcccctggggccagcagtcgggaggagccacctgaggagcctgagcagtgcccggtcattgacagccaagccccagcgggcagcctggacttggtgcccggcgggctgaccttggaggagcactcgctggagcaggtgcagtccatggtggtgggcgaagtgctcaaggacatcgagacggcctgcaagctgctcaacatcaccgcagatcccatggactggagccccagcaatgtgcagaagtggctcctgtggacagagcaccaataccggctgccccccatgggcaaggccttccaggagctggcgggcaaggagctgtgcgccatgtcggaggagcagttccgccagcgctcgcccctgggtggggatgtgctgcacgcccacctggacatctggaagtcagcggcctggatgaaagagcggacttcacctggggcgattcactactgtgcctcgaccagtgaggagagctggaccgacagcgaggtggactcatcatgctccgggcagcccatccacctgtggcagttcctcaaggagttgctactcaagccccacagctatggccgcttcattaggtggctcaacaaggagaagggcatcttcaaaattgaggactcagcccaggtggcccggctgtggggcatccgcaagaaccgtcccgccatgaactacgacaagctgagccgctccatccgccagtattacaagaagggcatcatccggaagccagacatctcccagcgcctcgtctaccagttcgtgcaccccatctgagtgcctggcccagggcctgaaacccgccctcaggggcctctctcctgcctgccctgcctcagccaggccctgagatgggggaaaacgggcagtctgctctgctgctctgaccttccagagcccaaggtcagggaggggcaaccaactgccccagggggatatgggtcctctggggccttcgggaccatggggcaggggtgcttcctcctcaggcccagctgctcccctggaggacagagggagacagggctgctccccaacacctgcctctgaccccagcatttccagagcagagcctacagaagggcagtgactcgacaaaggccacaggcagtccaggcctctctctgctccatccccctgcctcccattctgcaccacacctggcatggtgcagggagacatctgcacccctgagttgggcagccaggagtgcccccgggaatggataataaagatactagagaactg

SEQ ID NO: 65 - DNA, 3029 bp, human
ccaggcagctggggtaaggagttcaaggcagcgcccacacccgggggctctccgcaacccgaccgcctgtccgctcccccacttcccgccctccctcccacctactcattcacccacccacccacccagagccgggacggcagcccaggcgcccgggccccgccgtctcctcgccgcgatcctggacttcctcttgctgcaggacccggcttccacgtgtgtcccggagccggcgtctcagcacacgctccgctccgggcctgggtgcctacagcagccagagcagcagggagtccgggacccgggcggcatctgggccaagttaggcgccgccgaggccagcgctgaacgtctccagggccggaggagccgcggggcgtccgggtctgagccgcagcaaatgggctccgacgtgcgggacctgaacgcgctgctgcccgccgtcccctccctgggtggcggcggcggctgtgccctgcctgtgagcggcgcggcgcagtgggcgccggtgctggactttgcgcccccgggcgcttcggcttacgggtcgttgggcggccccgcgccgccaccggctccgccgccacccccgccgccgccgcctcactccttcatcaaacaggagccgagctggggcggcgcggagccgcacgaggagcagtgcctgagcgccttcactgtccacttttccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggagttgctgctgggagctccagctcagtgaaatggacagaagggcagagcaaccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttctcgttcagaccagctcaaaagacaccaaaggagacatacaggtgtgaaaccattccagtgtaaaacttgtcagcgaaagttctcccggtccgaccacctgaagacccacaccaggactcatacaggtaaaacaagtgaaaagcccttcagctgtcggtggccaagttgtcagaaaaagtttgcccggtcagatgaattagtccgccatcacaacatgcatcagagaaacatgaccaaactccagctggcgctttgaggggtctccctcggggaccgttcagtgtcccaggcagcacagtgtgtgaactgctttcaagtctgactctccactcctcctcactaaaaaggaaacttcagttgatcttcttcatccaacttccaagacaagataccggtgcttctggaaactaccaggtgtgcctggaagagttggtctctgccctgcctacttttagttgactcacaggccctggagaagcagctaacaatgtctggttagttaaaagcccattgccatttggtgtggattttctactgtaagaagagccatagctgatcatgtccccctgacccttcccttctttttttatgctcgttttcgctggggatggaattattgtaccattttctatcatggaatatttataggccagggcatgtgtatgtgtctgctaatgtaaactttgtcatggtttccatttactaacagcaacagcaagaaataaatcagagagcaaggcatcgggggtgaatcttgtctaacattcccgaggtcagccaggctgctaacctggaaagcaggatgtagttctgccaggcaacttttaaagctcatgcatttcaagcagctgaagaaaaaatcagaactaaccagtacctctgtatagaaatctaaaagaattttaccattcagttaattcaatgtgaacactggcacactgctcttaagaaactatgaagatctgagatttttttgtgtatgtttttgactcttttgagtggtaatcatatgtgtctttatagatgtacatacctccttgcacaaatggaggggaattcattttcatcactgggagtgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaatccattgtttaaagatggtcgtgtgtgtgtgtgtgtgtgtgtgtgtgtgtgtgttgtgttgtgttttgttttttaagggagggaatttattatttaccgttgcttgaaattactgtgtaaatatatgtctgataatgatttgctctttgacaactaaaattaggactgtataagtactagatgcatcactgggtgttgatcttacaagatattgatgataacacttaaaattgtaacctgcatttttcactttgctctcaattaaagtctattcaaaag

SEQ ID NO: 66 - DNA, 1064 bp, human
tttgaggccatataaagtcacctgaggccctctccaccacagcccaccagtgaccatgaaggctgtgctgcttgccctgttgatggcaggcttggccctgcagccaggcactgccctgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagcggggcccatgccctgcagccggctgctgccatccttgcgctgctccctgcactcggcctgctgctctggggacccggccagctctaggctctggggggccccgctgcagcccacactgggtgtggtgccccaggcctctgtgccactcctcacacacccggcccagtgggagcctgtcctggttcctgaggcacatcctaacgcaagtctgaccatgtatgtctgcgcccctgtcccccaccctgaccctcccatggccctctccaggactcccacccggcagatcggctctattgacacagatccgcctgcagatggcccctccaaccctctctgctgctgtttccatggcccagcattctccacccttaaccctgtgctcaggcacctcttcccccaggaagccttccctgcccaccccatctatgacttgagccaggtctggtccgtggtgtcccccgcacccagcaggggacaggcactcaggagggcccggtaaaggctgagatgaagtggactgagtagaactggaggacaggagtcgacgtgagttcctgggagtctccagagatggggcctggaggcctggaggaaggggccaggcctcacattcgtggggctccctgaatggcagcctcagcacagcgtaggcccttaataaacacctgttggataagccaaaaaaaaaaaaaaaaaaaaaaaaaaaaaaaaaaaaaaaaaaaaa

SEQ ID NO: 67 - DNA, 6962 bp, human
gcaagaactgcaggggaggaggacgctgccacccacagcctctagagctcattgcagctgggacagcccggagtgtggttagcagctcggcaagcgctgcccaggtcctggggtggtggcagccagcgggagcaggaaaggaagcatgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaatcaggtcattccgaaactcatcattgaatcaggaagaagaagagttcaatcttactgccctagctctggagaatggcactgaattcgtttcttcaaacacagatataattgttggttcaaattattcttccccaagtaatattagtaagttcactgtcaataaccttgcagaacctcagaaagccccttctcaccaacaagccaccacagctggttccccactgagacacctcattggcaagaactcagttctcaattcttccacagcagagcattccagcccatattctgaagaccctatagaggatcctctacagccagatgtcacagggatacgtctactttcacttggtgctggagaattcaaaagtcaagaacatgctaagcataagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctggaaggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaagcagagtggccacccaaagtttcctagagttagacataaatctctacaagtaagacaggatggaggaaagagtagactgaagaaaagccagtttctcattaagacacgaaaaaagaaaaaagagaagcacacacaccatgctcctttatctccgaggacctttcaccctctaagaagtgaagcctacaacacattttcagaaagaagacttaagcattcgttggtgcttcataaatccaatgaaacatctcttcccacagacctcaatcagacattgccctctatggattttggctggatagcctcacttcctgaccataatcagaattcctcaaatgacactggtcaggcaagctgtcctccaggtctttatcagacagtgcccccagaggaacactatcaaacattccccattcaagaccctgatcaaatgcactctacttcagaccccagtcacagatcctcttctccagagctcagtgaaatgcttgagtatgaccgaagtcacaagtccttccccacagatataagtcaaatgtccccttcctcagaacatgaagtctggcagacagtcatctctccagacctcagccaggtgaccctctctccagaactcagccagacaaacctctctccagacctcagccacacgactctctctccagaactcattcagagaaacctttccccagccctcggtcagatgcccatttctccagacctcagccatacaaccctttctccagacctcagccatacaaccctttctttagacctcagccagacaaacctctctccagaactcagtcagacaaacctttctccagccctcggtcagatgcccctttctccagacctcagccatacaaccctttctctagacttcagccagacaaacctctctccagaactcagccatatgactctctctccagaactcagtcagacaaacctttccccagccctcggtcagatgcccatttctccagacctcagccatacaaccctttctctagacttcagccagacaaacctctctccagaactcagtcaaacaaacctttccccagccctcggtcagatgcccctttctccagaccccagccatacaaccctttctctagacctcagccagacaaacctctctccagaactcagtcagacaaacctttccccagacctcagtgagatgcccctctttgcagatctcagtcaaattccccttaccccagacctcgaccagatgacactttctccagaccttggtgagacagatctttccccaaactttggtcagatgtccctttccccagacctcagccaggtgactctctctccagacatcagtgacaccacccttctcccggatctcagccagatatcacctcctccagaccttgatcagatattctacccttctgaatctagtcagtcattgcttcttcaagaatttaatgagtcttttccttatccagaccttggtcagatgccatctccttcatctcctactctcaatgatacttttctatcaaaggaatttaatccactggttatagtgggcctcagtaaagatggtacagattacattgagatcattccaaaggaagaggtccagagcagtgaagatgactatgctgaaattgattatgtgccctatgatgacccctacaaaactgatgttaggacaaacatcaactcctccagagatcctgacaacattgcagcatggtacctccgcagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctatggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactagaattgaacattcaaaaacccctggaagagactctttaagacctcaaaccatttagaatgggcaatgtattttacgctgtgttaaatgttaacagttttccactatttctctttcttttctattagtgaataaaattttatac

SEQ ID NO: 68 - DNA, 1464 bp, human
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 69 - DNA, 1793 bp, human
cgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctta

SEQ ID NO: 70 - DNA, 1526 bp, human
ccggaagtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtgtaaaaaaaaaaaaaaaaa

SEQ ID NO: 71 - DNA, 2397 bp, human
gcaagaactgaaacgaatggggattgaactgctttgcctgttctttctatttctaggaaggaatgatcacgtacaaggtggctgtgccctgggaggtgcagaaacctgtgaagactgcctgcttattggacctcagtgtgcctggtgtgctcaggagaattttactcatccatctggagttggcgaaaggtgtgataccccagcaaaccttttagctaaaggatgtcaattaaacttcatcgaaaaccctgtctcccaagtagaaatacttaaaaataagcctctcagtgtaggcagacagaaaaatagttctgacattgttcagattgcgcctcaaagcttgatccttaagttgagaccaggtggtgcgcagactctgcaggtgcatgtccgccagactgaggactacccggtggatttgtattacctcatggacctctccgcctccatggatgacgacctcaacacaataaaggagctgggctcccggctttccaaagagatgtctaaattaaccagcaactttagactgggcttcggatcttttgtggaaaaacctgtatcccctttcgtgaaaacaacaccagaagaaattgccaacccttgcagtagtattccatacttctgtttacctacatttggattcaagcacattttgccattgacaaatgatgctgaaagattcaatgaaattgtgaagaatcagaaaatttctgctaatattgacacacccgaaggtggatttgatgcaattatgcaagctgctgtgtgtaaggaaaaaattggctggcggaatgactccctccacctcctggtctttgtgagtgatgctgattctcattttggaatggacagcaaactagcaggcatcgtcattcctaatgacgggctctgtcacttggacagcaagaatgaatactccatgtcaactgtcttggaatatccaacaattggacaactcattgataaactggtacaaaacaacgtgttattgatcttcgctgtaacccaagaacaagttcatttatatgagaattacgcaaaacttattcctggagctacagtaggtctacttcagaaggactccggaaacattctccagctgatcatctcagcttatgaagaactgcggtctgaggtggaactggaagtattaggagacactgaaggactcaacttgtcatttacagccatctgtaacaacggtaccctcttccaacaccaaaagaaatgctctcacatgaaagtgggagacacagcttccttcagcgtgactgtgaatatcccacactgcgagagaagaagcaggcacattatcataaagcctgtggggctgggggatgccctggaattacttgtcagcccagaatgcaactgcgactgtcagaaagaagtggaagtgaacagctccaaatgtcaccacgggaacggctctttccagtgtggggtgtgtgcctgccaccctggccacatggggcctcgctgtgagtgtggcgaggacatgctgagcacagattcctgcaaggaggccccagatcatccctcctgcagcggaaggggtgactgctactgtgggcagtgtatctgccacttgtctccctatggaaacatttatgggccttattgccagtgtgacaatttctcctgcgtgagacacaaagggctgctctgcggaggtaacggcgactgtgactgtggtgaatgtgtgtgcaggagcggctggactggcgagtactgcaactgcaccaccagcacggactcctgcgtctctgaagatggagtgctctgcagcgggcgcggggactgtgtttgtggcaagtgtgtttgcacaaaccctggagcctcaggaccaacctgtgaacgatgtcctacctgtggtgacccctgtaactctaaacggagctgcattgagtgccacctgtcagcagctggccaagcccgagaagaatgtgtggacaagtgcaaactagctggtgcgaccatcagtgaagaagaagatttctcaaaggatggttctgtttcctgctctctgcaaggagaaaatgaatgtcttattacattcctaataactacagataatgaggggaaaaccatcattcacagcatcaatgaaaaagattgtccgaagcctccaaacattcccatgatcatgttaggggtttccctggctattcttctcatcggggttgtcctactgtgcatctggaagctactggtgtcatttcatgatcgtaaagaagttgccaaatttgaagcagaacgatcaaaagccaagtggcaaacgggaaccaatccactctacagaggatccacaagtacttttaaaaatgtaacttataaacacagggaaaaacaaaaggtagacctttccacagattgctagaactactttatgca

SEQ ID NO: 72 - DNA, 2118 bp, human
tggggagcccaagcagaaacgcaagctggtggctgaggtgtccctgcagaacccgctccctgtggccctggaaggctgcaccttcactgtggagggggccggcctgactgaggagcagaagacggtggagatcccagaccccgtggaggcaggggaggaagttaaggtgagaatggacctgctgccgctccacatgggcctccacaagctggtggtgaacttcgagagcgacaagctgaaggctgtgaagggcttccggaatgtcatcattggccccgcctaagggacccctgctcccagcctgctgagagcccccaccttgatcccaatccttatcccaagctagtgagcaaaatatgccccttcttgggccccagaccccagggcagggtgggcagcctatgggggctctcggaaatggaatgtgcccctggcccatctcagcctcctgagcctgtgggtccccactcaccccctttgctgtgaggaatgctctgtgccagaaacagtgggagccctgaccttggctgactggggctggggtgagagaggaaagacctacattccctctcctgcccagatgccctttggaaagccattgaccacccaccatattgtttgatctacttcatagctccttggagcaggcaaaaaagggacagcatgccccttggctggatcagggaatccagctccctagactgcatcccgtacctcttcccatgactgcacccagctccaggggcccttgggacagccagagctgggtggggacagtgataggcccaaggtcccctccacatcccagcagcccaagcttaatagccctccccctcaacctcaccattgtgaagcacctactatgtgctgggtgcctcccacacttgctggggctcacggggcctccaacccatttaatcaccatgggaaactgttgtgggcgctgcttccaggataaggagactgaggcttagagagaggaggcagccccctccacaccagtggcctcgtggttattagcaaggctgggtaatgtgaaggcccaagagcagagtctgggcctctgactctgagtccactgctccatttataaccccagcctgacctgagactgtcggagaggctgtctggggcctttatcaaaaaaagactcagccaagacaaggaggtagagaggggactgggggactgggagtcagagccctggctgggttcaggtcccacgtctggccaggcactgccttctcctctctgggcctttgtttccttgttggtcagaggagtgattgaaccagctcatctccaaggatcctctccactccatgtttgcaatgcttttatatggcccagccttgtaaataaccacaaggtccactccctgctccacgaagccttaagccataggcccaggatatttctgagagtgaaaccatgactgtgaccaccttctgtccccagccctgtcctggttccttcctatgcccaggtaccacccttcagaccccagttctaggggagaagagccctggacacccctgctctacccatgagcctgcccgctgcaatgcctagacttcccaacagccttagctgccagtgctggtcactaaccaacaaggttggcaccccagctaccccttctttgcagggctaaggcccccaaacatagcccctgccccggaggaagcttggggaacccatgagttgtcagctttgactttatctcctgctctttctacatgactgggcctcccttgggctggaagaattggggattctctattggaggtgagatcacagcctccagggccccccaaatcccagggaaggacttggagagaatcatgctgttgcatttagaactttctgctttgcacaggaaagagtcacacaattaatcaacatgtatattttctctatacatagagctctatttctctacggttttataaaagccttgggttccaaccaggcagtagatgtgcttctgaaccgcaaggagcaaacactgaaataaaatagtttatttttcacactcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 73 - DNA, 2832 bp, human
aaagctcaaaccgacaccctcacgcagatgatgacatcaactcttttttcttccccaagtgtacacaatgtgatggagactgttacgcaggagacagctcctccagatgaaatgaccacatcatttccctccagtgtcaccaacacactcatgatgacatcaaagactataacaatgacaacctccacagactccactcttggaaacacagaagagacatcaacagcaggaactgaaagttctaccccagtgacctcagcagtctcaataacagctggacaggaaggacaatcacgaacaacttcctggaggacctctatccaagacacatcagcttcttctcagaaccactggactcggagcacgcagaccaccagggaatctcaaaccagcaccctaacacacagaaccacttcaactccttctttctctccaagtgtacacaatgtgacagggactgtttctcagaagacatctccttcaggtgaaacagctacctcatccctctgtagtgtcacaaacacatccatgatgacatcagagaagataacagtgacaacctccacaggctccactcttggaaacccaggggagacatcatcagtacctgttactggaagtcttatgccagtcacctcagcagccttagtaacagttgatccagaaggacaatcaccagcaactttctcaaggacttctactcaggacacaacagctttttctaagaaccaccagactcagagcgtggagaccaccagagtatctcaaatcaacaccctcaacaccctcacaccggttacaacatcaactgttttatcctcaccaagtggattcaacccaagtggaacagtttctcaggagacattcccttctggtgaaacaaccatctcatccccttccagtgtcagcaatacattcctggtaacatcaaaggtgttcagaatgccaatctccagagactctactcttggaaacacagaggagacatcactatctgtaagtggaaccatttctgcaatcacttccaaagtttcaaccatatggtggtcagacactctgtcaacagcactctcccccagttctctacctccaaaaatatccacagctttccacacccagcagagtgaaggtgcagagaccacaggacggcctcatgagaggagctcattctctccaggtgtgtctcaagaaatatttactctacatgaaacaacaacatggccttcctcattctccagcaaaggccacacaacttggtcacaaacagaactgccctcaacatcaacaggtgctgccactaggcttgtcacaggaaatccatctacagggacagctggcactattccaagggtcccctctaaggtctcagcaataggggaaccaggagagcccaccacatactcctcccacagcacaactctcccaaaaacaacaggggcaggcgcccagacacaatggacacaagaaacggggaccactggagaggctcttctcagcagcccaagctacagtgtgactcagatgataaaaacggccacatccccatcttcttcacctatgctggatagacacacatcacaacaaattacaacggcaccatcaacaaatcattcaacaatacattccacaagcacctctcctcaggaatcaccagctgtttcccaaaggggtcacactcaagccccgcagaccacacaagaatcacaaaccacgaggtccgtctcccccatgactgacaccaagacagtcaccaccccaggttcttccttcacagccagtgggcactcgccctcagaaattgttcctcaggacgcacccaccataagtgcagcaacaacctttgccccagctcccaccggggatggtcacacaacccaggccccgaccacagcactgcaggcagcacccagcagccatgatgccaccctggggccctcaggaggcacgtcactttccaaaacaggtgcccttactctggccaactctgtagtgtcaacaccagggggcccagaaggacaatggacatcagcctctgccagcacctcacctgacacagcagcagccatgacccatacccaccaggctgagagcacagaggcctctggacaaacacagaccagcgaaccggcctcctcagggtcacgaaccacctcagcgggcacagctaccccttcctcatccggggcgagtggcacaacaccttcaggaagcgaaggaatatccacctcaggagagacgacaaggttttcatcaaacccctccagggacagtcacacaacccagtcaacaaccgaattgctgtccgcctcagccagtcatggtgccatcccagtaagcacaggaatggcgtcttcgatcgtccccggcacctttcatcccaccctctctgaggcctccactgcagggagaccgacaggacagtcaagcccaacttctcccagtgcctctcctcaggagacagccgccatttcccggatggcccagactcagaggacaagaaccagcagagggtctgacactatcagcctggcgtcccaggcaaccgacaccttctcaacagtcccacccacacctccatcgatcacatccactgggcttacatctccacaaacccagacccacactctgtcaccttcagggtctggtaaaaccttcaccacggccctcatcagcaacgccacccctcttcctgtcacctacgcttcctcggcatccacaggtcacaccacccctcttcatgtcaccgatgcttcctcagtatccacaggtcacgccacccctcttcctgtcaccagcccttcctcagtatccacaggtcacaccacccctctt

SEQ ID NO: 74 - DNA, 1607 bp, human
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcaaaa

SEQ ID NO: 75 - DNA, 1753 bp, human
cagccccgcccctacctgtggaagcccagccgcccgctcccgcggataaaaggcgcggagtgtccccgaggtcagcgagtgcgcgctcctcctcgcccgccgctaggtccatcccggcccagccaccatgtccatccacttcagctccccggtattcacctcgcgctcagccgccttctcgggccgcggcgcccaggtgcgcctgagctccgctcgccccggcggccttggcagcagcagcctctacggcctcggcgcctcacggccgcgcgtggccgtgcgctctgcctatgggggcccggtgggcgccggcatccgcgaggtcaccattaaccagagcctgctggccccgctgcggctggacgccgacccctccctccagcgggtgcgccaggaggagagcgagcagatcaagaccctcaacaacaagtttgcctccttcatcgacaaggtgcggtttctggagcagcagaacaagctgctggagaccaagtggacgctgctgcaggagcagaagtcggccaagagcagccgcctcccagacatctttgaggcccagattgctggccttcggggtcagcttgaggcactgcaggtggatgggggccgcctggaggcggagctgcggagcatgcaggatgtggtggaggacttcaagaataagtacgaagatgaaattaaccaccgcacagctgctgagaatgagtttgtggtgctgaagaaggatgtggatgctgcctacatgagcaaggtggagctggaggccaaggtggatgccctgaatgatgagatcaacttcctcaggaccctcaatgagacggagttgacagagctgcagtcccagatctccgacacatctgtggtgctgtccatggacaacagtcgctccctggacctggacggcatcatcgctgaggtcaaggcgcagtatgaggagatggccaaatgcagccgggctgaggctgaagcctggtaccagaccaagtttgagaccctccaggcccaggctgggaagcatggggacgacctccggaatacccggaatgagatttcagagatgaaccgggccatccagaggctgcaggctgagatcgacaacatcaagaaccagcgtgccaagttggaggccgccattgccgaggctgaggagcgtggggagctggcgctcaaggatgctcgtgccaagcaggaggagctggaagccgccctgcagcggggcaagcaggatatggcacggcagctgcgtgagtaccaggaactcatgagcgtgaagctggccctggacatcgagatcgccacctaccgcaagctgctggagggcgaggagagccggttggctggagatggagtgggagccgtgaatatctctgtgatgaattccactggtggcagtagcagtggcggtggcattgggctgaccctcgggggaaccatgggcagcaatgccctgagcttctccagcagtgcgggtcctgggctcctgaaggcttattccatccggaccgcatccgccagtcgcaggagtgcccgcgactgagccgcctcccaccactccactcctccagccaccacccacaatcacaagaagattcccacccctgcctcccatgcctggtcccaagacagtgagacagtctggaaagtgatgtcagaatagcttccaataaagcagcctcattctgaggcctgagtgatccacgtgaaaaaaaaaaaaaaaaaaaaaaaaaaaaaaaaaaaaaaaaaaaaaaaaaaa

SEQ ID NO: 76 - DNA, 2255 bp, human
gatggctccggccgcctggctccgcagcgcggccgcgcgcgccctcctgcccccgatgctgctgctgctgctccagccgccgccgctgctggcccgggctctgccgccggacgcccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttctcccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctgccaacactttcctctgaccatggcttggatgccctcaggggtgctgacccctgccaggccacgaatatcaggctagagacccatggccatctttgtggctgtgggcaccaggcatgggactgagcccatgtctcctcagggggatggggtggggtacaaccaccatgacaactgccgggagggccacgcaggtcgtggtcacctgccagcgactgtctcagactgggcagggaggctttggcatgacttaagaggaagggcagtcttgggcccgctatgcaggtcctggcaaacctggctgccctgtctccatccctgtccctcagggtagcaccatggcaggactgggggaactggagtgtccttgctgtatccctgttgtgaggttccttccaggggctggcactgaagcaagggtgctggggccccatggccttcagccctggctgagcaactgggctgtagggcagggccacttcctgaggtcaggtcttggtaggtgcctgcatctgtctgccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactgctatcctccaaagccattgtaaatgtgtgtacagtgtgtataaaccttcttcttctttttttttttttaaactgaggattgtcattaaacacagttgttttctaaaaaaaaaaaaaaaa

SEQ ID NO: 77 - DNA, 462 bp, human
agctctattgccaccatgagtttctccggcaagtaccaactgcagagccaggaaaactttgaagccttcatgaaggcaatcggtctgccggaagagctcatccagaaggggaaggatatcaagggggtgtcggaaatcgtgcagaatgggaagcacttcaagttcaccatcaccgctgggtccaaagtgatccaaaacgaattcacggtgggggaggaatgtgagctggagacaatgacaggggagaaagtcaagacagtggttcagttggaaggtgacaataaactggtgacaactttcaaaaacatcaagtctgtgaccgaactcaacggcgacataatcaccaataccatgacattgggtgacattgtcttcaagagaatcagcaagagaatttaaacaagtctgcatttcatattattttagtgtgtaaaattaatgtaataaagtgaactttgtttt

SEQ ID NO: 78 - DNA, 2108 bp, human
gggaccgcctcggaggcagaagagccgcgaggagccagcggagcaccgcgggctggggcgcagccacccgccgctcctcgagtcccctcgcccctttcccttcgtgccccccggcagcctccagcgtcggtccccaggcagcatggtgaggtctgctcccggaccctcgccaccatgtacgtgagctacctcctggacaaggacgtgagcatgtaccctagctccgtgcgccactctggcggcctcaacctggcgccgcagaacttcgtcagccccccgcagtacccggactacggcggttaccacgtggcggccgcagctgcagcggcagcgaacttggacagcgcgcagtccccggggccatcctggccggcagcgtatggcgccccactccgggaggactggaatggctacgcgcccggaggcgccgcggccgccgccaacgccgtggctcacggcctcaacggtggctccccggccgcagccatgggctacagcagccccgcagactaccatccgcaccaccacccgcatcaccacccgcaccacccggccgccgcgccttcctgcgcttctgggctgctgcaaacgctcaaccccggccctcctgggcccgccgccaccgctgccgccgagcagctgtctcccggcggccagcggcggaacctgtgcgagtggatgcggaagccggcgcagcagtccctcggcagccaagtgaaaaccaggacgaaagacaaatatcgagtggtgtacacggaccaccagcggctggagctggagaaggagtttcactacagtcgctacatcaccatccggaggaaagccgagctagccgccacgctggggctctctgagaggcaggttaaaatctggtttcagaaccgcagagcaaaggagaggaaaatcaacaagaagaagttgcagcagcaacagcagcagcagccaccacagccgcctccgccgccaccacagcctccccagcctcagccaggtcctctgagaagtgtcccagagcccttgagtccggtgtcttccctgcaagcctcagtgtctggctctgtccctggggttctggggccaactgggggggtgctaaaccccaccgtcacccagtgacccaccggggtctgcagcggcagagcaattccaggctgagccatgaggagcgtggactctgctagactcctcaggagagacccctcccctcccacccacagccatagacctacagacctggctctcagaggaaaaatgggagccaggagtaagacaagtgggatttggggcctcaagaaatatactctcccagatttttactttttcccatctggctttttctgccactgaggagacagaaagcctccgctgggcttcattccggactggcagaagcattgcctggactgaccacaccaaccaggccttcatcctcctccccagctcttctcttcctagatctgcaggctacacctctggctagagccgaggggagagagggactcaagggaaaggcaagcttgaggccaagatggctgctgcctgctcatggccctcggaggtccagctgggcctcctgcctccgggcaggcaaggtttacactgcggaagccaaaggcagctaagatagaaagctggactgaccaaagactgcagaacccccaggtggcctgcgtcttttttctcttcccttcccagaccaggaaaggcttggctggtgtatgcacagggtgtggtatgagggggtggttattggactccaggcctgaccagggggcccgaacagggacttgtttagagagcctgtcaccagagcttctctgggctgaatgtatgtcagtgctataaatgccagagccaacctggacttcctgtcattttcacaatcttggggctgatgaagaagggggtggggggagtttgtgttgttgttgctgctgtttgggttgttggtctgtgtaacatccaagccagagtttttaaagccttctggatccatggggggagaagtgatatggtgaagggaagtggggagtatttgaacacagttgaattttttctaaaaagaaaaagagataaatgagctttccagaaaaaaaaaaaaaaaaaaa

SEQ ID NO: 79 - DNA, 3745 bp, human
cgcaaagcaagtgggcacaaggagtatggttctaacgtgattggggtcatgaagacgttgctgttggacttggctttgtggtcactgctcttccagcccgggtggctgtcctttagttcccaggtgagtcagaactgccacaatggcagctatgaaatcagcgtcctgatgatgggcaactcagcctttgcagagcccctgaaaaacttggaagatgcggtgaatgaggggctggaaatagtgagaggacgtctgcaaaatgctggcctaaatgtgactgtgaacgctactttcatgtattcggatggtctgattcataactcaggcgactgccggagtagcacctgtgaaggcctcgacctactcaggaaaatttcaaatgcacaacggatgggctgtgtcctcatagggccctcatgtacatactccaccttccagatgtaccttgacacagaattgagctaccccatgatctcagctggaagttttggattgtcatgtgactataaagaaaccttaaccaggctgatgtctccagctagaaagttgatgtacttcttggttaacttttggaaaaccaacgatctgcccttcaaaacttattcctggagcacttcgtatgtttacaagaatggtacagaaactgaggactgtttctggtaccttaatgctctggaggctagcgtttcctatttctcccacgaactcggctttaaggtggtgttaagacaagataaggagtttcaggatatcttaatggaccacaacaggaaaagcaatgtgattattatgtgtggtggtccagagttcctctacaagctgaagggtgaccgagcagtggctgaagacattgtcattattctagtggatcttttcaatgaccagtacttggaggacaatgtcacagcccctgactatatgaaaaatgtccttgttctgacgctgtctcctgggaattcccttctaaatagctctttctccaggaatctatcaccaacaaaacgagactttgctcttgcctatttgaatggaatcctgctctttggacatatgctgaagatatttcttgaaaatggagaaaatattaccacccccaaatttgctcatgctttcaggaatctcacttttgaagggtatgacggtccagtgaccttggatgactggggggatgttgacagtaccatggtgcttctgtatacctctgtggacaccaagaaatacaaggttcttttgacctatgatacccacgtaaataagacctatcctgtggatatgagccccacattcacttggaagaactctaaacttcctaatgatattacaggccggggccctcagatcctgatgattgcagtcttcaccctcactggagctgtggtgctgctcctgctcgtcgctctcctgatgctcagaaaatatagaaaagattatgaacttcgtcagaaaaaatggtcccacattcctcctgaaaatatctttcctctggagaccaatgagaccaatcatgttagcctcaagatcgatgatgacaaaagacgagatacaatccagagactacgacagtgcaaatacgacaaaaagcgagtgattctcaaagatctcaagcacaatgatggtaatttcactgaaaaacagaagatagaattgaacaagttgcttcagattgactattacaacctgaccaagttctacggcacagtgaaacttgataccatgatcttcggggtgatagaatactgtgagagaggatccctccgggaagttttaaatgacacaatttcctaccctgatggcacattcatggattgggagtttaagatctctgtcttgtatgacattgctaagggaatgtcatatctgcactccagtaagacagaagtccatggtcgtctgaaatctaccaactgcgtagtggacagtagaatggtggtgaagatcactgattttggctgcaattccattttacctccaaaaaaggacctgtggacagctccagagcacctccgccaagccaacatctctcagaaaggagatgtgtacagctatgggatcatcgcacaggagatcattctgcggaaagaaaccttctacactttgagctgtcgggaccggaatgagaagattttcagagtggaaaattccaatggaatgaaacccttccgcccagatttattcttggaaacagcagaggaaaaagagctagaagtgtacctacttgtaaaaaactgttgggaggaagatccagaaaagagaccagatttcaaaaaaattgagactacacttgccaagatatttggactttttcatgaccaaaaaaatgaaagctatatggataccttgatccgacgtctacagctatattctcgaaacctggaacatctggtagaggaaaggacacagctgtacaaggcagagagggacagggctgacagacttaactttatgttgcttccaaggctagtggtaaagtctctgaaggagaaaggctttgtggagccggaactatatgaggaagttacaatctacttcagtgacattgtaggtttcactactatctgcaaatacagcacccccatggaagtggtggacatgcttaatgacatctataagagttttgaccacattgttgatcatcatgatgtctacaaggtggaaaccatcggtgatgcgtacatggtggctagtggtttgcctaagagaaatggcaatcggcatgcaatagacattgccaagatggccttggaaatcctcagcttcatggggacctttgagctggagcatcttcctggcctcccaatatggattcgcattggagttcactctggtccctgtgctgctggagttgtgggaatcaagatgcctcgttattgtctatttggagatacggtcaacacagcctctaggatggaatccactggcctccctttgagaattcacgtgagtggctccaccatagccatcctgaagagaactgagtgccagttcctttatgaagtgagaggagaaacatacttaaagggaagaggaaatgagactacctactggctgactgggatgaaggaccagaaattcaacctgccaacccctcctactgtggagaatcaacagcgtttgcaagcagaattttcagacatgattgccaactctttacagaaaagacaggcagcagggataagaagccaaaaacccagacgggtagccagctataaaaaaggcactctggaatacttgcagctgaataccacagacaaggagagcacctatttttaaacctaaatgaggtataaggactcacacaaattaaaatacagctgcactgaggcagcgacctcaagtgtcctgaaagcttacattttcctgagacctcaatgaagcagaaatgtacttaggcttggctgccctgtctggaacatggactttcttgcatgaatcagatgtgtgttctcagtgaaataactaccttccactctggaaccttattccagcagttgttccagggagcttctacctggaaaagaaaagaaatgaatagactatctagaacttgagaagattttattcttatttcatttattttttgtttgtttatttttatcgtttttgtttactggctttccttctgtattcataagattttttaaattgtcataattatattttaaatacccatcttcattaaagtatatttaactcataatttttgcagaaaatatgctatatattaggcaagaataaaagctaaagg

SEQ ID NO: 80 - DNA, 901 bp, human
agccccaaactcaccacctggccgtggacacctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgcttcgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtgggggtgattctgggggtccacttgtctgtaatggtgtgcttcaaggtatcacatcatggggccctgagccatgtgccctgcctgaaaagcctgctgtgtacaccaaggtggtgcattaccggaagtggatcaaggacaccatcgcagccaacccctgagtgcccctgtcccacccctacctctagtaaatttaagtccacctcaaaaaaaaaaaaaaaaaaaaaaaaaaaaa

SEQ ID NO: 81 - DNA, 618 bp, human
ggggaccacttctctgggacacattgccttctgttttctccagcatgcgcttgctccagctcctgttcagggccagccctgccaccctgctcctggttctctgcctgcagttgggggccaacaaagctcaggacaacactcggaagatcataataaagaattttgacattcccaagtcagtacgtccaaatgacgaagtcactgcagtgcttgcagttcaaacagaattgaaagaatgcatggtggttaaaacttacctcattagcagcatccctctacaaggtgcatttaactataagtatactgcctgcctatgtgacgacaatccaaaaaccttctactgggacttttacaccaacagaactgtgcaaattgcagccgtcgttgatgttattcgggaattaggcatctgccctgatgatgctgctgtaatccccatcaaaaacaaccggttttatactattgaaatcctaaaggtagaataatggaagccctgtctgtttgccacacccaggtgatttcctctaaagaaacttggctggaatttctgctgtggtctataaaataaacttcttaacatgcttctacaaaaaaaaaaaaaaaaaaaaaaaaaaaaaa

SEQ ID NO: 82 - DNA, 594 bp, human
gtcggtttaggactttctgcctccactattgctatcggtactggaatagcaggcatttcaacatctgtcacgaccttccatagcctatataatgacttatctgctagcatcacagacatatcacaaactttatcagtcctccaggcccaagttgaatctttagctgcagttgtcctccaaaaccgccgaggccttgacttacttactgcttaaagaggaggactctgcatattcttaaatgaggagtgttgtttttacataaatcaatctggcctggtgtatgacaacataaaaaaattcaaggatagagcccaaaaacttaccaaccaagcaagtaatttcactgaacccccttgggcactccctaattgggtgtcctgggtcctcccaattcttagtcctttaatacccatttttctcctccttttattcagaccttgtatcttctgtttagcttctcaattcatccaaaaccatatccaggccatcaccaatcattctatacgacaaatgtttcttataacatccccacaatatcaccccttaccacaagacctcccttcaacttaatctctcccgatataggttcccaca

SEQ ID NO: 83 - DNA, 1372 bp, human
gaattcggcgatgcctcacaactccatcagatctggccatggagggctgaaccagctgggaggggcctttgtgaatggcagacctctgccggaagtggtccgccagcgcatcgtagacctggcccaccagggtgtaaggccctgcgacatctctcgccagctccgcgtcagccatggttgcgtcagcaagatccttggcaggtactacgagactggcagcatccggcctggagtgatagggggctccaagcccaaggtggccacccccaaggtggtggagaagattggggactacaaacgccagaaccctaccatgtttgcctgggagatccgagaccggctcctggctgagggcgtctgtgacaatgacactgtgcccagtgtcagctccattaatagaatcatccggaccaaagtgcagcaaccattcaacctccctatggacagctgcgtggccaccaagtccctgagtcccggacacacgctgatccccagctcagctgtaactcccccggagtcaccccagtcggattccctgggctccacctactccatcaatgggctcctgggcatcgctcagcctggcagcgacaagaggaaaatggatgacagtgatcaggatagctgccgactaagcattgactcacagagcagcagcagcggaccccgaaagcaccttcgcacggatgccttcagccagcaccacctcgagccgctcgagtgcccatttgagcggcagcactacccagaggcctatgcctcccccagccacaccaaaggcgagcagggcctctacccgctgcccttgctcaacagcaccctggacgacgggaaggccaccctgaccccttccaacacgccactggggcgcaacctctcgactcaccagacctaccccgtggtggcagatcctcactcacccttcgccataaagcaggaaacccccgaggtgtccagttctagctccaccccttcctctttatctagctccgcctttttggatctgcagcaagtcggctccggggtcccgcccttcaatgcctttccccatgctgcctccgtgtacgggcagttcacgggccaggccctcctctcagggcgagagatggtggggcccacgctgcccggatacccaccccacatccccaccagcggacagggcagctatgcctcctctgccatcgcaggcatggtggcaggaagtgaatactctggcaatgcctatggccacaccccctactcctcctacagcgaggcctggcgcttccccaactccagcttgctgagttccccatattattacagttccacatcaaggccgagtgcaccgcccaccactgccacggcctttgaccatctgtagttgaagctt

SEQ ID NO: 84 - DNA, 2983 bp, human
gcccagataggggagcggaggtggcggcggcggcggtagcggtggccttggttgtcttccagtctcctcggctcgccctttagccggcaccgctccccttccctcccccttcctctcttccttccttccctccccttccctttttcccttccccgtcggtgagcggcgggggtggctccagcaacggctgggcccaagctgtgtagaggccttaaccaacgataacggcggcgacggcgaaacctcggagctcgcagggcgggggcaaggcccgggccttggagatggagaattctcagttgtgtaagctgttcatcggcggcctcaatgtgcagacgagtgagtcgggcctgcgcggccactttgaggcctttgggactctgacggactgcgtggtggtggtgaatccccagaccaagcgctcccgttgctttggcttcgtgacctactccaatgtggaggaggcggacgccgccatggccgcctcgccccatgccgtggacggcaacactgtggagctgaagcgggcggtgtcccgggaggattcggcgcggcccggtgcccacgccaaggttaagaagctctttgtcggaggccttaaaggagacgtggctgagggcgacctgatcgagcacttctcgcagtttggcaccgtggaaaaggccgagattattgccgacaagcagtccggcaagaagcgtggattcggcttcgtgtatttccagaatcacgacgcggcagacaaggccgcggtggtcaagttccatccgattcagggccatcgcgtggaggtgaagaaagcagtccccaaggaggatatctactccggtgggggtggaggcggctcccgatcctcccggggcggccgaggcggccgggggcgcggcggtggtcgagaccagaacggcctttccaagggcggcggcggcggttacaacagctacggtggttacggcggcggcggaggcggcggctacaatgcctacggaggcggcggcggcggttcgtcctacggtgggagcgactacggtaacggcttcggcggcttcggcagctacagccagcatcagtcctcctatgggcccatgaagagcggcggcggcggcggcggtggaggcagtagctggggcggtcgcagtaatagtggaccttacagaggcggctatggcggtgggggtggctatggaggcagctccttctaaaagaaaatttaaaatgcctgggagtggctataggggtagctctttccaacagcccaagtggggtcaactcctaagccccaccccctcacacacaccgccttccctgttttgcccttgggggagccacttctaaggctgcttacccttgggggtgttcctctatttgcctgccacctctcttgtctctccctctgaagatggactcggccccacatacacatttttgtgttacagtcattgatggactctatttttttattattacttggaccttggtcgtttttatactagcaaaatgtcttgttttaatttgtgttttttggggggagggagggagtgaacttgctgattctgtagcaaaacctgggtgggggttggggtggggggtagtttactttgttgtaaggacttgataacctggctacagcgttttctatgaaatctacttggatcccatgcctgaaatttggaagcatatgtacaaaaatcatttttacgttttatttttaataaatcattgtgtttgaccgtacatgtctaacattttttttctaggatccattccgtaccgttttttaagggatatttgtttaagactttacgtgttaattctttattcttgatgtgtacttagagaaacttaagaggtcctgtggtttttttcccctctcctgttgccctgctagttgcgtgttgaattatatcccttacaggcaaaacttttgaagtggtggatgtggctttttaaactcttaagtttctgtgcatccatctcttgtactaagcgaattgtttatcatcttgacatggttggtcatttctatgacaatttacttcaaactgtgtactgtgtagttctatatagtttgtgttaagcatgtcattcatataaactgtttaaaatttttcagatggcctagtttcatccctcttactggtttgtctgtaatgaatggttaaaaataagggttatattttaccctcaaatgcgtttttgtactttcagagcaggtttaaacgttttttttttttttttcctatatccgaactgttggcctcatggaaatccctttcccgatctttgtagcaccatctactggcagaatggcagagtagctgcgaaacaatttgtttaaaaacttgcttaagacaattgcatcagatttggaagttttgccatcaaaattctttgcagaattggaagttaacacatttgcttgtaactgagatgggcttcacaggaatgtagttgccagttcatatcacaatagccctttctatatgaggtttgaaaatgtaaactgctatgcatagcttgggcaatagccctaaattgctatgacaactaatgaaccagctacgtatactggtattttaggtgcaagttgtaaagcaaaatatctgtgtattctgcttggttaacaaatgtatatttgtagccctttcctgcaatagcattcaagttgttgtttataagagaagaacaaaagtgataataggtgaaaattgcctttctggatagaaatagagaatagcaacgtttatggatatcacaaataaagaattcaattctttacatgattgagtgagagtatgtataacctggtgggtgggttcagagtaccttttaatctagtatgcttaacttgatgttaatatttaacttaaatatttgacttacatgttgacgttgaaggctcaaagctatactaagaagctttctgaaagattgggctttaaaataaaataatattttaatattgaaaaaaaaaaaaaaaaaa

SEQ ID NO: 85 - DNA, 3345 bp, human
gaattccgtctcgaccactgaatggaagaaaaggacttttaaccaccattttgtgacttacagaaaggaatttgaataaagaaaactatgatacttcaggcccatcttcactccctgtgtcttcttatgctttatttggcaactggatatggccaagaggggaagtttagtggacccctgaaacccatgacattttctatttatgaaggccaagaaccgagtcaaattatattccagtttaaggccaatcctcctgctgtgacttttgaactaactggggagacagacaacatatttgtgatagaacgggagggacttctgtattacaacagagccttggacagggaaacaagatctactcacaatctccaggttgcagccctggacgctaatggaattatagtggagggtccagtccctatcaccatagaagtgaaggacatcaacgacaatcgacccacgtttctccagtcaaagtacgaaggctcagtaaggcagaactctcgcccaggaaagcccttcttgtatgtcaatgccacagacctggatgatccggccactcccaatggccagctttattaccagattgtcatccagcttcccatgatcaacaatgtcatgtactttcagatcaacaacaaaacgggagccatctctcttacccgagagggatctcaggaattgaatcctgctaagaatccttcctataatctggtgatctcagtgaaggacatgggaggccagagtgagaattccttcagtgataccacatctgtggatatcatagtgacagagaatatttggaaagcaccaaaacctgtggagatggtggaaaactcaactgatcctcaccccatcaaaatcactcaggtgcggtggaatgatcccggtgcacaatattccttagttgacaaagagaagctgccaagattcccattttcaattgaccaggaaggagatatttacgtgactcagcccttggaccgagaagaaaaggatgcatatgttttttatgcagttgcaaaggatgagtacggaaaaccactttcatatccgctggaaattcatgtaaaagttaaagatattaatgataatccacctacatgtccgtcaccagtaaccgtatttgaggtccaggagaatgaacgactgggtaacagtatcgggacccttactgcacatgacagggatgaagaaaatactgccaacagttttctaaactacaggattgtggagcaaactcccaaacttcccatggatggactcttcctaatccaaacctatgctggaatgttacagttagctaaacagtccttgaagaagcaagatactcctcagtacaacttaacgatagaggtgtctgacaaagatttcaagaccctttgttttgtgcaaatcaacgttattgatatcaatgatcagatccccatctttgaaaaatcagattatggaaacctgactcttgctgaagacacaaacattgggtccaccatcttaaccatccaggccactgatgctgatgagccatttactgggagttctaaaattctgtatcatatcataaagggagacagtgagggacgcctgggggttgacacagatccccataccaacaccggatatgtcataattaaaaagcctcttgattttgaaacagcagctgtttccaacattgtgttcaaagcagaaaatcctgagcctctagtgtttggtgtgaagtacaatgcaagttcttttgccaagttcacgcttattgtgacagatgtgaatgaagcacctcaattttcccaacacgtattccaagcgaaagtcagtgaggatgtagctataggcactaaagtgggcaatgtgactgccaaggatccagaaggtctggacataagctattcactgaggggagacacaagaggttggcttaaaattgaccacgtgactggtgagatctttagtgtggctccattggacagagaagccggaagtccatatcgggtacaagtggtggccacagaagtaggggggtcttccttaagctctgtgtcagagttccacctgatccttatggatgtgaatgacaaccctcccaggctagccaaggactacacgggcttgttcttctgccatcccctcagtgcacctggaagtctcattttcgaggctactgatgatgatcagcacttatttcggggtccccattttacattttccctcggcagtggaagcttacaaaacgactgggaagtttccaaaatcaatggtactcatgcccgactgtctaccaggcacacagactttgaggagagggcgtatgtcgtcttgatccgcatcaatgatgggggtcggccacccttggaaggcattgtttctttaccagttacattctgcagttgtgtggaaggaagttgtttccggccagcaggtcaccagactgggatacccactgtgggcatggcagttggtatactgctgaccacccttctggtgattggtataattttagcagttgtgtttatccgcataaagaaggataaaggcaaagataatgttgaaagtgctcaagcatctgaagtcaaacctctgagaagctgaatttgaaaaggaatgtttgaatttatatagcaagtgctatttcagcaacaaccatctcatcctattacttttcatctaacgtgcattataattttttaaacagatattccctcttgtcctttaatatttgctaaatatttcttttttgaggtggagtcttgctctgtcgcccaggctggagtacagtggtgtgatcccagctcactgcaacctccgcctcctgggttcacatgattctcctgcctcagcttcctaagtagctgggtttacaggcacccaccaccatgcccagctaatttttgtatttttaatagagacggggtttcgccatttggccaggctggtcttgaactcctgacgtcaagtgatctgcctgccttggtctcccaatacaggcatgaaccactgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtcagagaagaaaaaagtgaggactcaggaaaaataaataaataaaagaacagccttttgcggccgcgaattc

SEQ ID NO: 86 - DNA, 990 bp, human
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttctta

SEQ ID NO: 87 - DNA, 2820 bp, human
tggcaaaatcctggagccagaagaaaggacagcagcattgatcaatcttacagctaacatgttgtacctggaaaacaatgcccagactcaatttagtgagccacagtacacgaacctggggctcctgaacagcatggaccagcagattcggaacggctcctcgtccaccagtccctataacacagaccacgcgcagaacagcgtcacggcgccctcgccctacgcacagcccagccccaccttcgatgctctctctccatcacccgccatcccctccaacaccgactacccaggcccgcacagttccgacgtgtccttccagcagtcgagcaccgccaagtcggccacctggacgtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgagttcaacgagggacagattgcccctcctagtcatttgattcgagtagaggggaacagccatgcccagtatgtagaagatcccatcacaggaagacagagtgtgctggtaccttatgagccaccccaggttggcactgaattcacgacagtcttgtacaatttcatgtgtaacagcagttgtgttggagggatgaaccgccgtccaattttaatcattgttactctggaaaccagagatgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgcccgtttcgtcagaacacacatggtatccagatgacatccatcaagaaacgaagatccccagatgatgaactgttatacttaccagtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagtgagcctcaccatgtgagctcttcctatccctctcctaactgccagccccctaaaagcactcctgcttaatcttcaaagccttctccctagctcctccccttcctcttgtctgatttcttaggggaaggagaagtaagaggctacctcttacctaacatctgacctggcatctaattctgattctggctttaagccttcaaaactatagcttgcagaactgtagctgccatggctaggtagaagtgagcaaaaaagagttgggtgtctccttaagctgcagagatttctcattgacttttataaagcatgttcacccttatagtctaagactatatatataaatgtataaatatacagtatagatttttgggtggggggcattgagtattgtttaaaatgtaatttaaatgaaagaaaattgagttgcacttattgaccattttttaatttacttgttttggatggcttgtctatactccttcccttaaggggtatcatgtatggtgataggtatctagagcttaatgctacatgtgagtgcgatgatgtacagattctttcagttctttggattctaaatacatgccacatcaaacctttgagtagatccatttccattgcttattatgtaggtaagactgtagatatgtattcttttctcagtgttggtatattttatattactgacatttcttctagtgatgatggttcacgttggggtgatttaatccagttataagaagaagttcatgtccaaacggtcctctttagtttttggttgggaatgaggaaaattcttaaaaggcccatagcagccagttcaaaaacacccgacgtcatgtatttgagcatatcagtaacccccttaaatttaatacccagataccttatcttacaatgttgattgggaaaacatttgctgcccattacagaggtattaaaactaaatttcactactagattgactaactcaaatacacatttgctactgttgtaagaattc

SEQ ID NO: 88 - DNA, 1580 bp, human
catcctgccacccctagccttgctggggacgtgaaccctctccccgcgcctgggaagccttcttggcaccgggacccggagaatccccacggaagccagttccaaaagggatgaaaagggggcgtttcgggcactgggagaagcctgtattccagggcccctcccagagcaggaatctgggacccaggagtgccagcctcacccacgcagatcctggccatgagagctccgcacctccacctctccgccgcctctggcgcccgggctctggcgaagctgctgccgctgctgatggcgcaactctgggccgcagaggcggcgctgctcccccaaaacgacacgcgcttggaccccgaagcctatggctccccgtgcgcgcgcggctcgcagccctggcaggtctcgctcttcaacggcctctcgttccactgcgcgggtgtcctggtggaccagagttgggtgctgacggccgcgcactgcggaaacaagccactgtgggctcgagtaggggatgaccacctgctgcttcttcagggagagcagctccgccggaccactcgctctgttgtccatcccaagtaccaccagggctcaggccccatcctgccaaggcgaacggatgagcacgatctcatgttgctgaagctggccaggcccgtagtgctggggccccgcgtccgggccctgcagcttccctaccgctgtgctcagcccggagaccagtgccaggttgctggctggggcaccacggccgcccggagagtgaagtacaacaagggcctgacctgctccagcatcactatcctgagccctaaagagtgtgaggtcttctaccctggcgtggtcaccaacaacatgatatgtgctggactggaccggggccaggacccttgccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccctgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatggtgattgtcatgtaaggcttaacacagtgggtggtgagttctgactaaaggttacctgttgtcgtga

SEQ ID NO: 89 - DNA, 3359 bp, human
cacaccttcggcagcaggagggcggcagcttctcgcaggcggcagggcgggcggccaggatcatgtccaccaccacatgccaagtggtggcgttcctcctgtccatcctggggctggccggctgcatcgcggccaccgggatggacatgtggagcacccaggacctgtacgacaaccccgtcacctccgtgttccagtacgaagggctctggaggagctgcgtgaggcagagttcaggcttcaccgaatgcaggccctatttcaccatcctgggacttccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtgtaatgctctaagacctctcagcacgggcggaagaaactcccggagagctcacccaaaaaacaaggagatcccatctagatttcttcttgcttttgactcacagctggaagttagaaaagcctcgatttcatctttggagaggccaaatggtcttagcctcagtctctgtctctaaatattccaccataaaacagctgagttatttatgaattagaggctatagctcacattttcaatcctctatttctttttttaaatataactttctactctgatgagagaatgtggttttaatctctctctcacattttgatgatttagacagactccccctcttcctcctagtcaataaacccattgatgatctatttcccagcttatccccaagaaaacttttgaaaggaaagagtagacccaaagatgttattttctgctgtttgaattttgtctccccacccccaacttggctagtaataaacacttactgaagaagaagcaataagagaaagatatttgtaatctctccagcccatgatctcggttttcttacactgtgatcttaaaagttaccaaaccaaagtcattttcagtttgaggcaaccaaacctttctactgctgttgacatcttcttattacagcaacaccattctaggagtttcctgagctctccactggagtcctctttctgtcgcgggtcagaaattgtccctagatgaatgagaaaattattttttttaatttaagtcctaaatatagttaaaataaataatgttttagtaaaatgatacactatctctgtgaaatagcctcacccctacatgtggatagaaggaaatgaaaaaataattgctttgacattgtctatatggtactttgtaaagtcatgcttaagtacaaattccatgaaaagctcactgatcctaattctttccctttgaggtctctatggctctgattgtacatgatagtaagtgtaagccatgtaaaaagtaaataatgtctgggcacagtggctcacgcctgtaatcctagcactttgggaggctgaggaggaaggatcacttgagcccagaagttcgagactagcctgggcaacatggagaagccctgtctctacaaaatacagagagaaaaaatcagccagtcatggtggcctacacctgtagtcccagcattccgggaggctgaggtgggaggatcacttgagcccagggaggttggggctgcagtgagccatgatcacaccactgcactccagccaggtgacatagcgagatcctgtctaaaaaaataaaaaataaataatggaacacagcaagtcctaggaagtaggttaaaactaattctttaaaaaaaaaaaaaagttgagcctgaattaaatgtaatgtttccaagtgacaggtatccacatttgcatggttacaagccactgccagttagcagtagcactttcctggcactgtggtcggttttgttttgttttgctttgtttagagacggggtctcactttccaggctggcctcaaactcctgcactcaagcaattcttctaccctggcctcccaagtagctggaattacaggtgtgcgccatcacaactagctggtggtcagttttgttactctgagagctgttcacttctctgaattcacctagagtggttggaccatcagatgtttgggcaaaactgaaagctctttgcaaccacacaccttccctgagcttacatcactgcccttttgagcagaaagtctaaattccttccaagacagtagaattccatcccagtaccaaagccagataggccccctaggaaactgaggtaagagcagtctctaaaaactacccacagcagcattggtgcaggggaacttggccattaggttattatttgagaggaaagtcctcacatcaatagtacatatgaaagtgacctccaaggggattggtgaatactcataaggatcttcaggctgaacagactatgtctggggaaagaacggattatgccccattaaataacaagttgtgttcaagagtcagagcagtgagctcagaggcccttctcactgagacagcaacatttaaaccaaaccagaggaagtatttgtggaactcactgcctcagtttgggtaaaggatgagcagacaagtcaactaaagaaaaaagaaaagcaaggaggagggttgagcaatctagagcatggagtttgttaagtgctctctggatttgagttgaagagcatccatttgagttgaaggccacagggcacaatgagctctcccttctaccaccagaaagtccctggtcaggtctcaggtagtgcggtgtggctcagctgggtttttaattagcgcattctctatccaacatttaattgtttgaaagcctccatatagttagattgtgctttgtaattttgttgttgttgctctatcttattgtatatgcattgagtattaacctgaatgttttgttacttaaatattaaaaacactgttatcctacagtt

SEQ ID NO: 90 - DNA, 733 bp, human
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 91 - DNA, 1607 bp, human; identical to SEQ ID NO: 74

SEQ ID NO: 92 - DNA, 1753 bp, human; identical to SEQ ID NO: 75

SEQ ID NO: 93 - DNA, 2276 bp, human
aagcccagcagccccggggcggatggctccggccgcctggctccgcagcgcggccgcgcgcgccctcctgcccccgatgctgctgctgctgctccagccgccgccgctgctggcccgggctctgccgccggacgcccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttcttccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctgccaacactttcctctgaccatggcttggatgccctcaggggtgctgacccctgccaggccacgaatatcaggctagagacccatggccatctttgtggctgtgggcaccaggcatgggactgagcccatgtctcctcagggggatggggtggggtacaaccaccatgacaactgccgggagggccacgcaggtcgtggtcacctgccagcgactgtctcagactgggcagggaggctttggcatgacttaagaggaagggcagtcttgggcccgctatgcaggtcctggcaaacctggctgccctgtctccatccctgtccctcagggtagcaccatggcaggactgggggaactggagtgtccttgctgtatccctgttgtgaggttccttccaggggctggcactgaagcaagggtgctggggccccatggccttcagccctggctgagcaactgggctgtagggcagggccacttcctgaggtcaggtcttggtaggtgcctgcatctgtctgccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactgctatcctccaaagccattgtaaatgtgtgtacagtgtgtataaaccttcttcttctttttttttttttaaactgaggattgtcattaaacacagttgttttctaaaaaaaaaaaaaaaa

SEQ ID NO: 94 - DNA, 7381 bp, human
tacagccccaaggtcgctccctctggggccctttcttccccattcttcccagcagcccaaagctctggtgggacaggggcagcccctggggagggaggagaggacccaggaacccggctaggagggtggcccacccatttccagtgtgacctgttcccattcccccatgtctcctcccatccctcccgccactcagctcaggctgatgagaagcagagcaacgggtgtatcggtgttttctttcctggtggggtagtggggtggggctgaggagagaaaagggtgattagcgtggggccccgccctcttttgtcctcttcccaggttccctggccccttcggagaaacgcacttggttcgggccagccgcctgaggggacgggctcacgtctgctcctcacactgcagctgctgggccgtggagcttccccagggagccagggggacttttgccgcagccatgaagggggcacgctggaggagggtcccctgggtgtccctgagctgcctgtgtctctgcctccttccgcatgtggtcccaggaaccacagaggacacattaataactggaagtaaaactcctgccccagtcacctcaacaggctcaacaacagcgacactagagggacaatcaactgcagcttcttcaaggacctctaatcaggacatatcagcttcatctcagaaccaccagactaagagcacggagaccaccagcaaagctcaaaccgacaccctcacgcagatgatgacatcaactcttttttcttccccaagtgtacacaatgtgatggagactgttacgcaggagacagctcctccagatgaaatgaccacatcatttccctccagtgtcaccaacacactcatgatgacatcaaagactataacaatgacaacctccacagactccactcttggaaacacagaagagacatcaacagcaggaactgaaagttctaccccagtgacctcagcagtctcaataacagctggacaggaaggacaatcacgaacaacttcctggaggacctctatccaagacacatcagcttcttctcagaaccactggactcggagcacgcagaccaccagggaatctcaaaccagcaccctaacacacagaaccacttcaactccttctttctctccaagtgtacacaatgtgacagggactgtttctcagaagacatctccttcaggtgaaacagctacctcatccctctgtagtgtcacaaacacatccatgatgacatcagagaagataacagtgacaacctccacaggctccactcttggaaacccaggggagacatcatcagtacctgttactggaagtcttatgccagtcacctcagcagccttagtaacagttgatccagaaggacaatcaccagcaactttctcaaggacttctactcaggacacaacagctttttctaagaaccaccagactcagagcgtggagaccaccagagtatctcaaatcaacaccctcaacaccctcacaccggttacaacatcaactgttttatcctcaccaagtggattcaacccaagtggaacagtttctcaggagacattcccttctggtgaaacaaccatctcatccccttccagtgtcagcaatacattcctggtaacatcaaaggtgttcagaatgccaatctccagagactctactcttggaaacacagaggagacatcactatctgtaagtggaaccatttctgcaatcacttccaaagtttcaaccatatggtggtcagacactctgtcaacagcactctcccccagttctctacctccaaaaatatccacagctttccacacccagcagagtgaaggtgcagagaccacaggacggcctcatgagaggagctcattctctccaggtgtgtctcaagaaatatttactctacatgaaacaacaacatggccttcctcattctccagcaaaggccacacaacttggtcacaaacagaactgccctcaacatcaacaggtgctgccactaggcttgtcacaggaaatccatctacaagggcagctggcactattccaagggtcccctctaaggtctcagcaataggggaaccaggagagcccaccacatactcctcccacagcacaactctcccaaaaacaacaggggcaggcgcccagacacaatggacacaagaaacggggaccactggagaggctcttctcagcagcccaagctatagtgtgattcagatgataaaaacggccacatccccatcttcttcacctatgctggatagacacacatcacaacaaattacaacggcaccatcaacaaatcattcaacaatacattccacaagcacctctcctcaggaatcaccagctgtttcccaaaggggtcacactcgagccccgcagaccacacaagaatcacaaaccacgaggtccgtctcccccatgactgacaccaagacagtcaccaccccaggttcttccttcacagccagtgggcactcgccctcagaaattgttcctcaggacgcacccaccataagtgcagcaacaacctttgccccagctcccaccgggaatggtcacacaacccaggccccgaccacagcactgcaggcagcacccagcagccatgatgccaccctggggccctcaggaggcacgtcactttccaaaacaggtgcccttactctggccaactctgtagtgtcaacaccagggggcccagaaggacaatggacatcagcctctgccagcacctcacctgacacagcagcagccatgacccatacccaccaggctgagagcacagaggcctctggacaaacacagaccagcgaaccggcctcctcagggtcacgaaccacctcagcgggcacagctaccccttcctcatccggggcgagtggcacaacaccttcaggaagcgaaggaatatccacctcaggagagacgacaaggttttcatcaaacccctccagggacagtcacacaacccagtcaacaaccgaattgctgtccgcctcagccagtcatggtgccatcccagtaagcacaggaatggcgtcttcgatcgtccccggcacctttcatcccaccctctctgaggcctccactgcagggagaccgacaggacagtcaagcccaacttctcccagtgcctctcctcaggagacagccgccatttcccggatggcccagactcagaggacaggaaccagcagagggtctgacactatcagcctggcgtcccaggcaaccgacaccttctcaacagtcccacccacacctccatcgatcacatccagtgggcttacatctccacaaacccagacccacactctgtcaccttcagggtctggtaaaaccttcaccacggccctcatcagcaacgccacccctcttcctgtcaccagcacctcctcagcctccacaggtcacgccacccctcttgctgtcagcagtgctacctcagcttccacagtatcctcggactcccctctgaagatggaaacatcaggaatgacaacaccgtcactgaagacagacggtgggagacgcacagccacatcaccaccccccacaacctcccagaccatcatttccaccattcccagcactgccatgcacacccgctccacagctgcccccatccccatcctgcctgagagaggagtttccctcttcccctatggggcaggcgccggggacctggagttcgtcaggaggaccgtggacttcacctccccactcttcaagccggcgactggcttcccccttggctcctctctccgtgattccctctacttcacagacaatggccagatcatcttcccagagtcagactaccagattttctcctaccccaacccactcccaacaggcttcacaggccgggaccctgtggccctggtggctccgttctgggacgatgctgacttctccactggtcgggggaccacattttatcaggaatacgagacgttctatggtgaacacagcctgctagtccagcaggccgagtcttggattagaaagatgacaaacaacgggggctacaaggccaggtgggccctaaaggtcacgtgggtcaatgcccacgcctatcctgcccagtggaccctcgggagcaacacctaccaagccatcctctccacggacgggagcaggtcctatgccctgtttctctaccagagcggtgggatgcagtgggacgtggcccagcgctcaggcaacccggtgctcatgggcttctctagtggagatggctatttcgaaaacagcccactgatgtcccagccagtgtgggagaggtatcgccctgatagattcctgaattccaactcaggcctccaagggctgcagttctacaggctacaccgggaagaaaggcccaactaccgtctcgagtgcctgcagtggctgaagagccagcctcggtggcccagctggggctggaaccaggtctcctgcccttgttcctggcagcagggacgacgggacttacgattccaacccgtcagcataggtcgctggggcctcggcagtaggcagctgtgcagcttcacctcttggcgaggaggcgtgtgctgcagctacgggccctggggagagtttcgtgaaggctggcacgtgcagcgtccttggcagttggcccaggaactggagccacagagctggtgctgccgctggaatgacaagccctacctctgtgccctgtaccagcagaggcggccccacgtgggctgtgctacatacaggcccccacagcccgcctggatgttcggggacccccacatcaccaccttggatggtgtcagttacaccttcaatgggctgggggacttcctgctggtcggggcccaagacgggaactcctccttcctgcttcagggccgcaccgcccagactggctcagcccaggccaccaacttcatcgcctttgcggctcagtaccgctccagcagcctgggccccgtcacggtccaatggctccttgagcctcacgacgcaatccgtgtcctgctggataaccagactgtgacatttcagcctgaccatgaagacggcggaggccaggagacgttcaacgccaccggagtcctcctgagccgcaacggctctgaggtctcggccagcttcgacggctgggccaccgtctcggtgatcgcgctctccaacatcctccacgcctccgccagcctcccgcccgagtaccagaaccgcacggaggggctcctgggggtctggaataacaatccagaggacgacttcaggatgcccaatggctccaccattcccccagggagccctgaggagatgcttttccactttggaatgacctggcagatcaacgggacaggcctccttggcaagaggaatgaccagctgccttccaacttcacccctgttttctactcacaactgcaaaaaaacagctcctgggctgaacatttgatctccaactgtgacggagatagctcatgcatctatgacaccctggccctgcgcaacgcaagcatcggacttcacacgagggaagtcagtaaaaactacgagcaggcgaacgccaccctcaatcagtacccgccctccatcaatggtggtcgtgtgattgaagcctacaaggggcagaccacgctgattcagtacaccagcaatgctgaggatgccaacttcacgctcagagacagctgcaccgacttggagctctttgagaatgggacgttgctgtggacacccaagtcgctggagccattcactctggagattctagcaagaagtgccaagattggcttggcatctgcactccagcccaggactgtggtctgccattgcaatgcagagagccagtgtttgtacaatcagaccagcagggtgggcaactcctccctggaggtggctggctgcaagtgtgacgggggcaccttcggccgctactgcgagggctccgaggatgcctgtgaggagccgtgcttcccgagtgtccactgcgttcctgggaagggctgcgaggcctgccctccaaacctgactggggatgggcggcactgtgcggctctggggagctctttcctgtgtcagaaccagtcctgccctgtgaattactgctacaatcaaggccactgctacatctcccagactctgggctgtcagcccatgtgcacctgccccccagccttcactgacagccgctgcttcctggctgggaacaacttcagtccaactgtcaacctagaacttcccttaagagtcatccagctcttgctcagtgaagaggaaaatgcctccatggcagaggtcaacgcctcggtggcatacagactggggaccctggacatgcgggcctttctccgcaacagccaagtggaacgaatcgattctgcagcaccggcctcgggaagccccatccaacactggatggtcatctcggagttccagtaccgccctcggggcccggtcattgacttcctgaacaaccagctgctggccgcggtggtggaggcgttcttataccacgttccacggaggagtgaggagcccaggaacgacgtggtcttccagcccatctccggggaagacgtgcgcgatgtgacagccctgaacgtgagcacgctgaaggcttacttcagatgcgatggctacaagggctacgacctggtctacagcccccagagcggcttcacctgcgtgtccccgtgcagtaggggctactgtgaccatggaggccagtgccagcacctgcccagtgggccccgctgcagctgtgtgtccttctccatctacacggcctggggcgagcactgtgagcacctgagcatgaaactcgacgcgttcttcggcatcttctttggggccctgggcggcctcttgctgctgggggtcgggacgttcgtggtcctgcgcttctggggttgctccggggccaggttctcctatttcctgaactcagctgaggccttgccttgaaggggcagctgtggcctaggctacctcaagactcacctcatccttaccgcacatttaaggcgccattgcttttgggagactggaaaagggaaggtgactgaaggctgtcaggattcttcaaggagaatgaatactgggaatcaagacaagactataccttatccataggcgcaggtgcacagggggaggccataaagatcaaacatgcatggatgggtcctcacgcagacacacccacagaaggacactagcctgtgcacgcgcgcgtgcacacacacacacacacacacgagttcataatgtggtgatggccctaagttaagcaaaatgcttctgcacacaaaactctctggtttacttcaaattaactctatttaaataaagtctctctgactttttgtgtctccaaaa

SEQ ID NO: 95 - DNA, 2323 bp, human
agctatgatcgcaacaccttggtggccatcgtggtgggtgtggggcgcctcatcactggcatggaccgaggcctcatgggcatgtgtgtcaacgagcggcgacgcctcattgtgcctccccacctgggctatgggagcatcggcctggcggggctcattccaccggatgccaccctctacttcgatgtggttctgctggatgtgtggaacaaggaagacaccgtgcaggtgagcacattgctgcgcccgccccactgcccccgcatggtccaggacggcgactttgtccgctaccactacaatggcaccctgctggacggcacctccttcgacaccagctacagtaagggcggcacttatgacacctacgtcggctctggttggctgatcaagggcatggaccaggggctgctgggcatgtgtcctggagagagaaggaagattatcatccctccattcctggcctatggcgagaaaggctatgggacagtgatccccccacaggcctcgctggtctttcacgtcctcctgattgacgtgcacaacccgaaggacgctgtccagctagagacgctggagctcccccccggctgtgtccgcagagccggggccggggacttcatgcgctaccactacaatggctccttgatggacggcaccctcttcgattccagctactcccgcaaccacacctacaatacctatatcgggcagggttacatcatccccgggatggaccaggggctgcagggtgcctgcatgggggaacgccggagaattaccatccccccgcacctcgcctatggggagaatggaactggagacaagatccctggctctgccgtgctaatcttcaacgtccatgtcattgacttccacaaccctgcggatgtggtggaaatcaggacactgtcccggccatccgagacctgcaatgagaccaccaagcttggggactttgttcgataccattacaactgttctttgctggacggcacccagctgttcacctcgcatgactacggggccccccaggaggcgactctcggggccaacaaggtgatcgaaggcctggacacgggcctgcagggcatgtgtgtgggagagaggcggcagctcatcgtgcccccgcacctggcccacggggagagtggagcccggggagtcccaggcagtgctgtgctgctgtttgaggtggagctggtgtcccgggaggatgggctgcccacaggctacctgtttgtgtggcacaaggaccctcctgccaacctgtttgaagacatggacctcaacaaggatggcgaggtccctccggaggagttctccaccttcatcaaggctcaagtgagtgagggcaaaggacgcctcatgcctgggcaggaccctgagaaaaccataggagacatgttccagaaccaggaccgcaaccaggacggcaagatcacagtcgacgagctcaagctgaagtcagatgaggacgaggagcgggtccacgaggagctctgaggggcagggagcctggccaggcctgagacacagaggcccactgcgagggggacagtggcggtgggactgacctgctgacagtcaccctccctctgctgggatgaggtccaggagccaactaaaacaatggcagaggagacatctctggtgttcccaccaccctagatgaaaatccacagcacagacctctaccgtgtttctcttccatccctaaaccacttccttaaaatgtttggatttgcaaagccaatttggggcctgtggagcctggggttggatagggccatggctggtcccccaccatacctcccctccacatcactgacacagctgagcttgttatccatctccccaaactttctctttctttgtacttcttgtcatccccactcccagcccctattcctctatgtgacagctggctaggacccctctgccttccttcccaatcctgactggctcctagggaaggggaaggctcctggagggcagccctacctctcccatgccctttgccctcctccctcgcctccagtggaggctgagctgaccctgggctgctggaggccagactgggctgtagttagcttttcatccctaaagaaggctttccctaaggaaccatagaagagaggaagaaaacaaagggcatgtgtgagggaagctgcttgggtgggtgttagggctatgaaatcttggatttggggctgaggggtgggagggagggcagagctctgcacactcaaaggctaaactggtgtcagtccttttttcctttgttccaaataaaagattaaaccaaaaaaaaaaaaaaaaa

SEQ ID NO: 96 - DNA, 741 bp, human
tcacgtgacccgggcgcgctgcggccgcccgcgcggacccggcgagaggcggcggcgggagcggcggtgatggacgggtccggggagcagcccagaggcggggggcccaccagctctgagcagatcatgaagacaggggcccttttgcttcaggggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcgctcaccatctggaagaagatgggctgaggcccccagctgccttggactgtgtttttcctccataaattatggcatttttctgggaggggtggggattgggggacatgggcatttttcttacttttgtaattattggggggtgtggggaagagtggtcttgagggggtaataaacctccttcgggacacaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 97 - DNA, 2373 bp, human
cccaggcccaccccacccagcacccctggcgcagggactgctggaacctggctgtgcgcgctgtcgctttaagacagactctgccggcgccgtccggagccttagaaaccggccccggatcgcgagccggagccggagccggagccggggccggccgggctgctgaggcccgagcggcaggagcgcagcgcggagcgctgagccaggcgcccagtcgcgagaagctgccgccgcctctgcccgcccggcgccgcagccccgggcggtccatggggcgggcacggcgtcgctgcaggcgccggcagccctggagggcagccgcttaggcgctgcgctcttgtccccgcaggtcgcagccagggcggcggggcgcgcccagccccggcccctggagcgcccgccgcggtccccacctccatggacgccttcaaggggggcatgagcctggagcggctgccggaggggctccggccgccgccgccgccaccccatgacatggggcccgccttccacctggcccggcccgccgacccccgcgagccgctcgagaactccgccagcgagtcgtctgacacggagctgccagagaaggagcgcggcggggaacccaaggggcccgaggacagtggtgcgggaggcacgggctgcggcggcgcagacgacccagccaagaagaagaagcagcggcggcaacgtacgcacttcacaagccagcagttgcaagagctagaggccacgttccagaggaaccgctaccccgacatgagcatgagggaggagatcgccgtgtggaccaacctcaccgagccgcgcgtgcgggtctggttcaagaaccggcgagccaagtggcgtaagcgcgagcgtaaccagcagctggacctgtgcaagggtggctacgtgccgcagttcagcggcctagtgcagccctacgaggacgtgtacgccgccggctactcctacaacaactgggccgccaagagcctggcgccagcgccgctctccaccaagagcttcaccttcttcaactccatgagcccgctgtcgtcgcagtccatgttctcagcacccagctccatctcctccatgaccatgccgtccagcatgggcccaggcgccgtgcctggcatgcccaactcgggcctcaacaacatcaacaacctcaccggctcctcgctcaactcggccatgtcgccgggcgcttgcccgtacggcactcccgcctcgccctacagcgtctaccgggacacgtgcaactcgagcctagccagcctgcggctcaagtccaaacagcactcgtcgtttggctacggcgccctgcagggcccggcctcgggcctcaacgcgtgccagtacaacagctgaccgccccgccgcaccacgcgggccggcggccggagcggggaagggcgcgggcgcggaggacgcacgcggggccccggctcgcaagccccagctcaccgcgccgcggacctcacacctgcgcagccccctcctcccacttcccactccgggttggttttgtgtttgcttttccggaccccactctgccctccaaaaagacaaaaaaaaaaaaaaaaaaaaaagcaaaaagacgtcggagaaaagtgccgcgaaaaaatggatgagttgcaatttctctcgggatggcgcgggtggtgtgtgtgtgttcccacgggccccggaggcccactccgcggagggcacgcggcgcggtaggcgagcgccgaggcccagcggccgggggaggacgacctcgtatcccgcgtccccgccgcgctggatccggactgagtggccgggcctgcggactggatgtgcggggcctggacttgcctaggatttcccgaccccgtacaaaccaagttgccctctccgagctaggcccggccgagagcgccttagctcgagtcggatccgtgttggggcgggcgttgggtttggggggacggtgcccccagcccaggatcgggcactcagtggagccgcacacggccccggcgcgcctggtagagcctcgctggccccgcgccccggagccctatattaaggccacggagcgacagcgggcagtgcgggcctggcgggaggtgggggaggtccatctcagaacaccccagccttgagcttagctgcaggcccaggccctctgctctgctcccgggctaggaggtggccctctgtctgggcgaacagccccctcctcaccgcccgccgtgcaagagtcgagccggcagagcaaggggcgcggccccagggccctgcgcccactttgcacacccgctctccggcccgcgcccctgtttacagcgtccctgtgtatgttggactgactgtaataaatctgtctatatcgactaaaaaaaaaaaaaaaaa

SEQ ID NO: 98 - DNA, 1314 bp, human
aattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatccgagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactaaaaaaaaaaaaaaaaaaaaaaaaaaaaaaaaaaaaaaaaaaaaaaaaaaaataaaaaaaaaaaaaaaaaa